(19) United States
(12) Patent Application Publication
(10) Pub. No.: US 2021/0407623 A1
(43) Pub. Date: Dec. 30, 2021

(54) DETERMINING TUMOR FRACTION FOR A SAMPLE BASED ON METHYL BINDING DOMAIN CALIBRATION DATA

(71) Applicant: GUARDANT HEALTH, INC., Redwood City, CA (US)

(72) Inventors: Oscar WESTESSON, Berkeley, CA (US); Yupeng He, Redwood City, CA (US); William J. GREENLEAF, Redwood City, CA (US); Ariel JAIMOVICH, Redwood City, CA (US); AmirAli TALASAZ, Atherton, CA (US)

(21) Appl. No.: 17/219,338

(22) Filed: Mar. 31, 2021

Related U.S. Application Data
(60) Provisional application No. 63/002,824, filed on Mar. 31, 2020.

(51) Int. Cl. G16B 30/10; G16B 30/00; G16B 20/20

(52) U.S. Cl. CPC G16B 30/10; G16B 20/20; G16B 30/00

(57) ABSTRACT

The application is directed to systems and processes to determine an estimate for tumor fraction of a sample. In various examples, amounts of methylation of nucleic acids can be determined based on a strength of binding by the nucleic acids to methyl binding domain (MBD). The nucleic acids can be partitioned according to the strength of binding to MBD. Additionally, a number of cytosine-guanine regions for the nucleic acids can be determined. Amounts of methylation of classification regions of the nucleic acids can be determined based on the partition information associated with the nucleic acids and the number of cytosine-guanine regions of the nucleic acids. The classification regions can have differing amounts of methylation in tumor cells and non-tumor cells. The estimate for tumor fraction of the sample can be determined according to the amounts of methylation of the classification regions.

### CROSS-REFERENCE TO RELATED APPLICATION

This application claims the benefit of priority date of U.S. Provisional Patent Application No. 63/002,824, filed Mar. 31, 2020.

### SEQUENCE LISTING

This application includes sequences within txt file 2021-03-31_GH0061US_SEQLIST.txt of 2 kb created Mar. 31, 2021, which is incorporated by reference.

### BACKGROUND

Cancer is a major cause of disease worldwide. Each year, tens of millions of people are diagnosed with cancer around the world, and more than half eventually die from it. In many countries, cancer ranks the second most common cause of death following cardiovascular diseases. Early detection is associated with improved outcomes for many cancers.

Cancer can be caused by the accumulation of genetics variations within an individual's normal cells, at least some of which result in improperly regulated cell division. Such variations commonly include copy number variations (CNVs), single nucleotide variations (SNVs), gene fusions, insertions and/or deletions (indels), epigenetic variations include 5-methylation of cytosine (5-methylcytosine) and association of DNA with chromatin and transcription factors.

Cancers are often detected by biopsies of tumors followed by analysis of cells, markers or DNA extracted from cells. But more recently it has been proposed that cancers can also be detected from cell-free nucleic acids in body fluids, such as blood or urine. Such tests have the advantage that they are noninvasive and can be performed without identifying suspected cancer cells in biopsy. However, such tests are complicated by the fact that the amount of nucleic acids in body fluids is very low and what nucleic acid are present are heterogeneous in form (e.g., RNA and DNA, single-stranded and double-stranded, and various states of post-replication modification and association with proteins, such as histones).

Thus, there is a need for improved systems and methods for improved cancer detection using liquid biopsy assays. Therefore, it is an object of the disclosure to provide computer-implemented systems and methods that have improved capability to classify a sample as containing tumor-derived DNA with heightened sensitivity.

### SUMMARY

It is to be understood that both the following general description and the following detailed description are illustrative and explanatory only and are not restrictive. Methods, systems, and apparatuses for classifying a sample as tumor-derived or not tumor-derived are described herein.

In one or more implementations, systems and processes are described to determine an estimate for tumor fraction of a sample. In various examples, amounts of methylation of nucleic acids can be determined based on a strength of binding by the nucleic acids to methyl binding domain (MBD), or other modified nucleotide specific binding reagent. The nucleic acids can be partitioned according to the strength of binding to MBD. Additionally, a number of cytosine-guanine regions for the nucleic acids can be determined. Amounts of methylation of classification regions of the nucleic acids can be determined based on the partition information associated with the nucleic acids and the number of cytosine-guanine regions of the nucleic acids. The classification regions can have differing amounts of methylation in tumor cells and non-tumor cells. The estimate for tumor fraction of the sample can be determined according to the amounts of methylation of the classification regions.

In various implementations, methods are described comprising obtaining, by a computing system including one or more processors and memory, sequencing data including sequencing reads, individual sequencing reads indicating a nucleotide sequence of a nucleic acid included in a sample and indicating a methyl binding domain (MBD) partition of a plurality of MBD partitions, the MBD partition indicating an amount of methylation of cytosine-guanine (CG) regions of the nucleotide sequence, wherein individual CG regions of the nucleic acid sequence include at least a threshold number of cytosine-guanine pairs; analyzing, by the computing system, the sequencing data to determine a first subset of the sequencing reads that include one or more control regions, the one or more control regions having at least a threshold number of methylated CG regions; determining, by the computing system, a first number of sequencing reads of the first subset of sequencing reads that correspond to a first partition of the plurality of partitions, the first partition corresponding to a first range of numbers of methylated CG regions; determining, by the computing system, a second number of sequencing reads of the first subset of sequencing reads that correspond to a second partition of the plurality of partitions, the second partition corresponding to a second range of numbers of methylated CG regions that is different from the first range of numbers of methylated CG regions; generating, by the computing system, MBD binding calibration data that indicates a first number of probabilities of additional nucleic acids being associated with the first partition and a second number of probabilities of the additional nucleic acids included in the sample being associated with the second partition, wherein the MBD binding calibration data is generated based on the first number of sequencing reads of the subset of sequencing reads and the second number of sequencing reads of the subset of sequencing reads; analyzing, by the computing system, the sequencing data to determine a second subset of the sequencing reads that include one or more classification regions, the one or more classification regions including at least one of a first CG region that has at least a first threshold amount of methylation in cells derived from a tumor and a second CG region that has no greater a second threshold amount of methylation in non-tumor derived cells, the first threshold amount of methylation being greater than the second threshold amount of methylation; determining, by the computing system, partition data for the second subset of the sequencing reads based on partition tags included in the second subset of the sequencing reads, the partition data for individual sequencing reads of the second subset of sequencing reads indicating a partition of the plurality of partitions corresponding to the individual sequencing reads and individual partition tags including a nucleotide sequence corresponding to a partition of the plurality of partitions; determining, by the computing system, CG region data that indicates a number of CG regions of individual nucleic acids that correspond to the individual sequencing reads of the second subset of sequencing reads; determining, by the computing system, an amount of methylation of the number of CG regions in individual classification regions of the one or more classification regions based on an analysis of the partition data and the CG region data in relation to the MBD calibration data; and determining, by the computing system, an estimate for tumor fraction of the sample data by maximizing a likelihood of the estimate of the tumor fraction based on the amounts of methylation of the one or more classification regions, the estimate for tumor fraction indicating a number of nucleic acids included in the sample derived from a tumor.

Additional features and implementations will be set forth in part in the description which follows. The features and implementations will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein.

FIG. 1 shows flow diagram of an example process to classify a sample as cancer or non-cancer.

FIG. 2 shows flow diagram of an example method to partition nucleic acids according to MBD binding.

FIG. 3 shows a schematic diagram of example process to model determining tumor fraction based on MBD partitioning information.

FIG. 4 shows a diagram of an example method to identify partitions associated with nucleic acids based on molecular barcodes.

FIG. 5 shows a flow diagram of an example method to classify a sample as tumor-derived or not tumor-derived.

FIG. 6 shows an example method directed to molecule filtering based on amounts of methylation of molecules.

FIG. 7 shows example MBD binding curves.

FIG. 8 shows estimations of MBD binding curves.

FIG. 9 shows a schematic of a multimodal approach to colorectal cancer detection: cfDNA extraction, library prep, and partitioning workflow.

FIG. 10 shows an epigenomic estimation of tumor fraction correlates with genomic estimation.

FIG. 11 shows a cancer detection assay detecting differential methylation patterns in regions associated with differential methylation in CRC tumors.

FIG. 12 shows an example computing device to determine estimates for tumor fraction of a sample.

FIG. 13 shows a flow diagram of an example method to classify a sample as tumor or normal.

FIG. 14 illustrates a flow diagram of a process 1400 to determine an estimate for tumor fraction of a sample based on calibration data and partitioning of nucleic acids according to binding with methyl binding domain (MBD).

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

The term “subject” may refer to an animal, such as a mammalian species (preferably human) or avian (e.g., bird) species. More specifically, a subject can be a vertebrate, e.g., a mammal such as a mouse, a primate, a simian or a human. Animals include farm animals, sport animals, and pets. A subject can be a healthy individual, an individual that has symptoms or signs or is suspected of having a disease or a predisposition to the disease, or an individual that is in need of therapy or suspected of needing therapy. In some implementations, the subject is human, such as a human who has, or is suspected of having, cancer.

The phrase “cell-free nucleic acid” may refer to nucleic acids not contained within or otherwise bound to a cell, or in other words, nucleic acids remaining in a sample after removing intact cells. Cell-free nucleic acids can be referred to as non-encapsulated nucleic acid sourced from a bodily fluid (e.g., blood, urine, CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or partially double- and single-stranded. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA (ctDNA). Others are released from healthy cells. ctDNA can be non-encapsulated tumor-derived fragmented DNA. Cell-free fetal DNA (cffDNA) is fetal DNA circulating freely in the maternal blood stream. A cell-free nucleic acid can have one or more associated epigenetic modifications, for example, can be acetylated, 5-methylated, ubiquitylated, phosphorylated, sumoylated, ribosylated, and/or citrullinated. In some implementations, cell-free nucleic acid is cfDNA, which usually includes double-stranded cfDNA.

The phrase “nucleic acid tag” may refer to a short nucleic acid (e.g., less than 500, 100, 50, or 10 nucleotides long), used to label nucleic acid molecules to distinguish nucleic acids from different samples (e.g., representing a sample index), or different nucleic acid molecules in the same sample (e.g., representing a molecular barcode), or different nucleic acid molecules in the same partition (e.g. representing a partition tag), of different types, or which have undergone different processing. Tags can be single stranded, double-stranded or at least partially double-stranded. Tags can have the same length or varied lengths. Tags can be blunt-end or have an overhang. Tags can be attached to one end or both ends of the nucleic acids. Nucleic acid tags can be decoded to reveal information such as the sample of origin, form or processing of a nucleic acid. Tags can be used to allow pooling and parallel processing of multiple samples comprising nucleic acids bearing different molecular barcodes and/or sample indexes with the nucleic acids subsequently being deconvolved by reading the molecular barcodes. Additionally or alternatively, nucleic acid tags can be used to distinguish different molecules in the same sample (i.e., molecular barcode). This includes both uniquely tagging different molecules in the sample, or non-uniquely tagging the molecules in the sample. In the case of non-unique tagging, a limited number of different molecular barcodes may be used to tag molecules such that different molecules can be distinguished based on their start and/or stop position where they map on a reference genome (i.e., genomic coordinates) in combination with at least one molecular barcode. A sufficient number of different molecular barcodes are used such that there is a low probability (e.g. <10%, <5%, <1%, or <0.1%) that any two molecules having the same start/stop also have the same molecular barcode. Some tags include multiple identifiers to label samples, forms of molecule within a sample, and molecules within a form having the same start and stop points. Such tags can exist in the form Ali, wherein the letter indicates a sample type, the Arabic number indicates a form of molecule within a sample, and the Roman numeral indicates a molecule within a form.

The term “adapter” refers to a short nucleic acid (e.g., less than 500, 100, or 50 nucleotides long) usually at least partly double-stranded for linkage to either or both ends of a sample nucleic acid molecule. Adapters can include primer binding sites to permit amplification of a nucleic acid molecule flanked by adapters at both ends, and/or a sequencing primer binding site, including primer binding sites for next generation sequencing (NGS). Adapters can also include binding sites for capture probes, such as an oligonucleotide attached to a flow cell support. Adapters can also include a tag (e.g. molecular barcode) as described above. Tags are preferably positioned relative to primer and sequencing primer binding sites, such that a tag is included in amplicons and sequencing reads of a nucleic acid molecule. Adapters of the same or different sequences can be linked to the respective ends of a nucleic acid molecule. Sometimes adapters of the same sequence are linked to the respective ends except that the molecular barcode is different. In one or more examples, an adapter can be a Y-shaped adapter in which one end is blunt ended or tailed, for joining to a nucleic acid molecule, which is also blunt ended or tailed with one or more complementary nucleotides. In one or more additional examples, an adapter can be a bell-shaped adapter, likewise with a blunt or tailed end for joining to a nucleic acid to be analyzed.

As used herein, the terms “sequencing” or “sequencer” refer to any of a number of technologies used to determine the sequence of a biomolecule, e.g., a nucleic acid such as DNA or RNA. Example sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In one or more implementations, sequencing can be performed by a gene analyzer such as, for example, gene analyzers commercially available from Illumina or Applied Biosystems.

The phrase “next generation sequencing” or NGS refers to sequencing technologies having increased throughput as compared to traditional Sanger- and capillary electrophoresis-based approaches, for example, with the ability to generate hundreds of thousands of relatively small sequence reads at a time. Some examples of next generation sequencing techniques include, but are not limited to, sequencing by synthesis, sequencing by ligation, and sequencing by hybridization.

The term “DNA (deoxyribonucleic acid)” refers to a chain of nucleotides comprising deoxyribonucleosides that each comprise one of four nucleobases, namely, adenine (A), thymine (T), cytosine (C), and guanine (G). The term “RNA (ribonucleic acid)” refers to a chain of nucleotides comprising four types of ribonucleosides that each comprise one of four nucleobases, namely; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “nucleotide sequence”, “genomic sequence,” “genetic sequence,” or “fragment sequence,” or “nucleic acid sequencing read” denotes any information or data that is indicative of the order of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

A “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. A polynucleotide can comprise at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that “A” denotes adenosine, “C” denotes cytosine, “G” denotes guanosine, and “T” denotes thymidine, unless otherwise noted. The letters A, C, G, and T may be used to refer to the bases themselves, to nucleosides, or to nucleotides comprising the bases, as is standard in the art.

The phrase “reference sequence” refers to a known sequence used for purposes of comparison with experimentally determined sequences. For example, a known sequence can be an entire genome, a chromosome, or any segment thereof. A reference typically includes at least 20, 50, 100, 200, 250, 300, 350, 400, 450, 500, 1000, or more nucleotides. A reference sequence can align with a single contiguous sequence of a genome or chromosome or can include non-contiguous segments aligning with different regions of a genome or chromosome. In one or more implementations, the reference sequence is a human genome. Reference human genomes can include, e.g., hG19 and hG38.

The phrase “modified nucleotide specific binding reagent” as used herein, refers to a binding reagent that is specific for, or targets, modified nucleotides. For example, a modified nucleotide can be a nucleotide that has been methylated, thus, the binding reagent can be specific for a methylated nucleotide. Examples of binding reagents include, but are not limited to, a methyl binding domain (MBD) of a methylation binding protein (“MBP”) or variants thereof, an antibody (and antibody variants e.g., single chain antibodies), aptamers, or combinations thereof. Thus, as disclosed throughout, the use of MBD can be exchanged for any other modified nucleotide specific binding reagent, provided the modified nucleotide specific binding reagent has the desired specificity and affinity for the specific modified base of interest in the selected implementation.

The phrase “biological sample” as used herein, generally refers to a tissue or fluid sample derived from a subject. A biological sample may be directly obtained from the subject. The biological sample may be or may include one or more nucleic acid molecules, such as deoxyribonucleic acid (DNA) or ribonucleic acid (RNA) molecules. The biological sample can be derived from any organ, tissue, or biological fluid. A biological sample can comprise, for example, a bodily fluid or a solid tissue sample. An example of a solid tissue sample is a tumor sample, e.g., from a solid tumor biopsy. Bodily fluids include, for example, blood, serum, plasma, tumor cells, saliva, urine, lymphatic fluid, prostatic fluid, seminal fluid, milk, sputum, stool, tears, and derivatives of these. In one or more implementations, the biological sample is, or is derived from, blood.

The term “sequence reads” refers to nucleotide sequences read from a sample obtained from an individual as part of a sequencing process describe previously

The phrase “about” or “approximately” as applied to one or more values or elements of interest, refers to a value or element that is similar to a stated reference value or element. In certain implementations, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a non-transitory computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Existing methods to measure DNA methylation, such as sodium bisulfite conversion sequencing, often result in the loss of about 50%-80% of DNA included in a sample. In these instances, the loss of DNA can reduce the presence of one or more types of DNA such that the presence of the one or more types of DNA such as cfDNA, is difficult to detect. In one or more additional scenarios, existing methods to measure DNA methylation, such as enrichment or depletion methods, can have a relatively high level of resolution, such as about 100 base pairs (bp) to about 200 bp that can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determinations of tumor fraction estimates can impact diagnosis and/or treatment decisions for individuals.

The methods and systems described herein are directed to accurately generating information indicating the amounts of methylation of nucleic acids using data that indicates an amount of binding of nucleic acids to methyl binding domain (MBD). In various examples, the application is directed to systems and processes to determine an estimate for tumor fraction of a sample. In one or more examples, amounts of methylation of nucleic acids can be determined based on a strength of binding by the nucleic acids to methyl binding domain (MBD). The nucleic acids can be partitioned according to the strength of binding to MBD. Additionally, a number of cytosine-guanine (CG) regions for the nucleic acids can be determined. Amounts of methylation of classification regions of the nucleic acids can be determined based on the partition information associated with the nucleic acids and the number of cytosine-guanine regions of the nucleic acids. The classification regions can have differing amounts of methylation in tumor cells and non-tumor cells, e.g., the non-tumor cells can be blood cells giving rise to the non-cancer cell derived portion of a cfDNA sample. The estimate for tumor fraction of the sample can be determined according to the amounts of methylation of the classification regions.

By using the techniques and systems described herein, the accuracy of detecting the methylation rates of nucleic acids is increased with respect to existing techniques because, in contrast to existing techniques, such as sodium bisulfite conversion, relatively low amounts of nucleic acids are lost in generated the MBD binding data. In addition, the computational methods and systems described herein can determine the amounts of methylation of CG regions at a resolution that is improved with respect to enrichment or depletion techniques.

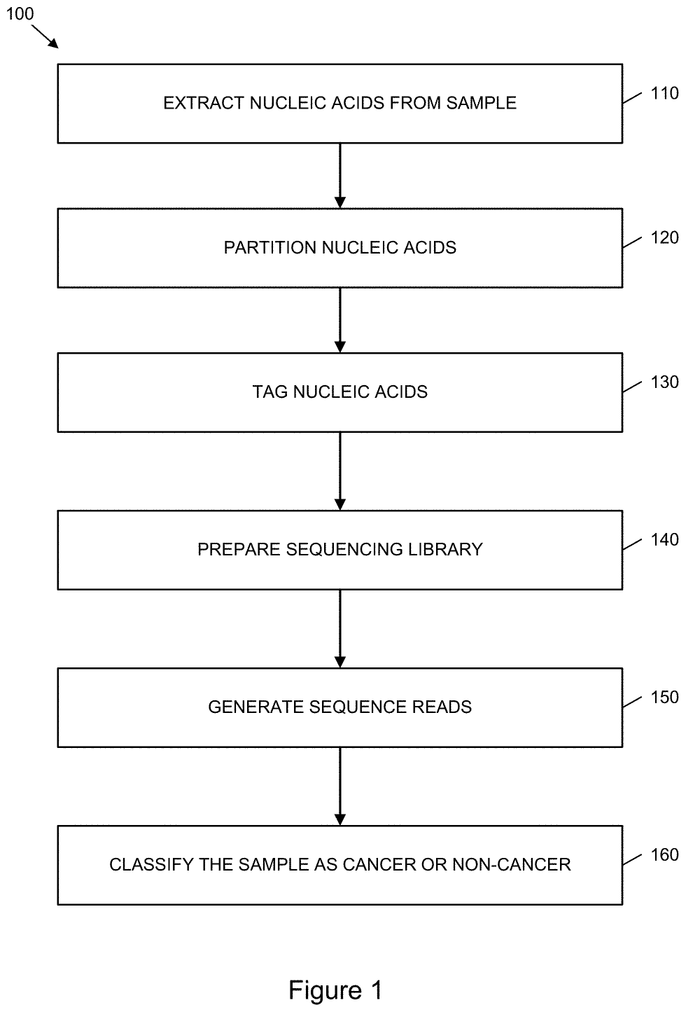

FIG. 1 is an example method 100 for detecting the presence of tumor-derived DNA in a sample. At step 110, nucleic acids (DNA or RNA) may be extracted from the sample. In one or more implementations, the nucleic acids comprise cell-free nucleic acids. In various implementations, the test sample may be a sample selected from one or more of blood, plasma, serum, urine, fecal, saliva samples, combinations thereof, and/or the like. In one or more additional examples, the biological sample may comprise a sample selected from one or more of whole blood, a blood fraction, a tissue biopsy, pleural fluid, pericardial fluid, cerebrospinal fluid, and peritoneal fluid. In one or more implementations, the test sample may comprise cell-free nucleic acids (e.g., cell-free DNA). For example, the test sample may be a cell-free nucleic acid sample taken from a subject's blood. In one or more implementations, the cell free nucleic acid sample may be extracted from a test sample obtained from a subject known to have cancer (e.g., a cancer patient), or a subject suspected of having cancer.

Samples can include nucleic acids varying in modifications including post-replication modifications to nucleotides and binding, usually noncovalently, to one or more proteins.

In one or more implementations, the population of nucleic acids is one obtained from a serum, plasma, or blood sample from a subject suspected of having cancer or previously diagnosed with cancer. The nucleic acids include ones having varying levels of methylation. Methylation can occur from any one or more post-replication or transcriptional modifications. Post-replication modifications include modifications of the nucleotide cytosine, including, but not limited to, 5-methylcytosine, 5-hydroxymethylcytosine, 5-formylcytosine and 5-carboxylcytosine.

The following description may be applicable to both DNA and RNA types of nucleic acid sequences. In various implementations, nucleic acids are extracted from the sample through a purification process. In one or more examples, the purification process of nucleic acids can include isolation by pelleting and/or precipitating the nucleic acids in a tube. In one or more implementations, nucleic acids can be further processed. For example, the cell free nucleic acid extracted from the test sample can be RNA that is then converted to DNA using reverse transcriptase.

In various aspects, the sample can include control nucleic acid sequences. Control nucleic acid sequences can also be referred to as “controls” or “spike-ins.” Control nucleic acid sequences can be exogenous sequences, meaning they are derived from somewhere other than the sample nucleic acid sequences. For example, the control can be a region of the lambda phage genome or human genome. In one or more instances, the control can be synthetic oligonucleotides. In one or more examples, the control can have one or more non-naturally occurring nucleotides. In one or more implementations, the control can have a combination of lambda phage genome, human genome, and non-naturally occurring sequences. The control has known numbers of methylated CG sites. Controls can be used to monitor an assay's ability to partition DNA with differential amounts of methylation into the respective bins.

The method 100 may comprise partitioning the nucleic acids at step 120. In one or more implementations, nucleic acids can be partitioned based on one or more characteristics of the nucleic acids. In one or more instances, nucleic acid sequences extracted from the sample at step 110 are partitioned into two or more partitions (e.g., at least 3, 4, 5, 6, 7, 8, 9, 10, 12, 14, 16, 18, 20 or more partitions). In various implementations, each partition may be differentially tagged at step 130, described below. Tagged partitions can then be pooled together for collective sample prep and/or sequencing at steps 140-150, described below. The partitioning-tagging-pooling steps 120-140 can occur more than once, with each round of partitioning occurring based on a different characteristics (examples provided herein), and tagged using differential tags that are distinguished from other partitions and partitioning means.

Examples of characteristics that can be used for partitioning include multiple different nucleotide modifications, methylation level, nucleosome binding, sequence mismatch, immunoprecipitation, and/or proteins that bind to DNA. Resulting partitions can include one or more of the following nucleic acid forms: double-stranded DNA (dsDNA), shorter DNA fragments and longer DNA fragments. Alternatively or additionally, a heterogeneous population of nucleic acids can be partitioned into nucleic acids with one or more epigenetic modifications and without the one or more epigenetic modifications. Examples of epigenetic modifications include, but are not limited to, presence or absence of methylation; level of methylation, hydroxymethylation, and type of methylation (5′ cytosine or 6 methyladenine).

Partitioning of the nucleic acids can be performed by contacting the nucleic acids with a modified nucleotide specific binding reagent, such as a MBD of a MBP. A modified nucleotide specific binding reagent can bind to 5-methylcytosine (5mC). The modified nucleotide specific binding reagent, such as a MBD, can be coupled to paramagnetic beads, such as Dynabeads® M-280 Streptavidin via a biotin linker. Partitioning into fractions with different extents of methylation can be performed by increasing the NaCl concentration in a series of washes. The sequences eluted from the modified nucleotide specific binding reagent are partitioned into two or more fractions (e.g., hypo, hyper) depending on which wash (e.g., NaCl concentration) eluted the sequences.

The binding of the nucleic acids with the modified nucleotide specific binding reagent can be a function of number of methylated (or modified) sites per molecule, with molecules having more methylation eluting under increased salt concentrations. To elute the DNA into distinct populations based on the extent of methylation, one can use a series of elution buffers of increasing NaCl concentration. Salt concentrations can, in one or more implementations, range from about 100 nm to about 2500 mM NaCl. In various implementations, the process results in three (3) partitions. Molecules are contacted with a solution at a first salt concentration and comprising a molecule comprising a methyl binding domain, which molecule can be attached to a capture moiety, such as streptavidin. At the first salt concentration a population of molecules will bind to the MBD and a population will remain unbound. The unbound population can be separated as a “hypomethylated” population (hypo partition). For example, a first partition representative of the hypomethylated form of DNA is that which remains unbound at a low salt concentration. In one or more illustrative examples, the concentration of NaCl of the solution used to produce the first partition can be about 100 nM, about 120 nM, about 140 nM, about 160 nM, about 180 nM, about 200 nM. or about 250 nM. A second partition (residual) representative of intermediate methylated DNA is eluted using an intermediate salt concentration, e.g., between 100 mM and 2000 mM concentration. In one or more additional illustrative examples, the concentration of NaCl of the solution used to produce the second partition can be from about 100 mM to about 500 mM, from about 100 mM to about 1000 mM, from about 100 mM to about 1500 mM, from about 250 mM to about 1000 mM, from about 250 mM to about 1500 mM, from about 500 mM to about 1500 mM, from about 250 mM to about 2000 mM, from about 500 mM to about 2000 mM, or from about 1000 mM to about 2000 mM. This is also separated from the sample. A third partition representative of hypermethylated form of DNA (hyper partition) is eluted using a high salt concentration, e.g., at least about 2000 mM. In one or more further illustrative examples, the concentration of NaCl of the solution used to produce the third partition can be from about 2000 mM to about 5000 mM, from about 2000 mM to about 4000 mM, from about 2000 mM to about 3500 mM, from about 2000 mM to about 3000 mM, or from about 2500 mM to about 4000 mM.

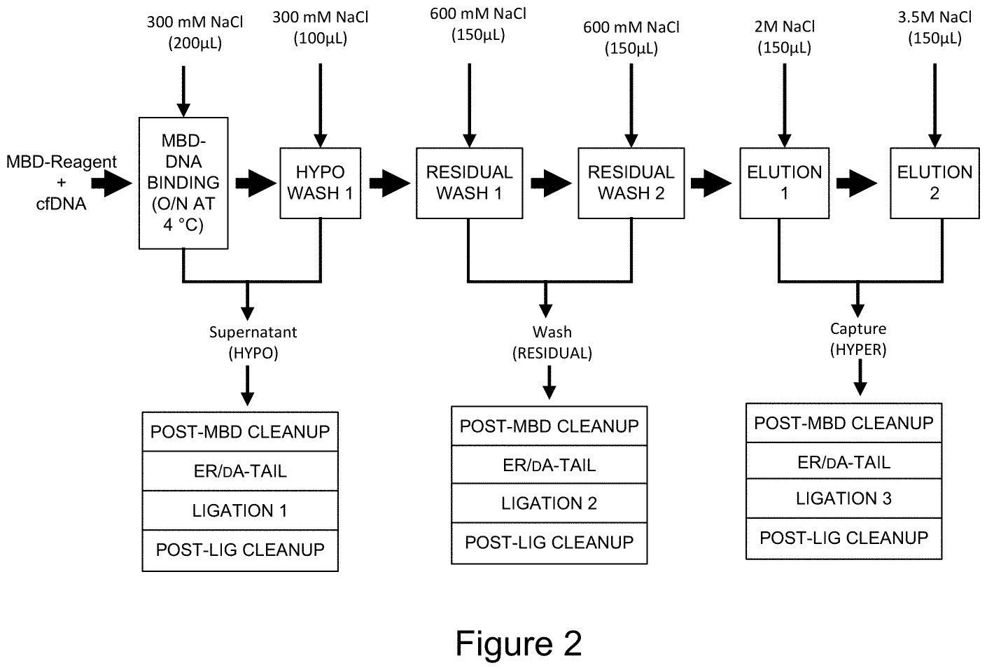

FIG. 2 is an example of the partitioning step 120. After allowing the nucleic acid sequences to bind to MBD, increasing salt concentrations can be used to wash the unbound or elute the bound nucleic acid sequences from the MBD into either Hypo, Residual, or Hyper partition bins. A 300 mM NaCl solution is used to wash the unbound or elute the weakly bound nucleic acid sequences into a Hypo partition. A 600 mM NaCl solution is used to elute the intermediately bound nucleic acid sequences into a Residual partition. The final washes use 2 M and 3.5 M NaCl solutions to elute the tightly bound nucleic acid sequences into a Hyper partition.

In one or more instances, controls can be partitioned in the same or similar way the nucleic acids from step 110 are partitioned. The controls can be added to the nucleic acids of step 110 so that the partitioning occurs to all the nucleic acids concurrently. Thus, the nucleic acids of step 110 are spiked with the controls. In various instances, the controls can be partitioned separate from the nucleic acids of step 110.

Returning to FIG. 1, the method 100 may comprise tagging the nucleic acids at step 130. The method 100 may optionally comprise tagging the nucleic acids prior to partitioning the nucleic acids at step 120. The tagging at step 130 can be independent of, or in addition to, any tagging that occurred prior to step 120. For example, the tagging prior to step 120 may be used to identify different nucleic acid forms or individual nucleic acid sequences and the tagging at step 130 may be used identify the partition to which nucleic acids belong.

In one or more implementations, each nucleic acid sequence in a sample can be differentially tagged. In various implementations, each form of a nucleic acid (e.g., ssDNA, dsDNA, RNA) can be differentially tagged. In one or more implementations, each partition can be differentially tagged. The tagging of each individual nucleic acid sequence present in the sample and/or the different forms of nucleic acids in the sample can occur prior to step 120 or after step 120. For example, all tagging can occur at step 130.

The controls can be tagged in a similar manner to the nucleic acid sequences in the sample (e.g., the nucleic acid sequences from step 110). Each control comprising a specific number of methylated CG molecules can be tagged with a particular barcode. For example, if six controls are used having 0, 1, 3, 5, 7, or 9 methylated CG molecules then six different barcodes can be used so that each control has its own barcode. This allows for easy identification of each control having a different number of CG molecules. The controls can further be tagged with a unique molecule identifier (UMI).

Tags can be molecules, such as nucleic acids, containing information that indicates a feature of the molecule with which the tag is associated. For example, molecules can bear a sample tag (which distinguishes molecules in one sample from those in a different sample), a partition tag (which distinguishes molecules in one partition from those in a different partition) or a molecular tag (which distinguishes different molecules from one another (in both unique and non-unique tagging scenarios). In various implementations, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a sample index, partition index or molecular index can be comprised of one barcode or a combination of two barcodes, each attached to different ends of a molecule. In one or more implementations, a molecular barcode is used to differentiate between molecules in the sample. In one or more implementations, a sample index is used to differentiate between different samples.

Tags can be used to label the individual nucleic acid sequences in a partition so as to correlate the tag (or tags) with a specific partition. In various implementations, a single tag can be used to label a specific partition. In one or more implementations, multiple different tags can be used to label a specific partition. In implementations employing multiple different tags to label a specific partition, the set of tags used to label one partition can be readily differentiated for the set of tags used to label other partitions. In one or more implementations, the tags may have additional functions, for example the tags can be used to index sample sources or used as unique molecular identifiers (which can be used to improve the quality of sequencing data by differentiating sequencing errors from mutations). Similarly, in one or more implementations, the tags may have additional functions, for example the tags can be used to index sample sources or used as non-unique molecular identifiers (which can be used to improve the quality of sequencing data by differentiating sequencing errors from mutations).

In various implementation, partition tagging comprises tagging molecules in each partition with the equivalent of a sample tag. After recombining partitions and sequencing molecules, the sample tags identify the source partition. In one or more additional implementation, different partitions are tagged with different sets of molecular tags, e.g., comprised of a pair of barcodes. In this way, each molecular barcode indicates the source partition as being useful to distinguish molecules within a partition. For example, a first set of 35 barcodes can be used to tag molecules in a first partition, while a second set of 35 barcodes can be used tag molecules in a second partition.

For example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition.

Identification of the different forms of nucleic acid in a sample can be achieved by differential tagging of the different forms of nucleic acid in the sample before the forms have been altered in a way that obfuscates their original form, such as by second-strand synthesis or amplification. Thus, in a nucleic acid including multiple forms, at least one form is linked to a nucleic acid tag to distinguish it from one or more other forms present in the sample. In a sample containing three forms of nucleic acid, such as single-stranded DNA, single-stranded RNA and double-stranded DNA, the three forms can be distinguished by differentially labelling at least two of the forms or by differentially labelling all three. The tags linked to nucleic acid molecules of the same form can be the same or different from one another. But if different from one another, the tags may, in one or more implementations, have part of their code in common so as to identify the molecules to which they are attached as being of a particular form. For example, nucleic molecules of a particular form can bear codes of the form A1, A2, A3, A4 and so forth, and those of a different form B1, B2, B3, B4 and so forth. Such a coding system allows distinction both between the forms and molecules within a form.

The method 100 may comprise preparation of a sequencing library at step 140. In one or more implementations, the tagging step of 130 can also be considered part of the preparation of a sequencing library at step 140. During library preparation, adapters, for example, include one or more sequencing oligonucleotides for use in subsequent cluster generation and/or sequencing (e.g., known P5 and P7 sequences for used in sequencing by synthesis (SBS) (Illumina, San Diego, Calif.)) may be ligated to the ends of the nucleic acid molecules through adapter ligation. In various implementations, unique molecular identifiers (UMI) may be added to the extracted nucleic acids during adapter ligation. The UMIs are short nucleic acid sequences (e.g., 4-10 base pairs) that are added to ends of nucleic acids during adapter ligation. In one or more implementations, UMIs are degenerate base pairs that serve as a unique tag that can be used to identify sequence reads obtained from nucleic acids. The UMIs can be further replicated along with the attached nucleic acids during amplification, which provides a way to identify sequence reads that originate from the same original nucleic acid segment in downstream analysis. The UMIs can be added to the controls as well.

The method 100 may comprise sequencing the nucleic acids in the sequencing library to generate sequence reads at 150. In one or more instances, each partition is differentially labelled, and the partitions are pooled together prior to sequencing. In one or more additional instances, the different partitions are separately sequenced. Sequence reads may be acquired by known means in the art. For example, a number of techniques and platforms obtain sequence reads directly from millions of individual nucleic acid (e.g., DNA such as cfDNA or gDNA or RNA such as cfRNA) molecules in parallel. Such techniques can be suitable for performing any of targeted gene panel sequencing, whole exome sequencing, whole genome sequencing, targeted gene panel bisulfite sequencing, and whole genome bisulfite sequencing.

Sample nucleic acids, optionally flanked by adapters, with or without prior amplification are generally subjected to sequencing. Sequencing methods or commercially available formats that are optionally utilized include, for example, Sanger sequencing, high-throughput sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may include multiple lanes, multiple channels, multiple wells, or other means of processing multiple sample sets substantially simultaneously. Sample processing units can also include multiple sample chambers to enable the processing of multiple runs simultaneously.

The sequencing reactions can be performed on one or more nucleic acid fragment types or regions known to contain markers of cancer or of other diseases. The sequencing reactions can also be performed on any nucleic acid fragment present in the sample. The sequence reactions may be performed on at least about 5%, 10%, 15%, 20%, 25%, 30%, 40%, 50%, 60%, 70%, 80%, 90%, 95%, 99%, 99.9%, or 100% of the genome. In one or more additional cases, sequence reactions may be performed on less than about 5%, 10%, 15%, 20%, 25%, 30%, 40%, 50%, 60%, 70%, 80%, 90%, 95%, 99%, 99.9%, or 100% of the genome.

Simultaneous sequencing reactions may be performed using multiplex sequencing techniques. In one or more implementations, cell-free polynucleotides are sequenced with at least about 1000, 2000, 3000, 4000, 5000, 6000, 7000, 8000, 9000, 10000, 50000, or 100,000 sequencing reactions. In one or more additional implementations, cell-free polynucleotides are sequenced with less than about 1000, 2000, 3000, 4000, 5000, 6000, 7000, 8000, 9000, 10000, 50000, or 100,000 sequencing reactions. Sequencing reactions are typically performed sequentially or simultaneously. Subsequent data analysis is generally performed on all or part of the sequencing reactions. In one or more implementations, data analysis is performed on at least about 1000, 2000, 3000, 4000, 5000, 6000, 7000, 8000, 9000, 10000, 50000, or 100,000 sequencing reactions. In one or more additional implementations, data analysis may be performed on less than about 1000, 2000, 3000, 4000, 5000, 6000, 7000, 8000, 9000, 10000, 50000, or 100,000 sequencing reactions. An example of a read depth is from about 1000 to about 50000 reads per locus (e.g., base position).

Sequencing at step 150 generates a plurality of sequence reads. The sequence reads may include sequences of nucleotide data less than about 150 bases in length, or less than about 90 bases in length. In certain implementations, sequence reads are between about 80 and about 90 bases, e.g., about 85 bases in length. In one or more implementations, methods described herein are applied to reads having lengths having less than about 50 or less than about 30 bases in length. Sequence read data can include the sequence data as well as meta information. Sequence read data can be stored in one or more file formats including, for example, VCF files, FASTA files or FASTQ files, as are known to those of skill in the art. FASTA and FASTQ are file formats used to store raw sequence reads from high throughput sequencing. FASTQ files store an identifier for each sequence read, the sequence, and the quality score string of each read. FASTA files store the identifier and sequence only. Other file formats are contemplated.

The method 100 of the modified base caller may comprise processing the sequence reads using a computational analysis to classify a sample as containing tumor-derived DNA or not containing tumor-derived DNA at step 160. Processing the sequence reads may comprise processing the sequence read data generated by sequencing at step 150. Generally, the method 100 of the modified base caller relies on the fact that circulating tumor DNA (ctDNA) in plasma exhibit DNA methylation patterns and/or rates distinct from that from somatic cells. A modified nucleotide binding reagent, such as Methyl Binding Domain (MBD), may be used to capture such epigenomic patterns. After extraction of DNA from plasma at step 110, the DNA may be partitioned into three bins at step 120: hyper methylated, residual (medium level methylation), and hypo methylated based on DNA affinity to a methylated-DNA binding protein. The DNA in each bin may then tagged with a molecular barcode (e.g., a distinct set of 35 by 35 unique molecular barcodes) at step 130 and then the DNA may be pooled back together for sequencing at step 140, and sequenced at step 150. At step 160, the methylation bin per molecule is detected in silico, based on the molecular barcode attached to each molecule. The modified base caller is configured to analyze the data generated by such partitioning, specifically the differential methylation of DNA fragments based on the partition of molecules to bins in order to detect circulating tumor DNA presence.

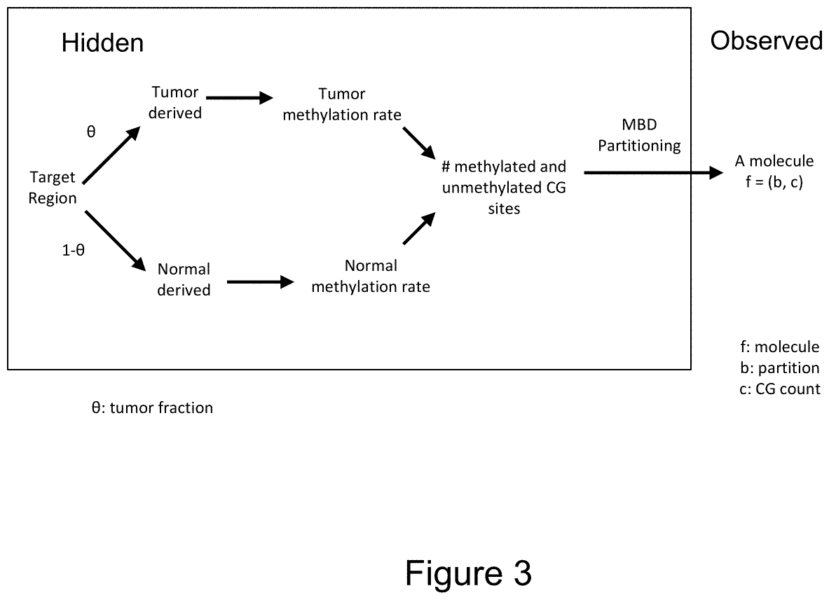

As shown in FIG. 3, the modified base caller may be configured to utilize the methylation data acquired to model the process that leads to an observed MBD partitioned molecule. Assuming the cfDNA of a sample is a mixture of tumor derived molecules and normal derived molecules, a molecule can either come from tumor cell or normal (blood) cell. Accordingly, there are two possible paths for a molecule to be observed:

a. The molecule is tumor derived.→Its methylation rate follows the distribution of tumor methylation rate.→The number of methylated and unmethylated CG sites follows binomial distribution determined by its methylation rate.→The molecule is partitioned by MBD.
b. The molecule is normal derived.→Its methylation rate follows the distribution of normal methylation rate.→The number of methylated and unmethylated CG sites follows binomial distribution determined by its methylation rate.→The molecule is partitioned by MBD.

Each step of the two paths in FIG. 3 has some uncertainty which can be measured by probability. The probability that a molecule is from a given path depends on the tumor fraction of the given region. Tumor fraction is the fraction of tumor derived molecules out of all molecules from a given region. If the tumor fraction is large, it is more likely that the observed molecule is tumor derived. In one or more implementations, the modified base caller is applied to genomic classification regions. In various implementations, the classification regions may be highly methylated in tumor tissue but lowly methylated in blood cells. Therefore, if a molecule is tumor derived, its methylation rate is likely to be high, whereas the methylation rate tends to be low for normal derived molecules. In one or more additional implementations, the classification regions may be lowly methylated in tumor tissue but highly methylated in blood cells. In various implementations, one more classification regions may be lowly methylated in tumor tissue but highly methylated in blood cells while one or more other classification regions may be highly methylated in tumor tissue but lowly methylated in blood cells. Given a methylation rate and the number of CG sites in the molecule, the number of methylated CG sites (and unmethylated CG sites) follow a binomial distribution. Given a certain methylated and unmethylated CG count, MBD partitions the molecule into hyper/residual/hypo partition with some probability.

The modified base caller may determine a likelihood (e.g., independent or joint) of observing one or more molecules from a given region or multiple regions by considering the process described herein. In one or more implementations, a single classification region may be analyzed to determine the likelihood. In one or more additional implementations, less than all classification regions may be analyzed to determine the likelihood. In various implementations, various combinations of less than all classification regions may be analyzed to determine the likelihood. The modified base caller may determine (e.g., estimate) the tumor fraction that best explains the observed data (e.g., maximizes the likelihood) as an MBD score. To quantify the model confidence, the MBD score may also be defined as the probability that tumor fraction is greater than a cutoff such as 1e-5. A threshold may be applied to the MBD score to generate a binary call on whether ctDNA is present or not.

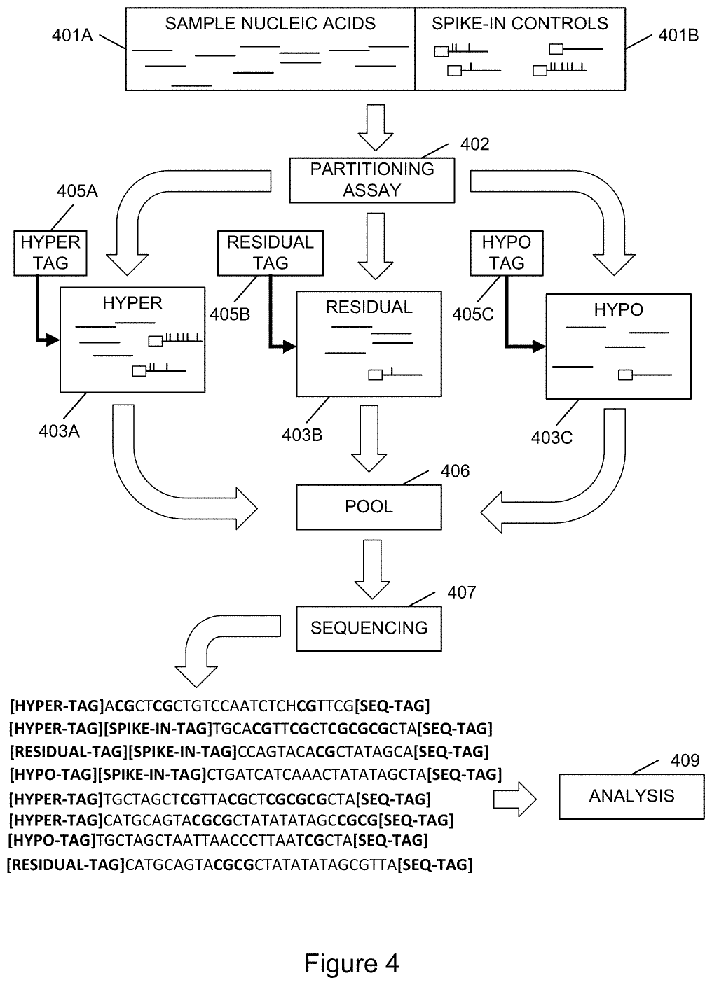

FIG. 4 illustrates illustrative implementations of the disclosure. A sample of different nucleic acids (401A) and spike in control nucleic acids (401B) is partitioned (402) into three different partitions (403A, 403B, and 403C). Each partition (403A, 403B, and 403C) is representative of a different methylation status. Partition 403A comprises a hyper methylation partition, partition 403B comprises a residual methylation partition, and partition 403C comprises a hypo methylation partition. Each partition is distinctly tagged (405A, 405B, 405C). The tag 405A may comprise a hyper methylation tag, the tag 405B may comprise a residual methylation tag, and the tag 405C may comprise a hypo methylation tag. The tagged nucleic acids are pooled together (406) and sequenced (407). Sequencing provides sequence read data (408) that have the sequence of the tag (partition tag, sequence tag, spike-in tag) and the nucleic acid sequence. The sequence read data are analyzed (409). Tags are used to sort sequence reads from different partitions and/or to sort sequence reads of the nucleic acids (401A) from the sequence reads of the control nucleic acids (401B). Analysis (409) results in classification of the sample as tumor-derived or not tumor-derived.

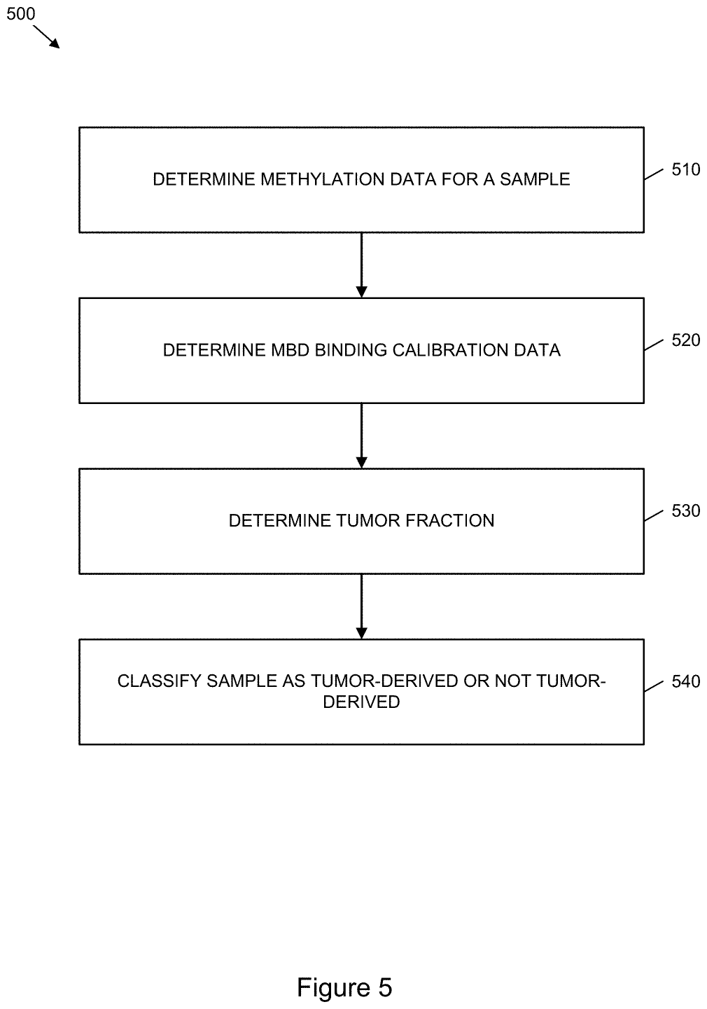

Returning to FIG. 1, the method 100 may comprise classifying the sample as tumor-derived or not tumor-derived at 160. The method 100 may comprise processing the sequence reads using a computational analysis to detect tumor-derived DNA and/or non-tumor-derived DNA at step 160. Such a computational analysis is now described in relation to FIG. 5, which depicts a method 500 of detecting tumor-derived DNA, in accordance with one or more implementations. The method 500 may comprise determining methylation data for sample at step 510, determining MBD binding calibration data at step 520, determining tumor fraction at step 530, and classifying the sample as tumor-derived or not tumor-derived at step 540. One or more features and/or steps of the modified base caller may be modified, removed, or added, based on the type of tumor being detected.

At step 510, methylation data (e.g., the data generated by MBD partitioning) for a sample may be determined. For example, methylation data for one or more classification regions of the sample may be determined. The one or more classification regions may be selected based on the type of disease (e.g., colorectal cancer, lung cancer, etc.) to identify classification regions that are specific to the disease. The one or more classification regions may be selected to include regions that are highly methylated in later stages of a disease and/or in tissues impacted by the disease. The classification regions may be selected based on presence of one or more binding sites (e.g., CTCF binding sites). The one or more classification regions may exclude regions that exhibit high normal methylation rates. In one or more implementations, the one or more classification regions may each comprise a methylation rate that is different from the methylation rates of the other classification regions. In various examples, at least one classification region may comprise a methylation rate that is different from the methylation rates of the other classification regions.

Determining methylation data may comprise receiving and/or requesting sequence read data associated with each sequence read (molecule). The sequence read data may be analyzed to determine the presence of any tags and a number of CG sites per sequence. Any tags detected may be cross-referenced to determine if the tag is associated with a partition (e.g., hyper, residual, hypo), a particular sequence, a particular region, and/or a control sequence. In various examples, an amount of homology can be determined between one or more tags and sequencing reads to identify one or more tags associated with one or more partitions.

The modified base caller is designed to model the modified nucleotide binding reagent (e.g. MBD) partitioning of cfDNA, which is a mixture of tumor derived molecules and normal derived molecules. The methylation data of a sample may comprise millions of molecules. In one or more implementations, the modified base caller sees a molecule as a combination of its partition and the number of CG sites in the molecule (CG count). For example, for a molecule fin the hyper partition and with 10 CG sites, f=(partition,cg count)=(hyper,10). In one or more implementations, the methylation data may comprise one or more of a molecule identifier, a region identifier, a partition identifier, a CG count, combinations thereof, and the like. The molecule identifier may comprise a tag that identifies a sequence read within a partition. The region identifier may comprise a tag that identifies a region of origin for the sequence read with regard to a reference sequence. The partition may comprise an indication of which bin the sequence was binned into. For example, the partition may comprise an indication of “hyper” (highly methylated), “hypo” (lowly methylated), or “residual” (medium level methylation). By way of example, the methylation data may comprise any data structure suitable for storing such data. Example, methylation data may comprise, for example, the data shown in Table 1:

TABLE 1

| Sequence ID | Region ID | Partition | CG Count |
|---|---|---|---|
| 1 | 1 | Hyper | 52 |
| 2 | 1 | Hyper | 63 |
| 3 | 1 | Hypo | 1 |
| 4 | 2 | Hypo | 0 |
| 5 | 2 | Hyper | 34 |
| 6 | 2 | Residual | 1 |

Once the methylation data is obtained, MBD binding calibration may be determined at step 520. In the modified base caller, the source (tumor derived or normal derived) of the molecule may determine whether the methylation rate for that molecule follows the tumor or normal methylation rate distribution, and then the distribution of methylated CG sites and unmethylated CG sites in the molecule. However, since the methylation data provides the observed partition for a molecule (e.g., hyper, residual, or hypo), the modified base caller is configured to accurately calibrate how molecules are grouped based on their methylation pattern (e.g., determine MBD binding calibration data).

In one or more implementations, MBD binding calibration data may be determined according to one or more binding curves. One or more sets of MBD control regions in the panel may be used for this purpose. The one or more sets of MBD control regions may comprise at least one hyper control region and at least one hypo control region. The hyper control region and the hypo control region may be used to generate MBD binding calibration data. The hyper control region may comprise one or more genomic regions that are highly methylated in tumor tissues, normal tissues, and blood cells. In one or more illustrative examples, each CG site in each molecule from these regions are methylated. As a result, the methylated CG count of these molecules can be the same as the total CG count. The hypo control region may comprise one or more genomic regions that are hypomethylated in tumor tissues, normal tissues, and blood cells. CG sites in molecules from the hypo control regions can be fully unmethylated, in one or more examples, and thus the unmethylated CG count equals the total CG count. In one or more illustrative examples, the partitioning pattern of the hyper control molecules can be the same as that of molecules with the same methylated CG count. Similarly, hypo control molecules are partitioned in the same or similar manner as unmethylated molecules with the same unmethylated CG count. In one or more implementations, each classification region may be associated with a respective set of MBD binding calibration data (e.g., methylation binding calibration curves).

Determining MBD binding calibration data may comprise application of one or more binding curves and/or application of a thermodynamic equilibrium model. Determining MBD binding calibration data may comprise determining and/or estimating a methylation rate and/or a methylation rate distribution (used herein interchangeably). Methylation rate may be defined as the probability for a CG site in a molecule to be methylated. In one or more examples, the methylation rates of each CG site in the same molecule can be the same and the methylation of these CG sites can be independent given the shared methylation rate. Moreover, in one or more examples, molecules from the same target regions can share the same methylation rate, which is defined as the methylation rate of the region. Once methylation rates have been determined and/or estimated, given normal methylation rate u, the likelihood of observing a normal derived molecule f, for a certain region, may be determined as P(f|u). Similarly, given tumor methylation rate v, the likelihood of observing a tumor derived molecule f, for a certain region, may be determined as P(f|v).

In one or more implementations, the normal methylation rate may be flat (e.g., uniform distribution between 0 and 1), meaning that no assumption is made about the methylation rate of normal derived molecules. In one or more additional implementations, the normal methylation rate may be determined based on methylation data of a disease-free sample. For example, the probe/region-level normal methylation rate can be estimated based on methylation data of a cancer-free sample. The likelihood of the normal methylation rate may be determined given known zero tumor fraction (θ=0) because 0 tumor derived molecules are expected in disease-free samples. With 0 tumor fraction, the likelihood becomes:

$$P ( {D}_{i} \vert θ = 0 , u , v ) = {\prod }_{f ∈ {D}_{i}} P ( f \vert u )$$

Normalizing the likelihood to a probability distribution, can determine the normal methylation rate posterior distribution as:

$$P ( u = {u}_{k} \vert {D}_{i} ) = \frac{P ( u = {u}_{k} ) {\prod }_{f ∈ {D}_{i}} P ( f \vert u = {u}_{k} )}{{\int }_{0}^{1} P ( u ) {\prod }_{f ∈ {D}_{i}} P ( f \vert u ) d u}$$

For a given region, the aggregate of different normal methylation rate posterior distributions in a set of disease-free samples can be used to estimate the prior distribution of normal methylation rate and used as input to the modified base caller. In one or more implementations, classification regions may comprise the same prior distributions of methylation rates. In various implementations, classification regions may comprise differing prior distributions of methylation rates. In one or more implementations, classification regions that comprise the same, or differing prior distributions of methylation rates, may be analyzed in the same experiment. In addition, the expected normal methylation rate can be calculated for each probe/region as:

$${E}_{i} ( u ) = {\int }_{0}^{1} u P ( u \vert {D}_{i} ) d u$$

which may be used for filtering out probes/regions that are highly methylated in cancer-free samples. In one or more implementations, the expected normal methylation rate for each sample for each probe may be determined, and then mean+two-fold standard deviation of the expectations may be determined, on which a cutoff is applied to filter out probes with a high normal methylation rate.

In various implementations, the one or more sets of MBD classification regions may be filtered to remove confounding methylation signals from normal tissues and blood cells. As described, the modified base caller is configured to detect ctDNA by distinguishing tumor derived hypermethylation signatures from normal derived hypomethylation signatures on classification regions. In one or more instances, tumor derived hypermethylation can be confounded by hypermethylated molecules from non-tumor sources (e.g., blood and normal tissues). To reduce the impact of such confounding factor, whole-genome bisulfite sequencing (WGBS) data of blood cells and normal tissues may be determined, and molecules that may contain nontumor derived hypermethylation patterns may be filtered out.

In one or more implementations, a mean blood methylation rate mᵢ^{blood} may be determined for each CG site l in the genome as the average methylation rate of the given CG site in all blood cell types. A mean normal tissue methylation rate mᵢ^{tissue} may be determined for each CG site l based on the WGBS data of a set of normal tissues. The methylation rate of a CG site may not be available in all blood cell types or normal tissues surveyed and only sites supported by at least a given number of cell types or normal tissues may be considered, where the mean blood/tissue methylation rate is confidently estimated.

Next, for a molecule f that overlaps with a set of CG sites l∈L_{f}, an expected methylated CG count if the molecule is from blood or normal tissue may be determined as:

$${\sum }_{l {inL}_{f}}^{ } {m}_{l}^{blood}$$

$$and$$

$${\sum }_{l {inL}_{f}}^{ } {m}_{l}^{tissue}$$

One or more cutoff(s) (e.g. 1.0) may be applied to these two values. Molecules with overly high expected methylated CG count may be filtered out because these molecules potentially confound the modified base caller especially in samples with high normal tissue/blood derived fraction.

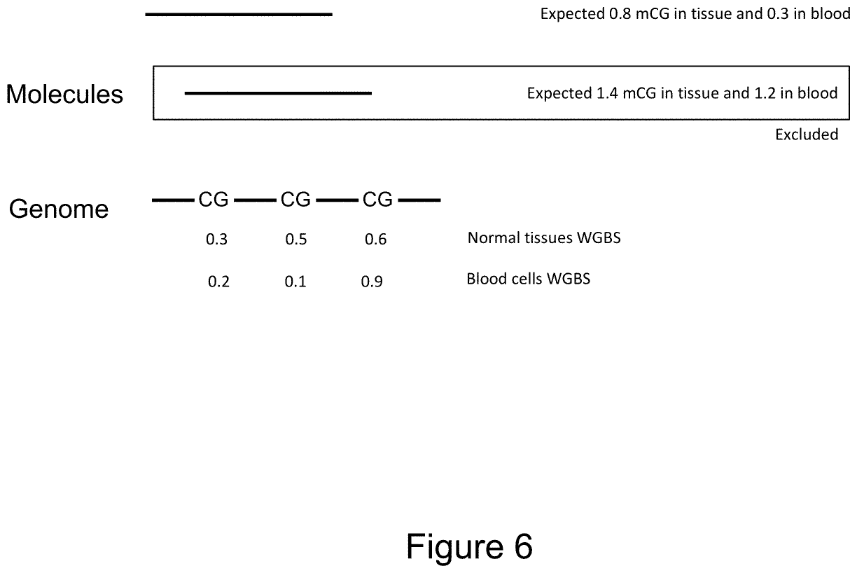

FIG. 6 shows an example where a cutoff at 1.0 is used to filter two molecules. The first molecule is overlapped with two CG sites covered by WGBS data, based on which the expected methylated CG count in normal tissue and in blood cells can be computed as 0.8 and 0.3. Since both values are below cutoff, the first molecule is kept. The same filter is applied to the second molecule and it is filtered out due to high expected methylated count in both blood cells and normal tissues.

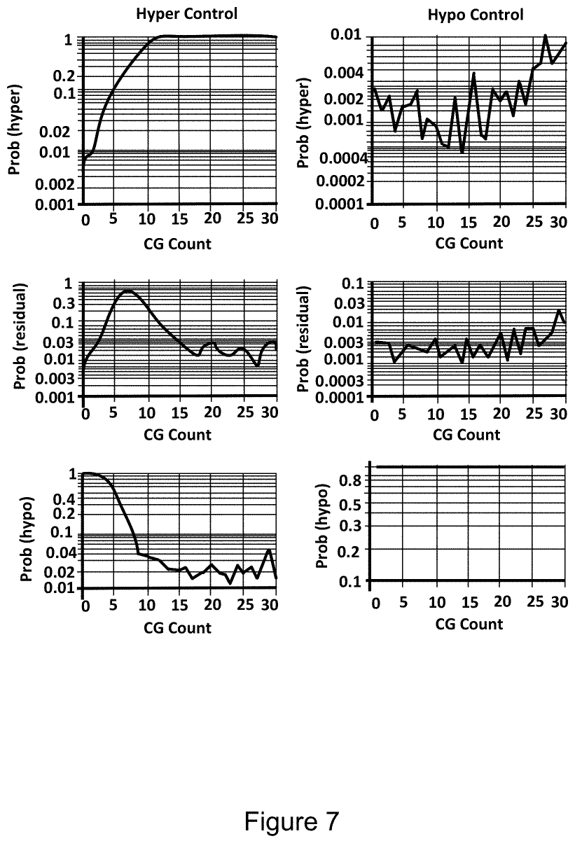

MBD binding calibration data obtained from the control regions can be visualized using MBD binding curves (calibration curves). An example is shown in FIG. 7. The left three curves are based on molecules from hyper control regions and they calibrate the probability for a molecule with a given number of methylated CG sites to enter each partition. The three curves on the right are estimated using molecules from hypo control regions, which calibrate the probability for a molecule without methylated CG but with a given number of unmethylated CG sites to enter each partition. The scale of impact of methylated CG sites is one to three orders of magnitude larger than that of unmethylated CG sites and therefore, the effect of unmethylated CG sites is not calibrated for molecules with any methylated CG site(s).

In one or more implementations, the MBD calibration curves may be directly estimated from the partitioning of molecules associated with the one or more MBD control regions. For example, the probability for a molecule with 2 methylated CG sites to enter the hyper partition may be estimated as the hyper fraction of 2 CG hyper control molecules. Assuming that there are 1,0002 CG site hyper control molecules, 10, 5, and 985 of which are in hyper, residual, and hypo partitions, respectively, then the probability may be estimated as 10/1000=0.01 (which is also called “naive fraction”).

Application of one or more MBD binding curves may comprise using the one or more MBD binding curves to estimate the probability that MBD partitions a molecule with a given number of methylated CG sites or a given number of unmethylated CG sites. FIG. 7 shows an example of MBD binding curves, a representation about MBD partitioning, which is estimated from the partitioning data of molecules from control regions. In the MBD binding curves, the CG count of molecules from hyper control regions indicates the number of methylated CG sites, whereas that of molecules from hypo control regions corresponds to the number of unmethylated CG sites. The molecules from other target regions in the panel are expected to be partitioned in the same way.

For a molecule with m methylated CG sites out of c total CG sites, P^{hyper} (b|c) is the probability for this molecule to enter partition b if m is not zero. P^{hyper} (b|c) is the curve estimated from hyper control data. If m is zero, then the probability is P^{hypo} (b|c), which corresponds to the curves estimated based on hypo control data.

For a molecule with m methyated CG sites out of c total CG sites, P^{hyper} (b|c) is the probability for this molecule to enter partition b if m is not zero. P^{hyper} (b|c) is the curve estimated from hyper control data. If m is zero, then the probability is P^{hypo} (b|c), which corresponds to the curves estimated based on hypo control data.

For a molecule f=(b,c) in partition b, with c CG sites and with methylation rate r, the likelihood of observing this molecule is P(f|r)=P(b|r, c). The number of methylated CG sites, m, follows a binomial process with total trial number c and success probability r. Therefore,

$$P ( b \vert r , c ) = {P}^{binom} ( 0 \vert c , r ) {P}^{hypo} ( b \vert c ) + {\sum }_{m = 1}^{c} {P}^{binom} ( m \vert c , r ) {P}^{hyper} ( b \vert m )$$

where P^{binom}(m|c,r) is the binomial probability of m success out of c trials given success probability r.

As described above, modified base caller depends on the partitioning data of molecules from hyper and hypo control regions to calibrate MBD partitioning patterns, i.e. to estimate P^{hyper} (b|m) and P^{hypo} (bκ). In an embodiment, P^{hyper} (b|m) may be estimated by counting the fraction in partition b of molecules that have m CG sites and are from hyper control regions, for non-zero m. In an embodiment, P^{hypo} (b|c) may be estimated as the fraction in partition b of molecules that have c CG sites and are from hypo control regions.

Specifically, let n_{b,c}^{hyper} be the number of molecules in partition b, with c CG sites and from hyper control regions. Similarly, let n_{b,c}^{hypo} be the number of molecules in partition b, with c CG sites and from hypo control regions. Then,

$${p}^{hyper} ( b \vert c ) = \frac{{n}_{b , c}^{hyper}}{{n}_{hyper , c}^{hyper} + {n}_{residual , c +}^{hyper} + {n}_{hypo , c}^{hyper}}$$

$$and$$

$${p}^{hypo} ( b \vert c ) = \frac{{n}_{b , c}^{hypo}}{{n}_{hyper , c}^{hypo} + {n}_{residual , c +}^{hypo} + {n}_{hypo , c}^{hypo}}$$

In practice, however, n_{b,c}^{hyper} and n_{b,c}^{hypo} could be zero, resulting in probabilities P^{hyper} (b|m) and P^{hypo}(b|c) being 0.0. It is unreasonable and risky to assume that a certain molecule has 0 chance to enter a particular partition because there is technical noise in the MBD assay. Also, 0 probability could be due to insufficient molecule counts. For example, it is likely to observe 0 hyper molecules if only sequence 1000 molecules were sequenced and the true probability for entering hyper partition is 1e-5.

In an embodiment, a pseudocount may be implemented to address this issue. The pseudocount may add one molecule count to n_{b,c}^{hyper} and n_{b,c}^{hypo}. With pseudocount, the equations for MBD binding curves become:

$${P}^{hypo} ( b \vert c ) = \frac{{n}_{b , c}^{hyper} + 1}{{n}_{hyper , c}^{h y p e r} + {n}_{residual , c +}^{h y p e r} + {n}_{hypo , c}^{hyper} + 3}$$

$$and$$

$${P}^{hyper} ( b \vert c ) = \frac{{n}_{b , c}^{hypo} + 1}{{n}_{hyper , c}^{hypo} + {n}_{residual , c +}^{hypo} + {n}_{hypo , c}^{hypo} + 3}$$

These equations will avoid 0 probability and result in conservative estimates of the P^{hyper} (b|m) and P^{hypo} (bκ) especially when these probabilities are very small and number of molecules used for estimation is small. In the previous example, the hyper probability estimate become ˜1e-4 (1/1003) with pseudocount compared to 0 (0/1000) without pseudocount.

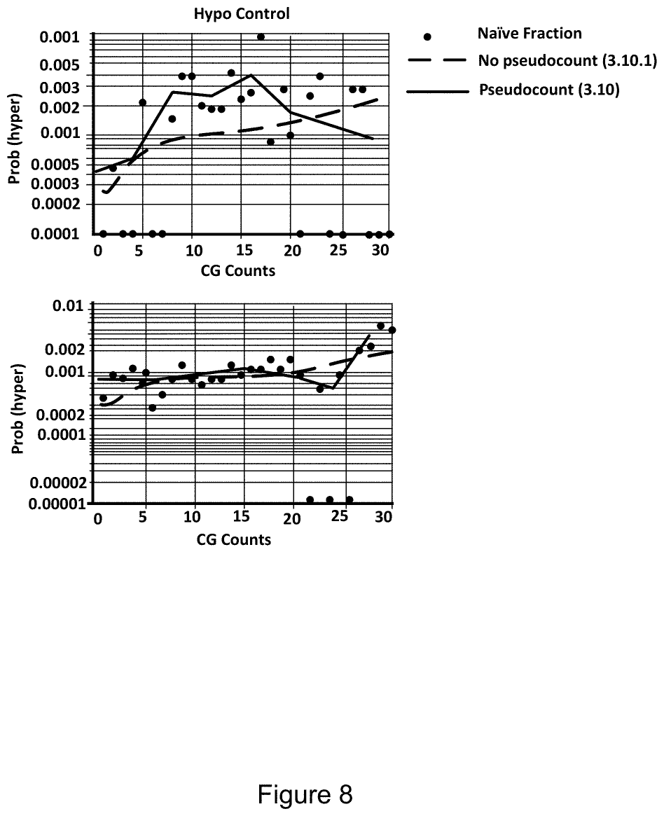

In one or more examples, utilizing 1e-4 vs 1e-5 could impact the results generated using a pseudo-count (e.g., pseudocount could lead to overestimation of a small probability). In these situations, the estimation of the probability of unmethylated molecules to enter hyper or residual partitions (e.g., hypo control binding curves) may be impacted because these probabilities are on the scale of 1e-4 and sometimes even 1e-5. In various examples, the modified base caller, configured with such parameters, may overestimate the noise (unmethylated molecules in hyper/residual partition) level and become overly conservative. Hypo control calibration curves are most affected by this overestimation. In one or more examples, use of pseudocount to level the hypo control calibration curves can make the modified base caller overly conservative. The overestimation may be addressed by first estimating the “shape” of the hypo control calibration curves and then “anchoring” the curves by moving the curves up and down (in log scale) until the curves best fit the data. FIG. 8 shows an example of hypo control calibration curves, where the blue points show the naive fraction. Red lines are the curves estimated using pseudocount, whereas orange curves are estimated using no pseudocount. There are many points with zero probability based on naive fraction. These points may be used to drive the estimation of the binding curves using pseudocount and the final estimated binding curves (red lines) are spiky. In comparison, the estimation without pseudo count is more robust and the binding curves (orange lines) are smoother. Also, the binding curves are expected to be monotonic since MBD affinity should increase for unmethylated molecules with more CG sites. In various examples, the binding curves without pseudocount can fit better with the expectation.

For the hyper control calibration curves, the impact of pseudocount may be very small if not negligible because the hyper/residual probabilities are large enough to tolerate the use of pseudocount. Therefore, pseudocount may be used for estimating the hyper control binding curves and not the hypo control binding curves.

Computationally, to reduce the impact of pseudocount, the number molecules used for estimating P^{hypo} (b|c) may be increased by pooling the data of molecules with similar CG count. For example, molecules with a CG count between 0 and 3, 4 and 7, etc. share the same P^{hypo}(b|c). In one or more additional implementations, to reduce the impact of pseudocount, the hypo control binding curves may be estimated without using the pseudocount. In various examples, the shapes of the two curves representing the probability of unmethylated molecules with different CG counts to enter hyper and residual partition can be identical or similar in log scale for different samples. In these scenarios, the curves of different samples may differ by a scaling factor. Specifically, the probability for unmethylated molecules with c CG sites to enter partition b in sample s is:

Pₛ^{hypo}(b|c)=Cₛ^{b}f(b,c)

where partition b∈{“hyper”, “residual”}. f(b,c) is a function for defining the curve shape and it is the same for all samples. Cₛ^{b} is a sample-specific scaling factor, which scales the fixed curves f(b,c) to fit the partitioning of molecules from hypo control regions.

In addition, there are constraints on the two curves (probability of unmethylated molecules to enter hyper or residual partitions), which can allow better curve estimation. Due to the properties of the MBD protein, the curves are monotonic and continuous. First, unmethylated molecules with more CG sites have higher chance to enter hyper or residual partition: Pₛ^{hypo}(b,c)≤Pₛ^{hypo} (b|c+1)≤Pₛ^{hypo} (b|c+2) for b∈{“hyper”, “residual”}. Second, the difference between Pₛ^{hypo} (b|c) and Pₛ^{hypo} (b|c+1) is due to an additional unmethylated CG, which should be smaller than the effect of an additional methylated CG. As a result, Pₛ^{hypo} (b|c) and Pₛ^{hypo} (b|c+1) are expected to be very similar. Therefore, the two curves are expected to be smooth, which can lead to the use of a smoothing technique to improve the estimation.

To determine the shape of the hypo control binding curves (f (b,c)), the curves from a set of cancer-free samples may be aggregated. Then, select the Cₛ^{b} to best fit the MBD binding calibration data obtained from the control regions. Specifically, the hyper control regions are assumed to be fully methylated (methylation rate=0.9999999999) and the hypo control regions are assumed to be unmethylated (methylation rate ˜1e-4). Then, the likelihood of the observed control methylation data may be determined as:

$$P ( D ) = {\prod }_{( b , c ) ∈ D}^{ } [ {p}^{binom} ( 0 \vert r , c ) * {C}_{s}^{b} * f ( b , c ) + {\sum }_{m = 1}^{c} ( {p}^{binom} ( m \vert r , c ) * {p}^{hyper} ( b \vert m ) ) ]$$

where D is the collection of all molecules from the MBD control regions. r is the methylation rate of either hyper control regions or hypo control regions. R is set to 1e-4 for molecules that are from hypo control regions, while r is set to 0.9999999999 for molecules from hyper control regions. P^{hyper} (b|m) is the hyper control binding curve (See FIG. 7). L-BFGS algorithm can be used to select the Cₛ^{b} that maximizes the likelihood given Σ_{b∈{hyper,residual,hypo}}Cₛ^{b} (b,c)=1 for any CG count C.

### Application of a Thermodynamic Equilibrium Model

In one or more additional implementations, rather than utilizing MBD binding curves, the MBD partitioning data may be determined according to a thermodynamic equilibrium approach. Accurately calibrating MBD partitioning is relevant to accurate detection of ctDNA from methylation data. In some examples, it may not be desirable to empirically estimate the partitioning probability for molecules with given number of methylated and unmethylated CG (mCG) sites based on the molecules from endogenous control regions according to MBD binding curves. For example, the partitioning probability estimated for molecules with 1 and 2 mCG sites may not be sufficiently accurate and such inaccuracy could negatively impact modified base caller performance (e.g., clinical sensitivity, specificity and analytical limit of detection (LoD)).

Accurate estimation of the partitioning probability of each possible number of mCG may be vulnerable to various types of noise. For example, for the molecules from hyper control regions and with 1 CpG site, it is expected that the molecules to be fully methylated and contain exactly 1 mCG site. In various examples, though, the methylation rate is unlikely to be 100% and also there may be non-CpG methylation. As a result, the partitioning pattern reflects the partitioning pattern more akin to 1.3 mCG sites. Additionally, as the probability of these molecules to enter the hyper partition is small (e.g., 1e-3), the estimation of the partitioning probability may be impacted by sampling variation (e.g., count noise) whereas accurate estimation may rely on a large number of molecules.

In one or more examples (e.g., for molecules containing a low number of CG sites), a thermodynamic model may be used to determine the MBD binding calibration data). Compared to empirical estimation, in at least some instances, the thermodynamic model can require fewer parameters and allows co-estimating the probability of at least a portion of mCG points. In various examples, the thermodynamic model may utilize information from other mCG points to impute one or more mCG points that may be associated with inaccuracies.

In one or more implementations, a two-step model may be used to determine the MBD binding calibration data. The first step corresponds to the first two (MBD) washes to obtain the hypo partition. It is assumed that the binding energy is proportion to the number of methylated CG sites (x) and it is equal to x*e_{bound}. The two-step model thus utilizes four parameters and the probability of three partitions sums to 1. The probability for a molecule with methylated CG sites to bind or not bind MBD is”

$$P ( bound ) = \frac{exp ( x * {e}_{bound} )}{exp ( x * {e}_{bound} ) + exp ( {e}_{unbound} )}$$

$$and$$

$$P ( unbound ) = \frac{exp ( {e}_{unbound} )}{exp ( x * {e}_{bound} ) + exp ( {e}_{unbound} )}$$

Molecules that are not bound will fall into the hypo partition. Therefore:

P(hypo)=P(unbound)

The second step corresponds to the next two washes to obtain the residual partition. Since only the MBD bound molecules will go through this step of partitioning, the probability for a molecule to enter the hyper partition is the product of the probability that the molecule is bound in the first step and the probability that it is bound in the second step:

$$P ( hyper ) = P ( bound ) * \frac{exp ( {e}_{hyper} )}{exp ( x * {e}_{hyper} ) + exp ( {e}_{residual} )}$$

The molecules that are bound in the first step but not in the second step fall into residual:

$$P ( residual ) = P ( bound ) * \frac{exp ( {e}_{residual} )}{exp ( x * {e}_{hyper} ) + exp ( {e}_{residual} )}$$

In one or more examples, the two-step model may be modified by accounting for explicit noise. An issue of the two-step model is that, P(hypo) may be very close to zero if x is high. In various examples, a relatively small number molecules with a large number of methylated CG sites (x) may be placed into the hypo partition. This small fraction may be considered as noise to the modified base caller. Accordingly, a modified version of two-step model is described that takes into account the noise. Assuming the degree of noise is the same for all three partitions, a parameter bₙₒᵢₛₑ may be used to represent noise level. This parameter may be added to the probability for the three partitions from the two-step model and then the probabilities may be renormalized to sum to 1. This model thus utilizes five parameters.

$${P}^{′} ( hyper ) = \frac{P ( hyper ) + {b}_{noise}}{P ( hyper ) + P ( residual ) + P ( hypo ) + 3 * {b}_{noise}}$$

$${P}^{′} ( residual ) = \frac{P ( residual ) + {b}_{noise}}{P ( hyper ) + P ( residual ) + P ( hypo ) + 3 * {b}_{noise}}$$

$${P}^{′} ( hypo ) = \frac{P ( hypo ) + {b}_{noise}}{P ( hyper ) + P ( residual ) + P ( hypo ) + 3 * {b}_{noise}}$$

In one or more implementations, the two-step model may be modified to use effective methylated CG count instead of methylated CG count. For MBD to bind molecules with large numbers of methylated CGs, the molecules have to bend to some degree and effectively, the binding affinity does not grow linearly with the number of methylated CGs. To account for this, the effective methylated CG count x′ may be used and it may be assumed that the binding energy is increased linearly to the effective methylated CG count x′. A quadratic term may be defined to account for the difference between the methylated CG count and the effective methylated CG count. The effective methylated CG count may vary between the two steps: x′=x+α*x² and x″=x+β*x². This model thus utilizes six parameters. The calculation of probability becomes:

$$P ( hypo ) = P ( unbound ) = \frac{exp ( {e}_{unbound} )}{exp ( {e}_{unbound} ) + exp ( {x}^{′} * {e}_{bound} )}$$

$$P ( residual ) = P ( bound ) * \frac{exp ( {e}_{residual} )}{exp ( {e}_{residual} ) + exp ( {x}^{″} * {e}_{hyper} )}$$

$$P ( hyper ) = P ( bound ) * \frac{exp ( {x}^{″} * {e}_{hyper} )}{exp ( {e}_{residual} ) + exp ( {x}^{″} * {e}_{hyper} )}$$

$$where$$

$$P ( bound ) = \frac{exp ( {x}^{′} * {e}_{bound} )}{exp ( {e}_{unbound} ) + exp ( {x}^{′} * {e}_{bound} )}$$

In one or more additional implementations, a linear regression-based method to determine the MBD binding calibration data. In particular, MBD binding calibration associated with molecules containing low numbers of methylated CG sites. While the model with quadratic term gives the best fit, there is no guarantee of convergence of its parameters. In order to impute the 1 mCG and 2 mCG points, the continuity of the binding curves may be utilized and interpolation may be performed rather than model fitting. In various examples, the 0, 3, 4 and 5 mCG points may fall in a line in log-space and linear regression may be used to estimate the 1 and 2 mCG points. Interpolation and linear regression tend to fit the data well and provide reasonable estimates of the 1 and 2 mCG points.

Returning to FIG. 5, once the MBD binding calibration data is determined at step 520, the method 500 may proceed to determine a tumor fraction at step 530. For a certain region, given normal methylation rate u, the likelihood of observing a normal derived molecule f may be determined as P(f|u). Similarly, given tumor methylation rate v, the likelihood of tumor derived molecule may be determined as P(f|v). For a collection of molecules from a certain region, a certain fraction (0.0 to 1.0) of the molecules are from tumor cells. The fraction is referred to as tumor fraction, denoted as θ. For a molecule with unknown source, with no knowledge of the molecule's partition and CG count, the probability for the molecule to be tumor derived will be θ and the probability for the molecule to be normal derived will be 1−θ. Therefore, considering the possibilities that a molecule with unknown source can be either tumor derived or normal derived, the likelihood of observing molecule f may be determined as:

P(f|θ,u,v)=(1−θ)P(f|u)+θP(f|v)

The above equation is for one molecule from a given region. The likelihood for all molecules from the given region may also be determined. Given a normal methylation rate and a tumor methylation rate, all molecules from that region are independent because their CG counts are fixed and then the molecule partitioning is only dependent on the methylation rate. So, for all molecules Dᵢ from a given region i, the likelihood of observed data given tumor fraction θ, normal methylation rate u, and tumor methylation rate v is:

$$P ( {D}_{i} \vert θ , u , v ) = {\prod }_{f ∈ {D}_{i}}^{ } [ ( 1 - θ ) P ( f \vert u ) + θ P ( f \vert v ) ]$$

This equation enables fitting methylation data with a known θ, u, and v.

In one or more examples, the normal methylation rate u and tumor methylation rate v may be determined from the MBD binding calibration data from step 520, as described below. In various examples, a binomial distribution can relate the MBD binding calibration data to a methylation rate parameter, and, in these scenarios, the MBD binding calibration data can be used to define a likelihood over a range of methylation rates. In order to infer u and v together, tumor fraction θ (the relative mixture between tumor and normal fragments) may be inferred or marginalized out.

In one or more implementations, where the MBD binding calibration data was determined based on classification target regions, the tumor methylation rate may be relatively high and the normal methylation rate may be relatively low. The likelihoods for all possible normal methylation rates and tumor methylation rates may be determined and summed, and a relatively large weight may be assigned for methylation rates having a relatively higher likelihoods and a relatively small weight may be assigned for methylation rates having relatively lower likelihoods. In various implementations, the methylation rate or normal, non-tumor cells may be relatively constant. In one or more examples, the tumor methylation rate may be 1.0 or close to 1.0 such that highly methylated molecules may be considered as tumor derived.

By incorporating the normal methylation prior P(u) and tumor methylation rate prior P(v), the likelihood of observed data for region i given tumor fraction θ may be determined as:

$$P ( {D}_{i} \vert θ ) = {\int }_{u} {\int }_{v} P ( u ) P ( v ) {\prod }_{i}^{ } [ ( 1 - θ ) P ( f \vert u ) + θ P ( f \vert v ) ] dudv$$

In one or more examples, the normal methylation rates and the tumor methylation rates from different regions can be independent. In these situations, the likelihood of observed data across multiple regions may be determined as:

$$P ( D \vert θ ) = {\prod }_{i}^{ } P ( {D}_{i} θ )$$

In various examples, methylation rates of normal cells and methylation rates of tumor cells can be independent due to DNA methylation operating on a small genomic interval. In one or more additional examples, genomic regions that are >1 kb away, may be methylated or unmethylated together due to physical proximity or co-regulation by the same pathway and may be more likely to be regulated independently.

Next, the posterior probability distribution of tumor fraction θ may be determined as:

$$P ( θ = {θ}_{j} \vert D ) = \frac{P ( θ = {θ}_{j} \vert D )}{{\int }_{θ} P ( θ \vert D )}$$

In one or more implementations, the posterior probability distribution of tumor fraction θ may be used to determine a posterior mean tumor fraction. Once the posterior probability distribution of tumor fraction θ, P(θ|D), is determined, the posterior mean may be determined as the posterior-weighted average of θ values: ∫θ*P(θ|D).

At step 540, the sample may be classified as tumor-derived or not tumor-derived. In an embodiment, an MBD score may be defined as the probability that tumor fraction θ is greater than a certain threshold. The threshold may be, for example, from, and including, about 1e−10 to about 1e-5. Other thresholds are contemplated as described below.

P(θ≥10⁻⁵|D).

In an embodiment, an MBD score may be defined as the posterior mean tumor fraction. The posterior mean tumor fraction may be compared to a threshold to classify the sample as tumor-derived or not tumor-derived. The threshold may be, for example, from, and including, about 3e-4 to about 6e-4. Other statistical measures are contemplated for use as an MBD score.

A “high” MBD score can indicate at least threshold amount of confidence that the tumor fraction is not zero and the sample may be classified as tumor-derived whereas a “low” MBD score can indicate less than an additional threshold amount of confidence that the tumor fraction is zero and the sample may be classified as not tumor-derived.

Other thresholds are contemplated and may vary depending on MBD partitioning calibration, selection of classification regions, filtering on molecules with non-tumor hypermethylation signals, and determination of normal methylation rate prior, all as described above. For example, adjustments in the estimation of MBD binding curves may result in causing the processes and systems described herein to be more sensitive and more aggressive, resulting in an increased MBD score for normal samples. Similarly, selecting classification regions with high tumor methylation rate and using a data-derived normal methylation rate prior instead of a flat prior normal methylation rate can also increase the sensitivity of the processes and systems described herein. In one or more examples, these scenarios can cause the processes and systems described herein to be more sensitive to assay noise and biological noise, resulting in an increased MBD score for normal samples. In contrast, the addition of filtering on molecules with non-tumor hypermethylation signals will decrease the MBD score for normal samples. Therefore, the threshold for the MBD score in relation to making a call that the sample is from a subject in which a tumor is present may be adjusted to balance clinical sensitivity and specificity based on the features and/or steps performed by the systems and methods described herein. In one or more implementations, to further increase clinical specificity, the tumor methylation rate prior may be slightly modified.

In some implementations, the methods and systems described herein can be used to determine drug or therapeutic sensitivity. For example, the methods and systems described herein can be used to detect homologous recombination deficiency (HRD) by analyzing the promoter regions of homologous recombination repair (HRR) genes like BRCA1, BRCA2. In some implementations, the HRD can be used as a biomarker for response to PARP inhibitors.

The following examples illustrate the present methods and systems as they relate to colorectal cancer detection. The following Examples are not intended to be limiting thereof.

### INTRODUCTION

The addition of plasma circulating tumor DNA (ctDNA) to treatment decision making for patients with advanced cancer has improved adherence to guideline recommendations for genomic testing, decreased wait time for results, improved cost and assignment of appropriate therapies, and reduced adverse events due to biopsy related procedures and treatment with ineffective therapies. The adoption of this technology is due in part to the non-invasive nature of a blood-based test, as compared to the prior standard of care, invasive tissue biopsy.

Research efforts continue to indicate that ctDNA can be utilized in earlier stage cancers—to assess for minimal residual disease (MRD) following a therapeutic intervention to determine patients who may benefit from additional therapies, for longitudinal monitoring of disease recurrence, and for cancer detection in asymptomatic individuals.

Demonstrating the clinical utility of ctDNA within the screening paradigm presents novel challenges: significantly lower tumor cfDNA fractions; and the increasing relevance of biologic confounders (e.g. clonal hematopoiesis of indeterminate potential (CHIP)). To effectively utilize ctDNA technology requires expansion beyond genomic assessment of somatic alterations to incorporate assessment of epigenomic signals to significantly improve sensitivity.

A blood based ctDNA assay was developed that utilizes a multimodal approach to colorectal cancer detection: identification of tumor derived genomic alterations, and epigenomic modifications due to differential methylation as well as nucleosomal positioning resulting in differential cfDNA fragmentation patterns.

Methods

Sample Collection and Plasma Isolation

All subjects provided written informed consent for collection of peripheral venous blood. Cases were prospectively recruited following confirmation of a CRC diagnosis by standard clinical procedures. Blood was collected from 173 subjects prior to resection of the primary tumor. Controls were prospectively recruited and confirmed by screening colonoscopy to be negative for advanced neoplasia. Blood was collected from 191 subjects pre-colonoscopy. An additional set of 208 self-reported cancer free individuals were recruited for participation and blood collection. Across all cohorts, blood was collected into two to four 10 mL Streck Cell-Free DNA blood collection tubes (Streck, Inc.). Age, gender, and cancer stage (as appropriate) was collected for each subject. Plasma was isolated according to Guardant Health standard operating procedures for immediate cfDNA extraction or stored at −80 C. Frozen plasma was thawed at room temperature prior to extraction or overnight at 4° C.

Panel Design

A 500 kb panel that targets both common oncogenic mutations, as well as regions expected to undergo epigenomic modification in cancer was developed utilizing an extensive literature review, ctDNA sequencing results from over 100,000 patients with advanced cancer who underwent testing using a blood-based cfDNA assay, whole genome sequencing results from 65 self-reported healthy donors and 48 patients with advanced solid tumors.

cfDNA Extraction, Library Preparation, Sequencing, and Bioinformatics Analysis

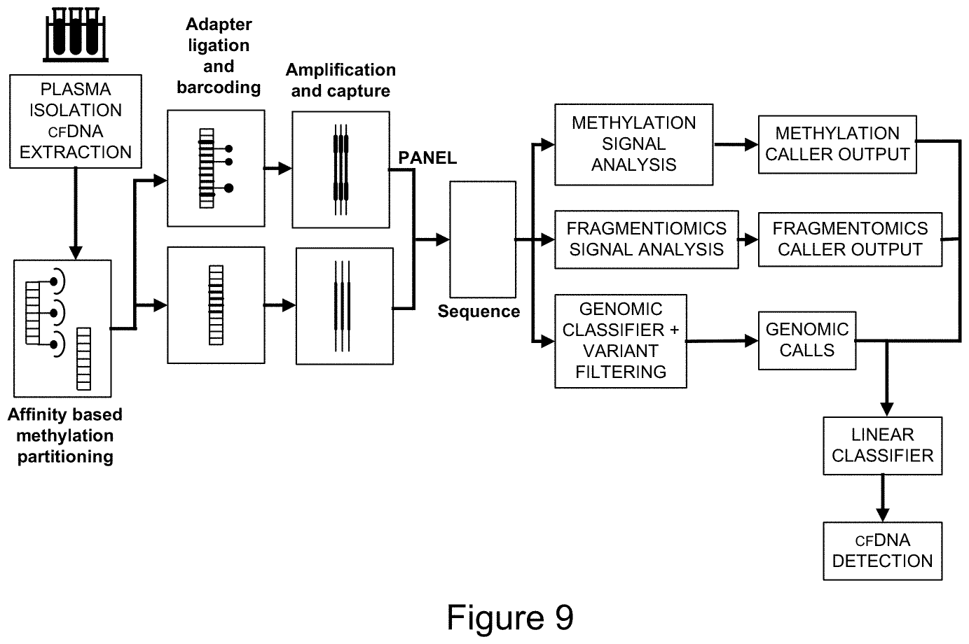

The laboratory and bioinformatic workflow are summarized in FIG. 9. Briefly, cell free DNA (cfDNA) is extracted from plasma in an automated process on the QIAsymphony platform using the QIAsymphony DSP Circulating DNA Kit (Qiagen). Up to 150 ng of the extracted cfDNA is then used as input to the ctDNA assay methylation partitioning and library preparation workflow, which is automated on Microlab STAR (Hamilton) platform. Briefly, the extracted cfDNA undergoes affinity-based partitioning based on the level of methylation in the cfDNA molecule. Individual molecules within each partition are then ligated using adapters that contain non-random partition-specific barcodes, after which partitions are recombined and processed through library preparation. The libraries are enriched using the 500 kB panel (described above) using biotinylated bait oligonucleotides, indexed, pooled and sequenced on a NovaSeq 6000 System.

Sequencing reads are analyzed to extract molecular barcodes mapping each of the reads to an individual molecule and methylation partition, and then aligned to the human genome (hg19). Three independent analyses are performed in parallel: (1) detection of somatic mutations and variant filtering to differentiate somatic mutations associated with CRC tumors from variants associated with other processes such as clonal hematopoiesis of indeterminate potential and (2) assessment of the observed distribution of cfDNA molecules across different methylation partitions within pre-selected regions determined to be differentially methylated in CRC tumors compared to cellular component of whole blood; (3) assessment of cfDNA fragmentation patterns in genomic regions across the panel. The signals from all three analyses are combined using a linear classifier to produce a continuous tumor presence score.

Somatic Variant Filtering

Somatic variant filtering was developed to differentiate tumor derived alterations from those variants likely originating from clonal hematopoiesis of indeterminate potential (CHIP) and frequently found in cfDNA at a range of allelic fractions. Data from over 100,000 late stage cancer patient samples processed from plasma with targeted sequencing using a blood based cfDNA assay was analyzed to assess characteristics associated with variants frequently found in the clonal allele frequency range. It was hypothesized that such mutations are substantially more likely to be of tumor origin. Using this logic, a score was generated per mutation that describes the confidence that this mutation is associated with the tumor. Using this score, a calling threshold and variant selection rules were defined prospectively and used this filter and likely to be associated with tumor presence with high confidence.

Methylation Analysis

Using a set of positive and negative (ubiquitously hyper- or hypo-methylated in most tissue types respectively) regions the behavior of the methylation partitioning was calibrated per sample. Using this calibration and a Bayesian model a score was generated per region describing the likelihood of the observed methylation patterns in this region given a specific tumor fraction. A sample-level estimate of tumor fraction was then generated by combining these likelihoods across all the regions in the panel designed to capture differential methylation.

cfDNA Fragmentation Pattern Analysis

Using a set of both healthy donor and cancer patients a number of different models were trained to capture the statistical relation between the differential coverage patterns per position and tumor presence. These scores were then summarized per region and integrate the scores from all the panel regions into one prediction for model. Finally, an ensemble classifier was used to integrate all model predictions to the final fragmentation score.

Estimation of Tumor Fraction from Methylation Partition Analysis

Tumor fraction estimate based on the analysis of methylation partition origin of cfDNA molecules is defined as the maximum posterior estimate of this parameter in the generative Bayesian model defined to explain the observed distribution of molecules across differentially methylated regions.

Normalizing Methylation Percentage for Visualization of Signal

To visualize the methylation signal per region X(s,r) was defined as the percentage of cfDNA molecules in region r partitioned to the hyper-methylated bin in sample s. To normalize this percentage, neg(s) was defined as the average percentage of molecules in the hyper-methylated bin in a set of ubiquitously hypo-methylated regions in sample s and respectively pos(s) as the average percentage of molecules in the hyper-methylated partition in a set of ubiquitously hyper-methylated regions in sample s. Finally, the normalized percentage of reads was defined per region as: [X(s,r)−neg(s)]/[pos(s)−neg(s)].

Training Set

Samples from 204 individuals were identified for inclusion in the training set (Table 2). These samples were used for three main purposes. They were used to (a) estimate the parameters of the somatic mutation detection, methylation, and fragmentation analysis methods; (b) train the linear classifier integrating the results of the individual callers into a single continuous predictor; (c) establish a tumor presence prediction threshold over the linear classifier output for binary prediction of overall sample status targeting less than 5% false positive rate.

Results

The model performance was tested on a blinded cohort of samples collected from 368 subjects including those with CRC (N=124), self-declared cancer free healthy, and colonoscopy screen negative (Table 2). The median age and gender distribution was similar between cases and controls (Table 2).

TABLE 2

| | | Training | | Test | |
|---|---|---|---|---|---|
| Cohort Demographics | | Cohort | | Cohort | |
| Controls: Self- | Total (N) | 155 | | 244 | |
| Declared Cancer | Sex (N, %) | | | | |
| Free & Colonoscopy | Male | 52 | (34%) | 84 | (34%) |
| Screen Negative | Female | 103 | (66%) | 146 | (60%) |
| | Unknown | 0 | (0%) | 14 | (6%) |
| | Median age in | 61 | (38-81) | 63 | (20-85) |
| | years (range) | | | | |
| Colorectal Cancer | Total (N) | 49 | | 124 | |
| Cases | Sex (N) | | | | |
| | Male | 26 | (53%) | 60 | (48%) |
| | Female | 23 | (47%) | 64 | (52%) |
| | Median age in | 66 | (39-80) | 66 | (39-86) |
| | years (range) | | | | |
| | Colorectal Cancer | | | | |
| | Stage (N) | | | | |
| | I/II | 31 | (63%) | 82 | (77%) |
| | III | 13 | (26%) | 28 | (23%) |
| | IV | 5 | (10%) | 0 | (0%) |

There were no significant differences in the median plasma volumes or the cfDNA yield per mL between the training and testing cohorts (Table 3). The assay demonstrated high efficiency in preserving input cfDNA molecules throughout processing, indicating that approximately 80% of all molecules were represented in the sequencing output across the input range. Average molecular diversity per cfDNA input was consistent between samples collected from self-declared cancer free healthy individuals and those collected from the individuals with known CRC (Supplemental FIG. 3). Moreover, affinity-based methylation partitioning was nearly lossless in terms of preserving nearly 90% of all input molecules providing substantial advantages over traditional sodium bisulfite conversion reaction known to lead to loss of 50% of the input DNA.

TABLE 3
Median plasma volume per stage type
for training and testing cohorts.

| | Training | Testing |
|---|---|---|
| No cancer | 7.5 ml | 7.9 ml |
| Stage I | 7.5 ml | 7.9 ml |
| Stage II | 7.8 ml | 7.9 ml |
| Stage III | 7.5 ml | 8.0 ml |

ctDNA Assay Detected 90% of Cancer Samples with 98.6% Specificity

When using pre-specified models and calling threshold to generate binary calls on the testing cohort, the resulting specificity for 74 colonoscopy screen-negative donors was 98.6% (CI: 92.7, 99.9). A lower specificity (91.8%; CI: 86.5, 95.4) was observed when using the same models and calling threshold on a test set of 170 unscreened self-declared cancer free subjects as expected from an estimated 5-10% of the self-reported healthy population having an undiagnosed malignancy The sensitivity of the same model and threshold on 113 early stage cancer patients collected from an independent vendor that was not used for training the model was 90.3% (CI: 83.2, 95; Stage 88.5%; Stage III: 96%).

Epigenomic Estimation of Tumor Fraction Correlates with the Genomic Allelic Frequency

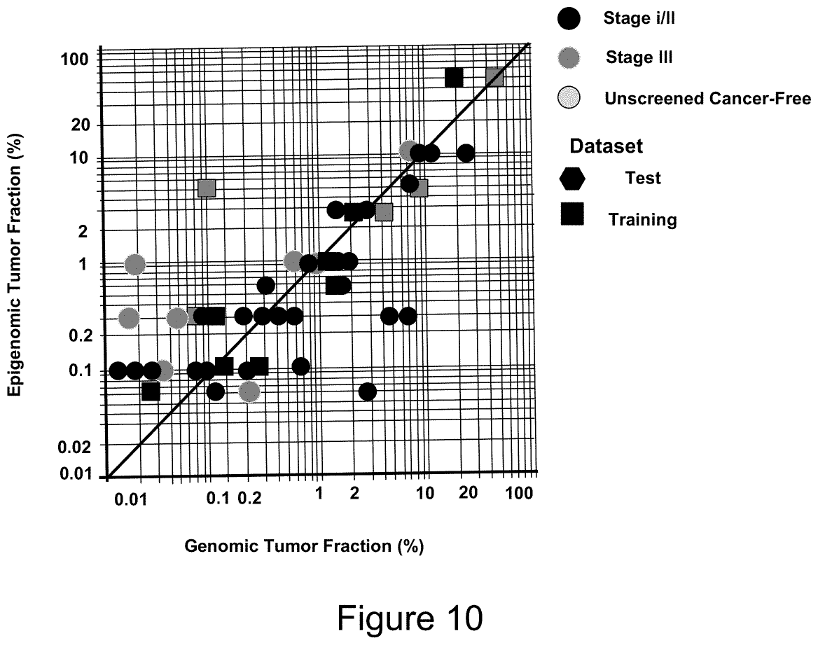

To assess quantitative accuracy of the methylation tumor fraction estimates derived from analysis of methylation partitions, epigenomic tumor fraction estimates were compared to those generated from the maximum allelic frequency associated with somatic call in samples (N=64) that had both positive genomic and epigenomic calls. As can be seen in FIG. 10, the genomic and epigenomic estimates were very well correlated over a long range of tumor fractions. FIG. 10 shows the genomic tumor fraction (x-axis) as estimated by maximal allelic frequency of mutated alleles, compared with epigenomic tumor fraction (y-axis) as estimated by the fraction of ctDNA required in blood to explain the observed epigenomic deviations from expected background. Shown are all Stage I/II, Stage III and cancer free samples that had both positive genomic and epigenomic calls in either the training (squares) or testing set (circles)

Methylation Partition of cfDNA Molecules Aligns with Prior Biological Expectations

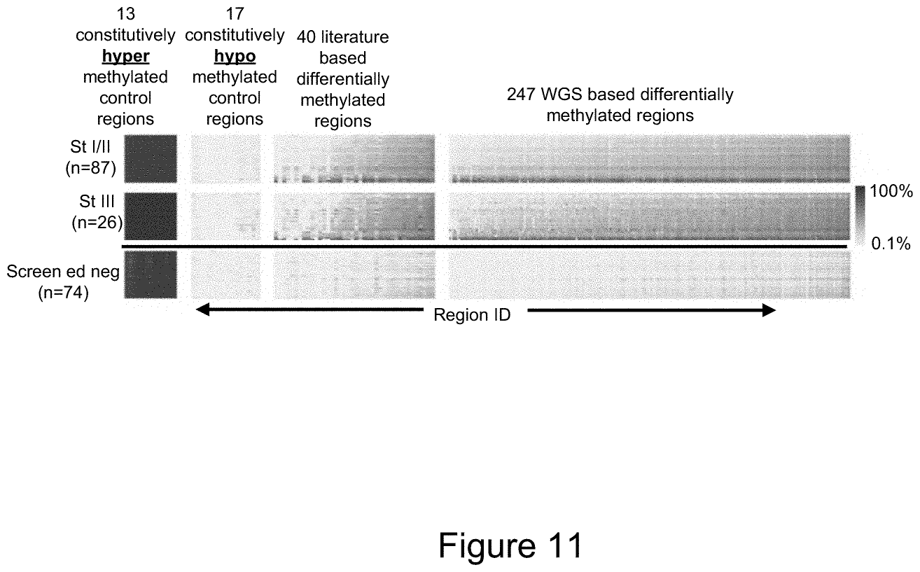

To confirm that the methylation signal corresponds to cfDNA originating from the CRC tumors, the normalized percentage of cfDNA molecules in the hyper-methylated partition per region was visualized (FIG. 11). A strong methylation signal was observed in 40 panel regions selected based on evidence of differential methylation patterns in CRC tumor tissue when compared to a set of control hypo- and hyper-methylated regions (not used in the normalization process), supporting the tumor origin of those fragments in plasma. Looking at a larger set of 247 differentially methylated regions included in the panel based on whole genome sequencing of cfDNA from blood samples healthy donors and late stage CRC patients, a strong and consistent methylation signal that improves differentiation of healthy donors from early stage CRC patients was observed compared to the smaller literature derived set of regions.

The heat map in FIG. 11 shows the normalized fraction of cfDNA reads partitioned to the methylated fraction. The 13 left-most columns show regions known to be ubiquitously hyper-methylated in all tissues, 17 next columns show regions known to be ubiquitously hypo-methylated in all tissues. The next 40 columns showing regions shown in the literature to have differential methylation in CRC tumors. The rightmost 247 regions show additional regions added to the panel based on whole genome sequencing of healthy donors and late stage CRC patients. Each row shows the normalized percent methylation (see methods) in a different test samples. The top 87 rows are Stage I/II CRC patients, next are 26 Stage III patients and bottom 74 rows are screened cancer free donors.

Discussion

ctDNA testing has significant potential to improve cancer care across the spectrum of disease: from screening to advanced cancer treatment selection. However, current methods to improve sensitivity and specificity have failed to generate results that meet clinically relevant performance characteristics and have therefore limited the application of the technology to early stage disease and asymptomatic individuals. Here it is shown a blood based ctDNA assay, utilizing a multi-modal approach to ctDNA detection can achieve clinically significant values for the detection of cancer, with 98.6% specificity and 90.3% sensitivity across Stage I-III CRC.

The affinity-based methylation partitioning implemented in the ctDNA/cancer detection assay (FIG. 9) enables simultaneous assessment of genomic and epigenomic signals using the same input material. Additionally, affinity-based partitioning avoids loss of molecules associated with other epigenomic measurements such as bi-sulfite conversion. Since the molecules of all partitioned bins are eventually sequenced there is very little loss of material (<5% change in molecular diversity). Furthermore, it opens the opportunity to integrate information across analytes in a quantitative way by taking into account the confidence of each independent caller and generating an integrated call.

Overfitting of results was specifically avoided through careful separation of training and testing cohorts. Subsequently, the testing cohort of cancer samples was a completely independent batch of samples from a source unique from the training set. One notable result was the difference in specificities between screened and unscreened age-matched samples. This has a few significant implications. First, by setting the target specificity on screened samples improved sensitivity was observed. It is notable that, specificity estimates differ when evaluated on either screened or unscreened samples. Though some of this difference might arise from cohort variability (as seen in the confidence intervals) some part of it can also be explained by undiagnosed malignancy (CRC) in apparently normal subjects.

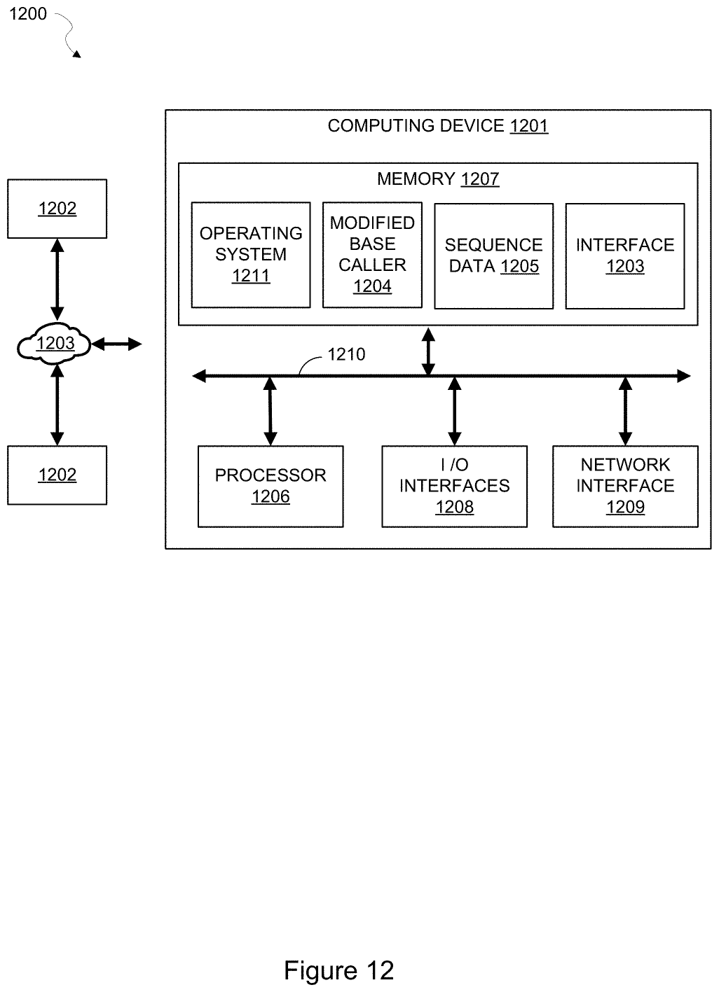

FIG. 12 is a block diagram depicting an environment 1200 comprising non-limiting examples of a computing device 1201 and servers 1202 connected through a network 1203. In an aspect, some or all steps of any described method may be performed on a computing device as described herein. The computing device 1201 can comprise one or multiple computers configured to store one or more of a modified base caller module 1204, sequence data 1205 (e.g., sequence read data, partition information, CG site information, etc.), and the like. The servers 1202 can comprise one or multiple computers configured to store a modified base caller module 1204, sequence data 1205 (e.g., sequence read data, partition information, CG site information, etc.), and the like for remote access. Multiple servers 1202 can communicate with the computing device 1201 via the through the network 1203.

The computing device 1201 and the server 1202 can be a digital computer that, in terms of hardware architecture, generally includes a processor 1206, memory system 1207, input/output (I/O) interfaces 1208, and network interfaces 1209. These components (1206, 1207, 1208, and 1209) are communicatively coupled via a local interface 1210. The local interface 1210 can be, for example, but not limited to, one or more buses or other wired or wireless connections, as is known in the art. The local interface 1210 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 1206 can be a hardware device for executing software, particularly that stored in memory system 1207. The processor 1206 can be any custom made or commercially available processor, a central processing unit (CPU), an auxiliary processor among several processors associated with the computing device 1201 and the server 1202, a semiconductor-based microprocessor (in the form of a microchip or chip set), or generally any device for executing software instructions. When the computing device 1201 and/or the server 1202 is in operation, the processor 1206 can be configured to execute software stored within the memory system 1207, to communicate data to and from the memory system 1207, and to generally control operations of the computing device 1201 and the server 1202 pursuant to the software.

The I/O interfaces 1208 can be used to receive user input from, and/or for providing system output to, one or more devices or components. User input can be provided via, for example, a keyboard and/or a mouse. System output can be provided via a display device and a printer (not shown). I/O interfaces 1208 can include, for example, a serial port, a parallel port, a Small Computer System Interface (SCSI), an infrared (IR) interface, a radio frequency (RF) interface, and/or a universal serial bus (USB) interface.

The network interface 1209 can be used to transmit and receive from the computing device 1201 and/or the server 1202 on the network 1203. The network interface 1209 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 1209 may include address, control, and/or data connections to enable appropriate communications on the network 1203.

The memory system 1207 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 1207 may incorporate electronic, magnetic, optical, and/or other types of storage media. Note that the memory system 1207 can have a distributed architecture, where various components are situated remote from one another, but can be accessed by the processor 1206.

The software in memory system 1207 may include one or more software programs, each of which comprises an ordered listing of executable instructions for implementing logical functions. In the example of FIG. 12, the software in the memory system 1207 of the computing device 1201 can comprise the modified base caller module 1204 (or subcomponents thereof), the sequence data 1205, and a suitable operating system (O/S) 1211. The operating system 1211 essentially controls the execution of other computer programs and provides scheduling, input-output control, file and data management, memory management, and communication control and related services.

For purposes of illustration, application programs and other executable program components such as the operating system 1211 are illustrated herein as discrete blocks, although it is recognized that such programs and components can reside at various times in different storage components of the computing device 1201 and/or the servers 1202. An implementation of the modified base caller module 1204 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

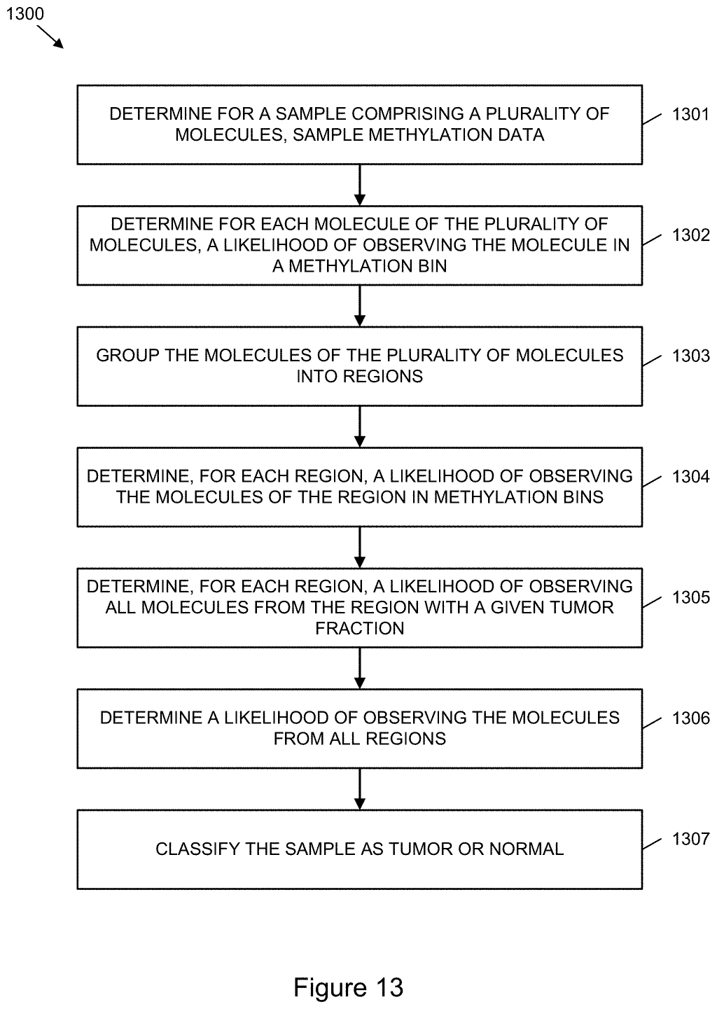

In an implementation, the modified base caller module 1204 may be configured to access the sequence data 1205 and perform a method 1300, shown in FIG. 13. The method 1300 may be performed in whole or in part by a single computing device, a plurality of electronic devices, and the like. The method 1300 may comprise determining, for a sample comprising a plurality of molecules, sample methylation data at step 1301. The sample methylation data may comprise one or more of an indication of a methylation bin of a plurality of methylation bins, a number of CG sites per molecule of the plurality of molecules, a region identifier, combinations thereof, and the like. The sample methylation data may be determined based on one or more FASTQ and/or FASTA files. Determining, for the sample comprising the plurality of molecules, the sample methylation data may comprise receiving and analyzing sequencing data generated by partitioning each molecule of the plurality of molecules into a hypermethylation bin, a residual methylation bin, or a hypomethylation bin, tagging each molecule with a molecular barcode indicating the bin for the molecule, sequencing the molecules to generate sequencing data, and determining, based on the sequencing data, the indication of the methylation bin of the plurality of methylation bins, the number of CG sites per molecule of the plurality of molecules, and the region identifier.

The method 1300 may comprise determining, for each molecule of the plurality of molecules a likelihood P(f|θ, u, v) of observing the molecule in the indicated methylation bin 1302. The determination at 1302 may be based on one or more of MBD binding calibration data, the indication of the methylation bin, the number of CG sites, combinations thereof, and the like. Determining, for each molecule of the plurality of molecules, the likelihood P(f|θ, u, v) of observing the molecule in the indicated methylation bin may comprise determining a tumor methylation rate of tumor derived molecules, determining a normal methylation rate of normal derived molecules, and determining, based on the tumor methylation rate and the normal methylation rate, a portion of tumor derived molecules among all molecules from a region. Determining, for each molecule of the plurality of molecules, the likelihood P(f|θ, u, v) of observing the molecule in the indicated methylation bin may comprise determining a sum of a joint probability that 1) the molecule is tumor derived and the molecule is in a given partition and 2) the probability that the molecule is normal derived and the molecule is in a given partition.

The method 1300 may comprise grouping the molecules of the plurality of molecules into regions at 1303. The grouping at 1303 may be based on the region identifiers.

The method 1300 may comprise determining, for each region, a likelihood P(Dᵢ|θ, u, v) of observing the molecules of the region in the methylation bins of the plurality of methylation bins at 1304. The determination at 1304 may on based on the likelihoods P(f|θ, u, v) of observing the molecules in the indicated methylation bins. For example, the determination at 1304 may comprise multiplying all the likelihoods for a given region.

The method 1300 may comprise determining, for each region, for each possible normal methylation rate and each possible tumor methylation rate a likelihood P(Dᵢ|θ) of observing all molecules from the region with a given tumor fraction at 1305. The determination at 1305 may be based on the likelihoods P(Dᵢ|θ, u, v) of observing the molecules of the region in the methylation bins of the plurality of methylation bins. Determining, for each region, for each possible normal and tumor methylation rates, the likelihood P(Dᵢ|θ) of observing all molecules from the region with a given tumor fraction may comprise defining a prior probability distribution of a normal methylation rate and a tumor methylation rate such that the normal methylation rate is more likely to be low than high while the tumor methylation rate is more likely to be high than low and determining, based on the prior probability distribution, a sum of a product of a probability of observing a given normal methylation rate, a probability of observing a given tumor methylation rate, and a probability of observing all molecules.

The method 1300 may comprise determining, for the sample a likelihood P(D|θ) of observing the molecules from all regions at 1306. The determination at 1306 may be based on the likelihoods P(Dᵢ|θ) of observing all molecules from the regions with the given tumor fractions. Determining, for the sample, the likelihood P(D|θ) of observing the molecules from all regions may comprise determining a product of the probability of observing all molecules from each region.

The method 1300 may comprise classifying the sample as tumor or normal at 1307. The determination at 1307 may be based on the likelihoods P(D|θ) of observing the molecules from all regions.

Classifying the sample as tumor or normal may comprise determining, based on the largest likelihood P(D|θ) of observing all molecules from the region with the given tumor fraction, a tumor fraction for the sample and classifying, based on the tumor fraction for the sample, the sample as tumor or normal. Classifying, based on the tumor fraction for the sample, the sample as tumor or normal comprises determining, based on the tumor fraction for the sample exceeding a threshold, the sample as tumor.

Classifying the sample as tumor or normal may comprise determining, based on the likelihoods P(D|θ) of observing the molecules from all regions, a posterior mean tumor fraction and classifying, based on the posterior mean tumor fraction for the sample, the sample as tumor or normal. Classifying, based on the tumor fraction for the sample, the sample as tumor or normal may comprise determining, based on the posterior mean tumor fraction for the sample exceeding a threshold, the sample as tumor.

The method 1300 may further comprise determining the MBD binding calibration data.

Determining the MBD binding calibration data may comprise determining, for each methylated control molecule of a plurality of methylated control molecules, an indication of a methylation bin of the plurality of methylation bins and a number of CG sites per methylated control molecule of the plurality of methylated control molecules, plotting based on the indication of the methylation bin and the number of CG sites of each methylated control molecule, a hypermethylation binding curve, determining, for each unmethylated control molecule of a plurality of unmethylated control molecules, an indication of a methylation bin of the plurality of methylation bins and a number of CG sites per unmethylated control molecule of the plurality of unmethylated control molecules, and plotting based on the indication of the methylation bin and the number of CG sites of each unmethylated control molecule, a hypomethylation binding curve.

Determining the MBD binding calibration data may comprise determining, based on a first wash, for each control molecule of a plurality of control molecules, a first indication that the control molecule is bound to MBD or a second indication that the control molecule is not bound to MBD, estimating, for the plurality of control molecules, based on the first indications and the second indications, a first binding energy (e_{bound}) and a second binding energy (e_{unbound}), determining, based on a given number of methylated CG sites, the first binding energy, and the second binding energy, a likelihood (P(bound)) of a molecule to bind to MBD after the first wash and a likelihood (P(unbound)) of a molecule to not bind to MBD after the first wash, plotting based on the given numbers of methylated CG sites and the likelihood (P(unbound)) of a molecule to not bind to MBD after the first wash, a hypomethylation binding curve, determining, based on a second wash, for each control molecule of the plurality of control molecules having an indication that the control molecule is bound to MBD in the first wash, a third indication that the control molecule is bound to MBD or a fourth indication that the control molecule is not bound to MBD, estimating, for the plurality of control molecules having an indication that the control molecule is bound to MBD in the first wash, based on the third indications and the fourth indications, a third binding energy (e_{hyper}) and a fourth binding energy (e_{hypo}), determining, based on a given number of methylated CG sites, the third binding energy, and the fourth binding energy, a likelihood (P(hyper)) of a molecule to bind to MBD after the second wash and a likelihood (P(residual)) of a molecule to not bind to MBD after the second wash, and plotting based on the given numbers of methylated CG sites and the likelihood (P(hyper)) of a molecule to bind to MBD after the second wash, a hypermethylation binding curve.

Determining the MBD binding calibration data may comprise determining a likelihood of a molecule to bind to MBD after a first wash, determining a likelihood of a molecule to not bind to MBD after the first wash, determining, based on the likelihood of the molecule to bind to MBD after the first wash, a likelihood that the molecule that bound to MBD after the first wash binds to MBD after a second wash, determining, based on the likelihood of the molecule to bind to MBD after the first wash, a likelihood that the molecule that bound to MBD after the first wash does not bind to MBD after the second wash, plotting, based on a given number of methylated CG sites and the likelihood of a molecule to not bind to MBD after the first wash, a hypomethylation binding curve, and plotting, based on the given number of methylated CG sites and the likelihood that the molecule that bound to MBD after the first wash binds to MBD after the second wash, a hypermethylation binding curve.

Determining the MBD binding calibration data may comprise aggregating, for a plurality of samples, unmethylated CG site count data for a plurality of molecules and associated likelihoods of observing a molecule in a methylation bin of the plurality of methylation bins and determining, based on the unmethylated CG site count data and the associated likelihoods of observing a molecule in a methylation bin of the plurality of methylation bins, a negative MBD binding curve associated with each of a hypermethylation bin, a residual methylation bin, and a hypomethylation bin.

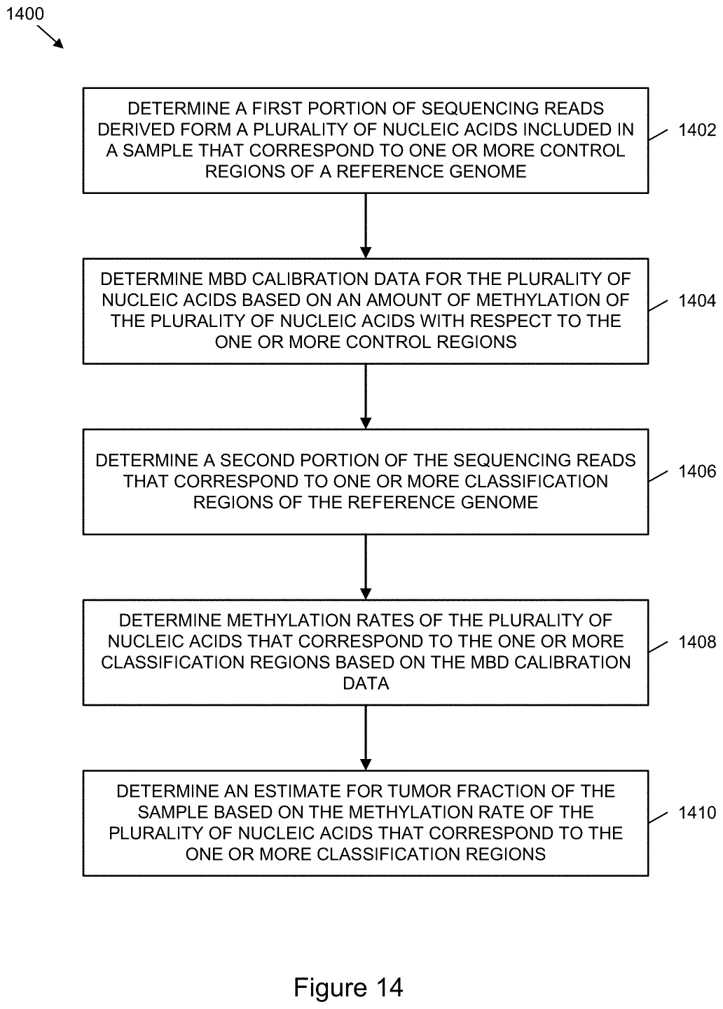

FIG. 14 illustrates a flow diagram of a process 1400 to determine an estimate for tumor fraction of a sample based on calibration data and partitioning of nucleic acids according to binding with methyl binding domain (MBD). The process 1400 can also be performed by the system 1200. The process 1400 includes, at operation 1402, determining a first portion of sequence reads derived from a plurality of nucleic acids included in a sample that correspond to one or more control regions of a reference genome. In one or more examples, the sample includes a first number of nucleic acids derived from blood or tissue of a subject and a second number of synthetic nucleic acids that include nucleotide sequences that correspond to the one or more control regions. In various examples, the second number of synthetic nucleic acids can include spike-in control nucleic acids that are added to the first number of nucleic acids after the first number of nucleic acids are extracted from the blood or tissue of the subject.

The sequence reads can be included in sequencing data that is generated by a sequencing apparatus performing one or more sequencing operations with respect to the sample. In one or more examples, the sequencing data can include millions up to billions of sequencing reads. Individual sequencing reads can indicate a nucleotide sequence of nucleic acids included in the sample. In various examples, individual sequencing reads can also indicate an MBD binding partition of a plurality of MBD binding partitions. Individual MBD binding partitions can correspond to an amount of methylation of cytosine-guanine (CG) regions of the nucleotide sequence. CG regions of the sequencing reads can include at least a threshold number of CG pairs. Individual CG pairs can correspond to a cytosine nucleotide following by a guanine nucleotide in a nucleotide sequence. The threshold number of CG pairs can be at least one, at least three, at least five, at least eight, at least ten, at least fifteen, or at least twenty.

The amount of methylation of the CG regions can correspond to a strength of binding to MBD. In one or more illustrative examples, a strength of binding to MBD can correspond to a binding energy between a nucleic acid and MBD. In one or more examples, the strength of binding of MBD can correspond to a concentration of sodium chloride (NaCl) in a solution that contacts a nucleic acid bound to MBD. The strength of a nucleic acid binding to MBD can also correspond to a number of methylated CG regions of the nucleic acid. In one or more examples, solutions with increasing salt concentration can separate nucleic acids having increased binding strength to MBD.

In various examples, a first partition of the plurality of partitions can correspond to a first range of binding strengths of nucleic acids to MBD and to a first range of methylated CG regions and a second partition of the plurality of partitions can correspond to a second range of binding strengths of nucleic acids to MBD and to a second range of methylated CG regions. The first range of binding strengths can be less than the second range of binding strengths. In one or more scenarios, a first solution having a first NaCl concentration can separate a first group of nucleic acids having the first range of binding strengths from MBD and a second solution having a second NaCl concentration can separate a second group of nucleic acids having the second range of binding strengths from MBD with the second NaCl concentration being greater than the first NaCl concentration. Additionally, a third partition of the plurality of partitions can correspond to a third range of binding strengths and a third range of methylated CG regions. The third range of binding strengths can be greater than the first range of binding strengths and the second range of binding strengths. In one or more instances, a third solution having a third NaCl concentration can separate a third group of nucleic acids having the third range of binding strengths from NaCl. The third NaCl concentration can be greater than the first NaCl concentration and the second NaCl concentration.

In one or more illustrative examples, a plurality of nucleic acids derived from at least one of blood or tissue of a subject can be combined with a solution including an amount of MBD to produce a nucleic acid-MBD solution. A first wash of the nucleic acid-MBD solution can be performed with a first solution including a first NaCl concentration to produce a first nucleic acid fraction and a first residual solution. The first nucleic acid fraction can include a first portion of the plurality of nucleic acids and the first residual solution can include a second portion of the plurality of nucleic acids. In one or more examples, the first portion of the plurality of nucleic acids can have a first range of binding energies to MBD that are less than a second range of binding energies to MBD of the second portion of the plurality of nucleic acids.

Additionally, a second wash of the first residual solution can be performed with a second solution including a second concentration of NaCl that is greater than the first concentration of NaCl to produce a second nucleic acid fraction and a second residual solution. The second nucleic acid fraction can include a first subset of the second portion of the plurality of nucleic acids and the second residual solution can include a second subset of the second portion of the plurality of nucleic acids. The first subset of the second portion of the plurality of nucleic acids can have a third range of binding energies to MBD that are less than a fourth range of binding energies to MBD of the second subset of the second portion of the plurality of nucleic acids. Further, a third wash of the second residual solution can be performed with a third solution including a third concentration of NaCl that is greater than the second concentration of NaCl to produce a third nucleic acid fraction that includes the second subset of the second portion of the plurality of nucleic acids.

Subsequent to the first wash, the second wash, and the third wash a determination can be made that the first portion of the plurality of nucleic acids are associated with the first partition of the plurality of partitions. A first molecular barcode can then be attached to the first portion of the plurality of nucleic acids with the first molecular barcode indicating the first partition. In this way, a sequencing read that corresponds to the first partition can be identified based on determining that the sequencing read includes the first molecular barcode. In addition, a determination can be made that the first subset of the second portion of the plurality of nucleic acids is associated with an additional partition of the plurality of partitions. In these situations, a second molecular barcode can be attached to the second portion of the plurality of nucleic acids with the second molecular barcode indicating the additional partition. As a result, a sequencing read that corresponds to the additional partition can be identified based on determining that the sequencing read includes the second molecular barcode. Further, a determination can be made that the second subset of the second portion of the plurality of nucleic acids is associated with the second partition. A third molecular barcode can then be attached to the second subset of the second portion of the plurality of nucleic acids with the third molecular barcode indicating the second partition. In these instances, a sequencing read that corresponds to the second partition can be identified based on determining that the sequencing read includes the third molecular barcode.

In various examples, the one or more control regions can be determined by performing an alignment process to determine amounts of homology between at least a portion of the sequencing reads included in the sequencing data and one or more nucleotide sequences of a reference genome that correspond to the one or more control regions. In one or more examples, a subset of the sequencing reads that corresponds to the one or more control regions can be determined by determining a number of the sequencing reads having at least a threshold amount of homology with at least one control region of the one or more control regions.

In addition, at operation 1404, the process 1400 includes determining MBD calibration data for the plurality of nucleic acids based on an amount of methylation of the plurality of nucleic acids with respect to the one or more control regions. The MBD calibration data can be determined based on an amount of methylation of control regions of the sequencing reads included in the sequencing data. In various examples, the sequencing data can be analyzed to determine a first subset of sequencing reads that correspond to a first partition of the plurality of partitions. The first subset of sequencing reads can correspond to a first portion of the MBD calibration data that can be determined using sequencing reads having at least first threshold number of methylated CG regions. The first portion of the MBD calibration data can correspond to positive control MBD calibration data. Additionally, a second portion of the MBD calibration date can be determined using sequencing reads having no greater than a second threshold number of methylated regions. The second portion of the MBD calibration data can correspond to negative control MBD data.

With respect to the first portion of the MBD calibration data, a first number of sequencing reads of the first subset of sequencing reads can correspond to a first partition of the plurality of partitions. The first partition can correspond to a first range of numbers of methylated CG regions. In one or more examples, the first subset of sequencing reads can have a number of methylated CG regions within the first range of numbers of methylated CG regions. Additionally, a second number of sequencing reads of the first subset of sequencing reads can correspond to a second partition of the plurality of partitions. The second partition can correspond to a second range of numbers of methylated CG regions that is different from the first range of numbers of methylated CG regions. In various examples, the second subset of sequencing reads can have a number of methylated CG regions within the second range of numbers of methylated CG regions.

In one or more examples, the MBD calibration data is generated using the first number of sequencing reads and the second number of sequencing reads such that the MBD calibration data indicates a first number of probabilities of additional nucleic acids included in the sample being associated with the first partition. Additionally, the MBD calibration data can indicate a second number of probabilities of the additional nucleic acids included in the sample being associated with a second partition. In various examples, the additional nucleic acids can include nucleic acids included in the sample that do not include a control region of the one or more control region.

The negative control MBD calibration data can be determined by analyzing the sequencing data to determine an additional subset of the sequencing reads that include one or more additional control regions having no greater than an additional threshold number of methylated CG regions with the additional threshold number of methylated CG regions being less than the threshold number of methylated CG regions for the positive control MBD calibration data. In this way, the negative control MBD calibration data has a lower rate of methylation of CG regions than the positive control MBD calibration data. Partitions that correspond to the additional subset of sequencing reads of the negative control MBD calibration data can be determined. For example, a number of sequencing reads of the additional subset of sequencing reads can be determined that correspond to the first partition of the plurality of partitions and another group of the sequencing reads of the additional subset of sequencing reads can be determined that correspond to the second partition of the plurality of partitions. Further, the negative control MBD calibration data can indicate an additional number of probabilities of the additional nucleic acids being associated with the first partition and a further number of probabilities of the additional nucleic acids being associated with the second partition.

The process 1400 also includes, at operation 1406, determining a second portion of the sequencing reads that correspond to one or more classification regions of the reference genome. The one or more classification regions can include at least one of a first CG region that has at least a first threshold amount of methylation in cells derived from a tumor and a second CG region that has no greater a second threshold amount of methylation in non-tumor derived cells. The first threshold amount of methylation being greater than the second threshold amount of methylation. In this way, the classification regions can include genomic regions that have CG regions with relatively high amounts of methylation in tumor cells and genomic regions that have CG regions with relatively low amounts of methylation in non-tumor cells.

In various examples, the one or more classification regions can be determined by performing an additional alignment process to determine amounts of homology between at least a portion of the sequencing reads included in the sequencing data and one or more nucleotide sequences of a reference genome that correspond to the one or more classification regions. In one or more examples, an additional subset of the sequencing reads that corresponds to the one or more classification regions can be determined by determining, based on the alignment process, the second subset of sequencing reads by determining a number of the sequencing reads having at least a threshold amount of homology with at least one classification region of the one or more classification regions. In one or more illustrative examples, the one or more classification regions correspond to individual probes of an assay that indicate genomic regions related to a presence of a tumor.

Further, at operation 1408, the process 1400 includes determining methylation rates of the plurality of nucleic acids that correspond to the one or more classification regions based on the MBD calibration data. The methylation rates can correspond to respective amounts of methylation of the one or more classification regions. In various examples, determining the methylation rates of nucleic acids that correspond to the one or more classification regions can include determining partition data for the subset of sequencing reads that correspond to the one or more classification regions. In one or more examples, the partition data can indicate a partition of the plurality of partitions that correspond to respective amounts of methylation of CG regions included in the one or more classification regions. The partitions can be indicated in the sequencing reads by partition tags that are molecular barcodes that correspond to the respective partitions.

Additionally, methylation rates of CG regions of the sequencing reads that correspond to the one or more classification regions can also be determined based on a number of CG regions of the nucleic acids corresponding to the sequencing reads. The number of CG regions of nucleic acids can be determining by analyzing the sequencing reads and identifying portions of the sequencing reads that include at least a threshold number of CG pairs. In various examples, the number of CG pairs can be consecutively locating in the nucleic acid sequences. In one or more examples, the amount of methylation of classification regions can be determined by determining a respective likelihood for a plurality of candidate amounts of methylation of the number of CG regions in the individual classification regions of the one or more classification regions based on the analysis of the partition data and the CG region data in relation to the MBD calibration data. In one or more illustrative examples, the likelihood can be determined for a number of individual candidate amounts of methylation until a maximum respective likelihood is determined for a particular candidate amount of methylation of a plurality of candidate amounts. In one or more scenarios, the maximum likelihood for candidate amounts of methylation can be determined for a number of candidate amounts of methylation for each classification region and can result in hundreds of calculations up to thousands of calculations or more to determine respective likelihoods in order to identify a candidate amount of methylation for the one or more classification regions that corresponds to the maximum likelihood.

At operation, 1410, the process 1400 includes determining an estimate for tumor fraction of the sample based on the methylation rate of the plurality of nucleic acids that correspond to the one or more classification regions. In one or more examples, the estimate for the tumor fraction can be determined by maximizing a likelihood of a number of candidate estimates for the tumor fraction of the sample. In various examples, hundreds, up to thousands of calculations for candidate estimates for tumor fraction can be performed to maximize the likelihood of the estimate for the tumor fraction with respect to the partition data and the CG region data for the sample in relation to the MBD calibration data across a number of calibration regions. In various examples, the estimate for the tumor fraction of the sample can be used to determine a probability of a presence of a tumor in the subject from which the sample is derived based on the tumor fraction. In one or more illustrative examples, as the estimate for the tumor fraction of samples increases, the probability of the presence of a tumor in the subject from which the sample is derived can also increase.

In various examples, the estimate for the tumor fraction of the sample can be determined based on methylation rates of CG regions of the one or more classification regions in relation to one or more methylation rates of tumor cells and methylation rates of CG regions of the one or more classification regions in relation to one or more methylation rates of non-tumor cells. In one or more examples, the methylation rates of the one or more classification regions of non-tumor cells can be less than the methylation rates of the one or more classification regions in tumor cells. Candidate estimates for the tumor fraction of the sample can be determined with respect to the different methylation rates related to tumor cells and non-tumor cells. In one or more illustrative examples, the estimate for the tumor fraction can be determined by determining a first likelihood of a candidate estimate of the tumor fraction for the sample based on a plurality of first methylation rates of nucleic acids derived from the sample that correspond to a classification region of the one or more classification regions where the plurality of first methylation rates corresponding to the methylation rates in tumor cells. Additionally, the estimate for the tumor fraction can be determined by determining a second likelihood of the candidate estimate of the tumor fraction for the sample based on a plurality of second methylation rates of the nucleic acids derived from the sample that correspond to the classification region of the one or more classification regions where the plurality of second methylation rates corresponding to methylation rates in non-tumor cells.

A numbered non-limiting list of aspects of the present subject matter is presented below.

Aspect 1. A method comprising: determining, for a sample comprising a plurality of molecules, sample methylation data comprising an indication of a methylation bin of a plurality of methylation bins, a number of CG sites per molecule of the plurality of molecules, and a region identifier; determining, for each molecule of the plurality of molecules, based on: MBD binding calibration data, the indication of the methylation bin, and the number of CG sites, a likelihood P(f|θ,u,v) of observing the molecule in the indicated methylation bin; grouping, based on the region identifiers, the molecules of the plurality of molecules into regions; determining, for each region, based on the likelihoods P(f|θ,u,v) of observing the molecules in the indicated methylation bins, a likelihood P(Dᵢ|θ, u, v) of observing the molecules of the region in the methylation bins of the plurality of methylation bins; determining, for each region, for each possible normal methylation rate and each possible tumor methylation rate, based on the likelihoods P(Dᵢ|θ, u,v) of observing the molecules of the region in the methylation bins of the plurality of methylation bins, a likelihood P(Dᵢ|θ) of observing all molecules from the region with a given tumor fraction; determining, for the sample, based on the likelihoods P(Dᵢ|θ) of observing all molecules from the regions with the given tumor fractions, a likelihood P(D|θ) of observing the molecules from all regions; and classifying, based on the likelihoods P(D|θ) of observing the molecules from all regions, the sample as tumor or normal.

Aspect 2. The method of aspect 1, wherein determining, for the sample comprising the plurality of molecules, the sample methylation data comprises: partitioning each molecule of the plurality of molecules into a hypermethylation bin, a residual methylation bin, or a hypomethylation bin; tagging each molecule with a molecular barcode indicating the bin for the molecule; sequencing the molecules to generate sequencing data; and determining, based on the sequencing data, the indication of the methylation bin of the plurality of methylation bins, the number of CG sites per molecule of the plurality of molecules, and the region identifier.

Aspect 3. The method of aspect 1 or 2, wherein determining, for each molecule of the plurality of molecules, the likelihood P(f|θ, u, v) of observing the molecule in the indicated methylation bin comprises: determining a tumor methylation rate of tumor derived molecules; determining a normal methylation rate of normal derived molecules; and determining, based on the tumor methylation rate and the normal methylation rate, a portion of tumor derived molecules among all molecules from a region.

Aspect 4. The method of any one of aspects 1-3, wherein determining, for each molecule of the plurality of molecules, the likelihood P(f|θ, u, v) of observing the molecule in the indicated methylation bin comprises determining a sum of a joint probability that 1) the molecule is tumor derived and the molecule is in a given partition and 2) the probability that the molecule is normal derived and the molecule is in a given partition.

Aspect 5. The method of any one of aspects 1-4, wherein determining, for each region, for each possible normal and tumor methylation rates, the likelihood P(Dᵢ|θ) of observing all molecules from the region with a given tumor fraction comprises: defining a prior probability distribution of a normal methylation rate and a tumor methylation rate such that the normal methylation rate is more likely to be low than high while the tumor methylation rate is more likely to be high than low; and determining, based on the prior probability distribution, a sum of a product of: a probability of observing a given normal methylation rate, a probability of observing a given tumor methylation rate, and a probability of observing all molecules.

Aspect 6. The method of any one of aspects 1-5, wherein determining, for the sample, the likelihood P(D|θ) of observing the molecules from all regions comprises determining a product of the probability of observing all molecules from each region.

Aspect 7. The method of any one of aspects 1-6, further comprising determining the MBD binding calibration data.

Aspect 8. The method of aspect 7, wherein determining the MBD binding calibration data comprises: determining, for each methylated control molecule of a plurality of methylated control molecules, an indication of a methylation bin of the plurality of methylation bins and a number of CG sites per methylated control molecule of the plurality of methylated control molecules; plotting based on the indication of the methylation bin and the number of CG sites of each methylated control molecule, a hypermethylation binding curve; determining, for each unmethylated control molecule of a plurality of unmethylated control molecules, an indication of a methylation bin of the plurality of methylation bins and a number of CG sites per unmethylated control molecule of the plurality of unmethylated control molecules; and plotting based on the indication of the methylation bin and the number of CG sites of each unmethylated control molecule, a hypomethylation binding curve.

Aspect 9. The method of aspect 7, wherein determining the MBD binding calibration data comprises: determining, based on a first wash, for each control molecule of a plurality of control molecules, a first indication that the control molecule is bound to MBD or a second indication that the control molecule is not bound to MBD; estimating, for the plurality of control molecules, based on the first indications and the second indications, a first binding energy and a second binding energy; determining, based on a given number of methylated CG sites, the first binding energy, and the second binding energy, a likelihood (P(bound)) of a molecule to bind to MBD after the first wash and a likelihood (P(unbound)) of a molecule to not bind to MBD after the first wash; plotting based on the given numbers of methylated CG sites and the likelihood (P(unbound)) of a molecule to not bind to MBD after the first wash, a hypomethylation binding curve; determining, based on a second wash, for each control molecule of the plurality of control molecules having an indication that the control molecule is bound to MBD in the first wash, a third indication that the control molecule is bound to MBD or a fourth indication that the control molecule is not bound to MBD; estimating, for the plurality of control molecules having an indication that the control molecule is bound to MBD in the first wash, based on the third indications and the fourth indications, a third binding energy and a fourth binding energy; determining, based on a given number of methylated CG sites, the third binding energy, and the fourth binding energy, a likelihood (P(hyper)) of a molecule to bind to MBD after the second wash and a likelihood (P(residual)) of a molecule to not bind to MBD after the second wash; and plotting based on the given numbers of methylated CG sites and the likelihood (P(hyper)) of a molecule to bind to MBD after the second wash, a hypermethylation binding curve.

Aspect 10. The method of aspect 7, wherein determining the MBD binding calibration data comprises: determining a likelihood of a molecule to bind to MBD after a first wash; determining a likelihood of a molecule to not bind to MBD after the first wash; determining, based on the likelihood of the molecule to bind to MBD after the first wash, a likelihood that the molecule that bound to MBD after the first wash binds to MBD after a second wash; determining, based on the likelihood of the molecule to bind to MBD after the first wash, a likelihood that the molecule that bound to MBD after the first wash does not bind to MBD after the second wash; plotting, based on a given number of methylated CG sites and the likelihood of a molecule to not bind to MBD after the first wash, a hypomethylation binding curve; and plotting, based on the given number of methylated CG sites and the likelihood that the molecule that bound to MBD after the first wash binds to MBD after the second wash, a hypermethylation binding curve.

Aspect 11. The method of aspect 7, wherein determining the MBD binding calibration data comprises: aggregating, for a plurality of samples, unmethylated CG site count data for a plurality of molecules and associated likelihoods of observing a molecule in a methylation bin of the plurality of methylation bins; and determining, based on the unmethylated CG site count data and the associated likelihoods of observing a molecule in a methylation bin of the plurality of methylation bins, a negative MBD binding curve associated with each of a hypermethylation bin, a residual methylation bin, and a hypomethylation bin.

Aspect 12. The method of any one of aspects 1-11, wherein classifying, based on the likelihood P(D|θ) of observing the molecules from all regions, the sample as tumor or normal comprises: determining, based on the largest likelihood P(D|θ) of observing all molecules from the region with the given tumor fraction, a tumor fraction for the sample; and classifying, based on the tumor fraction for the sample, the sample as tumor or normal.

Aspect 13. The method of aspect 12, wherein classifying, based on the tumor fraction for the sample, the sample as tumor or normal comprises determining, based on the tumor fraction for the sample exceeding a threshold, the sample as tumor.

Aspect 14. The method of any one of aspects 1-13, wherein classifying, based on the likelihood P(D|θ) of observing the molecules from all regions, the sample as tumor or normal comprises: determining, based on the likelihoods P(D|θ) of observing the molecules from all regions, a posterior mean tumor fraction; and classifying, based on the posterior mean tumor fraction for the sample, the sample as tumor or normal.

Aspect 15. The method of aspect 14, wherein classifying, based on the tumor fraction for the sample, the sample as tumor or normal comprises determining, based on the posterior mean tumor fraction for the sample exceeding a threshold, the sample as tumor.

Aspect 16. An apparatus comprising: one or more processors; and memory storing processor executable instructions that, when executed by the one or more processors, cause the apparatus to: determine, for a sample comprising a plurality of molecules, sample methylation data comprising an indication of a methylation bin of a plurality of methylation bins, a number of CG sites per molecule of the plurality of molecules, and a region identifier; determine, for each molecule of the plurality of molecules, based on: MBD binding calibration data, the indication of the methylation bin, and the number of CG sites, a likelihood P(f|θ, u, v) of observing the molecule in the indicated methylation bin; group, based on the region identifiers, the molecules of the plurality of molecules into regions; determine, for each region, based on the likelihoods P(f|θ,u,v) of observing the molecules in the indicated methylation bins, a likelihood P(Dᵢ|θ, u, v) of observing the molecules of the region in the methylation bins of the plurality of methylation bins; determine, for each region, for each possible normal methylation rate and each possible tumor methylation rate, based on the likelihoods P(Dᵢ|θ, u, v) of observing the molecules of the region in the methylation bins of the plurality of methylation bins, a likelihood P(Dᵢ|θ) of observing all molecules from the region with a given tumor fraction; determine, for the sample, based on the likelihoods P(Dᵢ|θ) of observing all molecules from the regions with the given tumor fractions, a likelihood P(D|θ) of observing the molecules from all regions; and classify, based on the likelihoods P(D|θ) of observing the molecules from all regions, the sample as tumor or normal.

Aspect 17. The apparatus of aspect 16, wherein the processor executable instructions that, when executed by the one or more processors, cause the apparatus to determine, for the sample comprising the plurality of molecules, the sample methylation data further cause the apparatus to:

partition each molecule of the plurality of molecules into a hypermethylation bin, a residual methylation bin, or a hypomethylation bin; tag each molecule with a molecular barcode indicating the bin for the molecule; sequence the molecules to generate sequencing data; and determine, based on the sequencing data, the indication of the methylation bin of the plurality of methylation bins, the number of CG sites per molecule of the plurality of molecules, and the region identifier.

Aspect 18. The apparatus of aspect 16 or 17, wherein the processor executable instructions that, when executed by the one or more processors, cause the apparatus to determine, for each molecule of the plurality of molecules, the likelihood P(f|θ,u,v) of observing the molecule in the indicated methylation bin further cause the apparatus to: determine a tumor methylation rate of tumor derived molecules; determine a normal methylation rate of normal derived molecules; and determine, based on the tumor methylation rate and the normal methylation rate, a portion of tumor derived molecules among all molecules from a region.

Aspect 19. The apparatus of any one of aspects 16-18, wherein the processor executable instructions that, when executed by the one or more processors, cause the apparatus to determine, for each molecule of the plurality of molecules, the likelihood P(f|θ, u, v) of observing the molecule in the indicated methylation bin further cause the apparatus to determine a sum of a joint probability that 1) the molecule is tumor derived and the molecule is in a given partition and 2) the probability that the molecule is normal derived and the molecule is in a given partition.

Aspect 20. The apparatus of any one of aspects 16-19, wherein the processor executable instructions that, when executed by the one or more processors, cause the apparatus to determine, for each region, for each possible normal and tumor methylation rates, the likelihood P(Dᵢ|θ) of observing all molecules from the region with a given tumor fraction further cause the apparatus to: define a prior probability distribution of a normal methylation rate and a tumor methylation rate such that the normal methylation rate is more likely to be low than high while the tumor methylation rate is more likely to be high than low; and determine, based on the prior probability distribution, a sum of a product of: a probability of observing a given normal methylation rate, a probability of observing a given tumor methylation rate, and a probability of observing all molecules.

Aspect 21. The apparatus of any one of aspects 16-20, wherein the processor executable instructions that, when executed by the one or more processors, cause the apparatus to determine, for the sample, the likelihood P(D|θ) of observing the molecules from all regions further cause the apparatus to determine a product of the probability of observing all molecules from each region.

Aspect 22. The apparatus of any one of aspects 16-21, wherein the processor executable instructions that, when executed by the one or more processors, further cause the apparatus to determine the MBD binding calibration data.

Aspect 23. The apparatus of aspect 22, wherein the processor executable instructions that, when executed by the one or more processors, cause the apparatus to determine the MBD binding calibration data further cause the apparatus to: determine, for each methylated control molecule of a plurality of methylated control molecules, an indication of a methylation bin of the plurality of methylation bins and a number of CG sites per methylated control molecule of the plurality of methylated control molecules; plot based on the indication of the methylation bin and the number of CG sites of each methylated control molecule, a hypermethylation binding curve; determine, for each unmethylated control molecule of a plurality of unmethylated control molecules, an indication of a methylation bin of the plurality of methylation bins and a number of CG sites per unmethylated control molecule of the plurality of unmethylated control molecules; and plot based on the indication of the methylation bin and the number of CG sites of each unmethylated control molecule, a hypomethylation binding curve.

Aspect 24. The apparatus of aspect 22, wherein the processor executable instructions that, when executed by the one or more processors, cause the apparatus to determine the MBD binding calibration data further cause the apparatus to: determine, based on a first wash, for each control molecule of a plurality of control molecules, a first indication that the control molecule is bound to MBD or a second indication that the control molecule is not bound to MBD; estimate, for the plurality of control molecules, based on the first indications and the second indications, a first binding energy and a second binding energy; determine, based on a given number of methylated CG sites, the first binding energy, and the second binding energy, a likelihood (P(bound)) of a molecule to bind to MBD after the first wash and a likelihood (P(unbound)) of a molecule to not bind to MBD after the first wash; plot based on the given numbers of methylated CG sites and the likelihood (P(unbound)) of a molecule to not bind to MBD after the first wash, a hypomethylation binding curve; determine, based on a second wash, for each control molecule of the plurality of control molecules having an indication that the control molecule is bound to MBD in the first wash, a third indication that the control molecule is bound to MBD or a fourth indication that the control molecule is not bound to MBD; estimate, for the plurality of control molecules having an indication that the control molecule is bound to MBD in the first wash, based on the third indications and the fourth indications, a third binding energy and a fourth binding energy; determine, based on a given number of methylated CG sites, the third binding energy, and the fourth binding energy, a likelihood (P(hyper)) of a molecule to bind to MBD after the second wash and a likelihood (P(residual)) of a molecule to not bind to MBD after the second wash; and plot based on the given numbers of methylated CG sites and the likelihood (P(hyper)) of a molecule to bind to MBD after the second wash, a hypermethylation binding curve.

Aspect 25. The apparatus of aspect 22, wherein the processor executable instructions that, when executed by the one or more processors, cause the apparatus to determine the MBD binding calibration data further cause the apparatus to: determine a likelihood of a molecule to bind to MBD after a first wash; determine a likelihood of a molecule to not bind to MBD after the first wash; determine, based on the likelihood of the molecule to bind to MBD after the first wash, a likelihood that the molecule that bound to MBD after the first wash binds to MBD after a second wash; determine, based on the likelihood of the molecule to bind to MBD after the first wash, a likelihood that the molecule that bound to MBD after the first wash does not bind to MBD after the second wash; plot, based on a given number of methylated CG sites and the likelihood of a molecule to not bind to MBD after the first wash, a hypomethylation binding curve; and plot, based on the given number of methylated CG sites and the likelihood that the molecule that bound to MBD after the first wash binds to MBD after the second wash, a hypermethylation binding curve.

Aspect 26. The apparatus of aspect 22, wherein the processor executable instructions that, when executed by the one or more processors, cause the apparatus to determine the MBD binding calibration data further cause the apparatus to: aggregate, for a plurality of samples, unmethylated CG site count data for a plurality of molecules and associated likelihoods of observing a molecule in a methylation bin of the plurality of methylation bins; and determine, based on the unmethylated CG site count data and the associated likelihoods of observing a molecule in a methylation bin of the plurality of methylation bins, a negative MBD binding curve associated with each of a hypermethylation bin, a residual methylation bin, and a hypomethylation bin.

Aspect 27. The apparatus of any one of aspects 16-26, wherein the processor executable instructions that, when executed by the one or more processors, cause the apparatus to classify, based on the likelihood P(D|θ) of observing the molecules from all regions, the sample as tumor or normal further cause the apparatus to: determine, based on the largest likelihood P(D|θ) of observing all molecules from the region with the given tumor fraction, a tumor fraction for the sample; and classify, based on the tumor fraction for the sample, the sample as tumor or normal.

Aspect 28. The apparatus of aspect 27, wherein the processor executable instructions that, when executed by the one or more processors, cause the apparatus to classify, based on the tumor fraction for the sample, the sample as tumor or normal further cause the apparatus to determine, based on the tumor fraction for the sample exceeding a threshold, the sample as tumor.

Aspect 29. The apparatus of any one of aspects 16-28, wherein the processor executable instructions that, when executed by the one or more processors, cause the apparatus to classify, based on the likelihood P(D|θ) of observing the molecules from all regions, the sample as tumor or normal further cause the apparatus to: determine, based on the likelihoods P(D|θ) of observing the molecules from all regions, a posterior mean tumor fraction; and classify, based on the posterior mean tumor fraction for the sample, the sample as tumor or normal.

Aspect 30. The apparatus of aspect 29, wherein the processor executable instructions that, when executed by the one or more processors, cause the apparatus to classify, based on the tumor fraction for the sample, the sample as tumor or normal further cause the apparatus to determine, based on the posterior mean tumor fraction for the sample exceeding a threshold, the sample as tumor.

Aspect 31. A non-transitory computer-readable medium storing processor executable instructions that, when executed by at least one computing device, cause the at least one computing device to:

determine, for a sample comprising a plurality of molecules, sample methylation data comprising an indication of a methylation bin of a plurality of methylation bins, a number of CG sites per molecule of the plurality of molecules, and a region identifier; determine, for each molecule of the plurality of molecules, based on: MBD binding calibration data, the indication of the methylation bin, and the number of CG sites, a likelihood P(f|θ,u, v) of observing the molecule in the indicated methylation bin; group, based on the region identifiers, the molecules of the plurality of molecules into regions; determine, for each region, based on the likelihoods P(f|θ,u, v) of observing the molecules in the indicated methylation bins, a likelihood P(Dᵢ|θ, u, v) of observing the molecules of the region in the methylation bins of the plurality of methylation bins; determine, for each region, for each possible normal methylation rate and each possible tumor methylation rate, based on the likelihoods P(Dᵢ|θ, u, v) of observing the molecules of the region in the methylation bins of the plurality of methylation bins, a likelihood P(Dᵢ|θ) of observing all molecules from the region with a given tumor fraction; determine, for the sample, based on the likelihoods P(Dᵢ|θ) of observing all molecules from the regions with the given tumor fractions, a likelihood P(D|θ) of observing the molecules from all regions; and classify, based on the likelihoods P(D|θ) of observing the molecules from all regions, the sample as tumor or normal.

Aspect 32. The non-transitory computer-readable medium of aspect 31, wherein the processor executable instructions that, when executed by the one or more processors, cause the apparatus to determine, for the sample comprising the plurality of molecules, the sample methylation data further cause the at least one computing device to: partition each molecule of the plurality of molecules into a hypermethylation bin, a residual methylation bin, or a hypomethylation bin;

tag each molecule with a molecular barcode indicating the bin for the molecule; sequence the molecules to generate sequencing data; and determine, based on the sequencing data, the indication of the methylation bin of the plurality of methylation bins, the number of CG sites per molecule of the plurality of molecules, and the region identifier.

Aspect 33. The non-transitory computer-readable medium of aspect 31 or 32, wherein the processor executable instructions that, when executed by the one or more processors, cause the apparatus to determine, for each molecule of the plurality of molecules, the likelihood P(f|θ,u,v) of observing the molecule in the indicated methylation bin further the at least one computing device to: determine a tumor methylation rate of tumor derived molecules; determine a normal methylation rate of normal derived molecules; and determine, based on the tumor methylation rate and the normal methylation rate, a portion of tumor derived molecules among all molecules from a region.

Aspect 34. The non-transitory computer-readable medium of any one of aspects 31-33, wherein the processor executable instructions that, when executed by the one or more processors, cause the apparatus to determine, for each molecule of the plurality of molecules, the likelihood P(f|θ,u,v) of observing the molecule in the indicated methylation bin further cause the apparatus to determine a sum of a joint probability that 1) the molecule is tumor derived and the molecule is in a given partition and 2) the probability that the molecule is normal derived and the molecule is in a given partition.

Aspect 35. The non-transitory computer-readable medium of any one of aspects 31-34, wherein the processor executable instructions that, when executed by the one or more processors, cause the apparatus to determine, for each region, for each possible normal and tumor methylation rates, the likelihood P(Dᵢ|θ) of observing all molecules from the region with a given tumor fraction further cause the at least one computing device to: define a prior probability distribution of a normal methylation rate and a tumor methylation rate such that the normal methylation rate is more likely to be low than high while the tumor methylation rate is more likely to be high than low; and determine, based on the prior probability distribution, a sum of a product of: a probability of observing a given normal methylation rate, a probability of observing a given tumor methylation rate, and a probability of observing all molecules.

Aspect 36. The non-transitory computer-readable medium of any one of aspects 31-35, wherein the processor executable instructions that, when executed by the one or more processors, cause the apparatus to determine, for the sample, the likelihood P(D|θ) of observing the molecules from all regions further cause the apparatus to determine a product of the probability of observing all molecules from each region.

Aspect 37. The non-transitory computer-readable medium of any one of aspects 31-36, wherein the processor executable instructions that, when executed by the one or more processors, further cause the apparatus to determine the MBD binding calibration data.

Aspect 38. The non-transitory computer-readable medium of aspect 37, wherein the processor executable instructions that, when executed by the one or more processors, cause the apparatus to determine the MBD binding calibration data further cause the at least one computing device to: determine, for each methylated control molecule of a plurality of methylated control molecules, an indication of a methylation bin of the plurality of methylation bins and a number of CG sites per methylated control molecule of the plurality of methylated control molecules; plot based on the indication of the methylation bin and the number of CG sites of each methylated control molecule, a hypermethylation binding curve; determine, for each unmethylated control molecule of a plurality of unmethylated control molecules, an indication of a methylation bin of the plurality of methylation bins and a number of CG sites per unmethylated control molecule of the plurality of unmethylated control molecules; and plot based on the indication of the methylation bin and the number of CG sites of each unmethylated control molecule, a hypomethylation binding curve.

Aspect 39. The non-transitory computer-readable medium of aspect 37, wherein the processor executable instructions that, when executed by the one or more processors, cause the apparatus to determine the MBD binding calibration data further cause the at least one computing device to: determine, based on a first wash, for each control molecule of a plurality of control molecules, a first indication that the control molecule is bound to MBD or a second indication that the control molecule is not bound to MBD; estimate, for the plurality of control molecules, based on the first indications and the second indications, a first binding energy and a second binding energy; determine, based on a given number of methylated CG sites, the first binding energy, and the second binding energy, a likelihood (P(bound)) of a molecule to bind to MBD after the first wash and a likelihood (P(unbound)) of a molecule to not bind to MBD after the first wash; plot based on the given numbers of methylated CG sites and the likelihood (P(unbound)) of a molecule to not bind to MBD after the first wash, a hypomethylation binding curve; determine, based on a second wash, for each control molecule of the plurality of control molecules having an indication that the control molecule is bound to MBD in the first wash, a third indication that the control molecule is bound to MBD or a fourth indication that the control molecule is not bound to MBD; estimate, for the plurality of control molecules having an indication that the control molecule is bound to MBD in the first wash, based on the third indications and the fourth indications, a third binding energy and a fourth binding energy; determine, based on a given number of methylated CG sites, the third binding energy, and the fourth binding energy, a likelihood (P(hyper)) of a molecule to bind to MBD after the second wash and a likelihood (P(residual)) of a molecule to not bind to MBD after the second wash; and plot based on the given numbers of methylated CG sites and the likelihood (P(hyper)) of a molecule to bind to MBD after the second wash, a hypermethylation binding curve.

Aspect 40. The non-transitory computer-readable medium of aspect 37, wherein the processor executable instructions that, when executed by the one or more processors, cause the apparatus to determine the MBD binding calibration data further cause the at least one computing device to: determine a likelihood of a molecule to bind to MBD after a first wash; determine a likelihood of a molecule to not bind to MBD after the first wash; determine, based on the likelihood of the molecule to bind to MBD after the first wash, a likelihood that the molecule that bound to MBD after the first wash binds to MBD after a second wash; determine, based on the likelihood of the molecule to bind to MBD after the first wash, a likelihood that the molecule that bound to MBD after the first wash does not bind to MBD after the second wash; plot, based on a given number of methylated CG sites and the likelihood of a molecule to not bind to MBD after the first wash, a hypomethylation binding curve; and plot, based on the given number of methylated CG sites and the likelihood that the molecule that bound to MBD after the first wash binds to MBD after the second wash, a hypermethylation binding curve.

Aspect 41. The non-transitory computer-readable medium of aspect 37, wherein the processor executable instructions that, when executed by the one or more processors, cause the apparatus to determine the MBD binding calibration data further cause the at least one computing device to: aggregate, for a plurality of samples, unmethylated CG site count data for a plurality of molecules and associated likelihoods of observing a molecule in a methylation bin of the plurality of methylation bins; and determine, based on the unmethylated CG site count data and the associated likelihoods of observing a molecule in a methylation bin of the plurality of methylation bins, a negative MBD binding curve associated with each of a hypermethylation bin, a residual methylation bin, and a hypomethylation bin.

Aspect 42. The non-transitory computer-readable medium of aspect 41, wherein the processor executable instructions that, when executed by the one or more processors, cause the apparatus to classify, based on the likelihood P(D|θ) of observing the molecules from all regions, the sample as tumor or normal further cause the at least one computing device to: determine, based on the largest likelihood P(D|θ) of observing all molecules from the region with the given tumor fraction, a tumor fraction for the sample; and classify, based on the tumor fraction for the sample, the sample as tumor or normal.

Aspect 43. The non-transitory computer-readable medium of aspect 42, wherein the processor executable instructions that, when executed by the one or more processors, cause the apparatus to classify, based on the tumor fraction for the sample, the sample as tumor or normal further cause the apparatus to determine, based on the tumor fraction for the sample exceeding a threshold, the sample as tumor.

Aspect 44. The non-transitory computer-readable medium of aspect 41, wherein the processor executable instructions that, when executed by the one or more processors, cause the apparatus to classify, based on the likelihood P(D|θ) of observing the molecules from all regions, the sample as tumor or normal further cause the at least one computing device to: determine, based on the likelihoods P(D|θ) of observing the molecules from all regions, a posterior mean tumor fraction; and classify, based on the posterior mean tumor fraction for the sample, the sample as tumor or normal.

Aspect 45. The non-transitory computer-readable medium of aspect 44, wherein the processor executable instructions that, when executed by the one or more processors, cause the apparatus to classify, based on the tumor fraction for the sample, the sample as tumor or normal further cause the apparatus to determine, based on the posterior mean tumor fraction for the sample exceeding a threshold, the sample as tumor.

Aspect 46. A system, comprising: an assay, configured to partition a plurality of molecules for a sample comprising the plurality of molecules; a sequencer, configured to sequence the partitioned plurality of molecules; and at least one computing device, configured to: determine, based on the sequenced partitioned plurality of molecules, sample methylation data comprising an indication of a methylation bin of a plurality of methylation bins, a number of CG sites per molecule of the plurality of molecules, and a region identifier; determine, for each molecule of the plurality of molecules, based on: MBD binding calibration data, the indication of the methylation bin, and the number of CG sites, a likelihood P(f|θ,u, v) of observing the molecule in the indicated methylation bin; group, based on the region identifiers, the molecules of the plurality of molecules into regions; determine, for each region, based on the likelihoods P(f|θ,u, v) of observing the molecules in the indicated methylation bins, a likelihood P(Dᵢ|θ, u,v) of observing the molecules of the region in the methylation bins of the plurality of methylation bins; determine, for each region, for each possible normal methylation rate and each possible tumor methylation rate, based on the likelihoods P(Dᵢ|θ, u, v) of observing the molecules of the region in the methylation bins of the plurality of methylation bins, a likelihood P(Dᵢ|θ) of observing all molecules from the region with a given tumor fraction; determine, for the sample, based on the likelihoods P(Dᵢ|θ) of observing all molecules from the regions with the given tumor fractions, a likelihood P(D|θ) of observing the molecules from all regions; and classify, based on the likelihoods P(D|θ) of observing the molecules from all regions, the sample as tumor or normal.

Aspect 47. The system of aspect 46, wherein the processor executable instructions that, when executed by the one or more processors, cause the apparatus to determine, for the sample comprising the plurality of molecules, the sample methylation data further cause the at least one computing device to: partition each molecule of the plurality of molecules into a hypermethylation bin, a residual methylation bin, or a hypomethylation bin; tag each molecule with a molecular barcode indicating the bin for the molecule; sequence the molecules to generate sequencing data; and determine, based on the sequencing data, the indication of the methylation bin of the plurality of methylation bins, the number of CG sites per molecule of the plurality of molecules, and the region identifier.

Aspect 48. The system of aspect 46 or 47, wherein the processor executable instructions that, when executed by the one or more processors, cause the apparatus to determine, for each molecule of the plurality of molecules, the likelihood P(f|θ, u, v) of observing the molecule in the indicated methylation bin further the at least one computing device to: determine a tumor methylation rate of tumor derived molecules; determine a normal methylation rate of normal derived molecules; and determine, based on the tumor methylation rate and the normal methylation rate, a portion of tumor derived molecules among all molecules from a region.

Aspect 49. The system of any one of aspects 46-48, wherein the processor executable instructions that, when executed by the one or more processors, cause the apparatus to determine, for each molecule of the plurality of molecules, the likelihood P (f|θ,u, v) of observing the molecule in the indicated methylation bin further cause the apparatus to determine a sum of a joint probability that 1) the molecule is tumor derived and the molecule is in a given partition and 2) the probability that the molecule is normal derived and the molecule is in a given partition.

Aspect 50. The system of any one of aspects 46-49, wherein the processor executable instructions that, when executed by the one or more processors, cause the apparatus to determine, for each region, for each possible normal and tumor methylation rates, the likelihood P(Dᵢ|θ) of observing all molecules from the region with a given tumor fraction further cause the at least one computing device to: define a prior probability distribution of a normal methylation rate and a tumor methylation rate such that the normal methylation rate is more likely to be low than high while the tumor methylation rate is more likely to be high than low; and determine, based on the prior probability distribution, a sum of a product of: a probability of observing a given normal methylation rate, a probability of observing a given tumor methylation rate, and a probability of observing all molecules.

Aspect 51. The system of any one of aspects 46-50, wherein the processor executable instructions that, when executed by the one or more processors, cause the apparatus to determine, for the sample, the likelihood P(D|θ) of observing the molecules from all regions further cause the apparatus to determine a product of the probability of observing all molecules from each region.

Aspect 52. The system of any one of aspects 46-51, wherein the processor executable instructions that, when executed by the one or more processors, further cause the apparatus to determine the MBD binding calibration data.

Aspect 53. The system of aspect 52, wherein the processor executable instructions that, when executed by the one or more processors, cause the apparatus to determine the MBD binding calibration data further cause the at least one computing device to: determine, for each methylated control molecule of a plurality of methylated control molecules, an indication of a methylation bin of the plurality of methylation bins and a number of CG sites per methylated control molecule of the plurality of methylated control molecules; plot based on the indication of the methylation bin and the number of CG sites of each methylated control molecule, a hypermethylation binding curve; determine, for each unmethylated control molecule of a plurality of unmethylated control molecules, an indication of a methylation bin of the plurality of methylation bins and a number of CG sites per unmethylated control molecule of the plurality of unmethylated control molecules; and plot based on the indication of the methylation bin and the number of CG sites of each unmethylated control molecule, a hypomethylation binding curve.

Aspect 54. The system of aspect 52, wherein the processor executable instructions that, when executed by the one or more processors, cause the apparatus to determine the MBD binding calibration data further cause the at least one computing device to: determine, based on a first wash, for each control molecule of a plurality of control molecules, a first indication that the control molecule is bound to MBD or a second indication that the control molecule is not bound to MBD; estimate, for the plurality of control molecules, based on the first indications and the second indications, a first binding energy and a second binding energy; determine, based on a given number of methylated CG sites, the first binding energy, and the second binding energy, a likelihood (P(bound)) of a molecule to bind to MBD after the first wash and a likelihood (P(unbound)) of a molecule to not bind to MBD after the first wash; plot based on the given numbers of methylated CG sites and the likelihood (P(unbound)) of a molecule to not bind to MBD after the first wash, a hypomethylation binding curve; determine, based on a second wash, for each control molecule of the plurality of control molecules having an indication that the control molecule is bound to MBD in the first wash, a third indication that the control molecule is bound to MBD or a fourth indication that the control molecule is not bound to MBD; estimate, for the plurality of control molecules having an indication that the control molecule is bound to MBD in the first wash, based on the third indications and the fourth indications, a third binding energy and a fourth binding energy; determine, based on a given number of methylated CG sites, the third binding energy, and the fourth binding energy, a likelihood (P(hyper)) of a molecule to bind to MBD after the second wash and a likelihood (P(residual)) of a molecule to not bind to MBD after the second wash; and plot based on the given numbers of methylated CG sites and the likelihood (P(hyper)) of a molecule to bind to MBD after the second wash, a hypermethylation binding curve.

Aspect 55. The system of aspect 52, wherein the processor executable instructions that, when executed by the one or more processors, cause the apparatus to determine the MBD binding calibration data further cause the at least one computing device to: determine a likelihood of a molecule to bind to MBD after a first wash; determine a likelihood of a molecule to not bind to MBD after the first wash; determine, based on the likelihood of the molecule to bind to MBD after the first wash, a likelihood that the molecule that bound to MBD after the first wash binds to MBD after a second wash; determine, based on the likelihood of the molecule to bind to MBD after the first wash, a likelihood that the molecule that bound to MBD after the first wash does not bind to MBD after the second wash; plot, based on a given number of methylated CG sites and the likelihood of a molecule to not bind to MBD after the first wash, a hypomethylation binding curve; and plot, based on the given number of methylated CG sites and the likelihood that the molecule that bound to MBD after the first wash binds to MBD after the second wash, a hypermethylation binding curve.

Aspect 56. The system of aspect 52, wherein the processor executable instructions that, when executed by the one or more processors, cause the apparatus to determine the MBD binding calibration data further cause the at least one computing device to: aggregate, for a plurality of samples, unmethylated CG site count data for a plurality of molecules and associated likelihoods of observing a molecule in a methylation bin of the plurality of methylation bins; and determine, based on the unmethylated CG site count data and the associated likelihoods of observing a molecule in a methylation bin of the plurality of methylation bins, a negative MBD binding curve associated with each of a hypermethylation bin, a residual methylation bin, and a hypomethylation bin.

Aspect 57. The system of aspect 56, wherein the processor executable instructions that, when executed by the one or more processors, cause the apparatus to classify, based on the likelihood P(D|θ) of observing the molecules from all regions, the sample as tumor or normal further cause the at least one computing device to: determine, based on the largest likelihood P(D|θ) of observing all molecules from the region with the given tumor fraction, a tumor fraction for the sample; and classify, based on the tumor fraction for the sample, the sample as tumor or normal.

Aspect 58. The system of aspect 57, wherein the processor executable instructions that, when executed by the one or more processors, cause the apparatus to classify, based on the tumor fraction for the sample, the sample as tumor or normal further cause the apparatus to determine, based on the tumor fraction for the sample exceeding a threshold, the sample as tumor.

Aspect 59. The system of aspect 56, wherein the processor executable instructions that, when executed by the one or more processors, cause the apparatus to classify, based on the likelihood P(D|θ) of observing the molecules from all regions, the sample as tumor or normal further cause the at least one computing device to: determine, based on the likelihoods P(D|θ) of observing the molecules from all regions, a posterior mean tumor fraction; and classify, based on the posterior mean tumor fraction for the sample, the sample as tumor or normal.

Aspect 60. The system of aspect 59, wherein the processor executable instructions that, when executed by the one or more processors, cause the apparatus to classify, based on the tumor fraction for the sample, the sample as tumor or normal further cause the apparatus to determine, based on the posterior mean tumor fraction for the sample exceeding a threshold, the sample as tumor.

Aspect 61. A method comprising: obtaining, by a computing system including one or more processors and memory, sequencing data including sequencing reads, individual sequencing reads indicating a nucleotide sequence of a nucleic acid included in a sample and indicating a methyl binding domain (MBD) partition of a plurality of MBD partitions, the MBD partition indicating an amount of methylation of cytosine-guanine (CG) regions of the nucleotide sequence, wherein individual CG regions of the nucleic acid sequence include at least a threshold number of cytosine-guanine pairs; analyzing, by the computing system, the sequencing data to determine a first subset of the sequencing reads that include one or more control regions, the one or more control regions having at least a threshold number of methylated CG regions; determining, by the computing system, a first number of sequencing reads of the first subset of sequencing reads that correspond to a first partition of the plurality of partitions, the first partition corresponding to a first range of numbers of methylated CG regions; determining, by the computing system, a second number of sequencing reads of the first subset of sequencing reads that correspond to a second partition of the plurality of partitions, the second partition corresponding to a second range of numbers of methylated CG regions that is different from the first range of numbers of methylated CG regions; generating, by the computing system, MBD binding calibration data that indicates a first number of probabilities of additional nucleic acids being associated with the first partition and a second number of probabilities of the additional nucleic acids included in the sample being associated with the second partition, wherein the MBD binding calibration data is generated based on the first number of sequencing reads of the subset of sequencing reads and the second number of sequencing reads of the subset of sequencing reads; analyzing, by the computing system, the sequencing data to determine a second subset of the sequencing reads that include one or more classification regions, the one or more classification regions including at least one of a first CG region that has at least a first threshold amount of methylation in cells derived from a tumor and a second CG region that has no greater a second threshold amount of methylation in non-tumor derived cells, the first threshold amount of methylation being greater than the second threshold amount of methylation; determining, by the computing system, partition data for the second subset of the sequencing reads based on partition tags included in the second subset of the sequencing reads, the partition data for individual sequencing reads of the second subset of sequencing reads indicating a partition of the plurality of partitions corresponding to the individual sequencing reads and individual partition tags including a nucleotide sequence corresponding to a partition of the plurality of partitions; determining, by the computing system, CG region data that indicates a number of CG regions of individual nucleic acids that correspond to the individual sequencing reads of the second subset of sequencing reads; determining, by the computing system, an amount of methylation of the number of CG regions in individual classification regions of the one or more classification regions based on an analysis of the partition data and the CG region data in relation to the MBD calibration data; and determining, by the computing system, an estimate for tumor fraction of the sample data by maximizing a likelihood of the estimate of the tumor fraction based on the amounts of methylation of the one or more classification regions, the estimate for tumor fraction indicating a number of nucleic acids included in the sample derived from a tumor.

Aspect 62. The method of aspect 61, comprising: analyzing, by the computing system, the sequencing data to determine a third subset of the sequencing reads that include one or more additional control regions, the one or more additional control regions having no greater than an additional threshold number of methylated CG regions, the additional threshold number of methylated CG regions being less than the threshold number of methylated CG regions; determining, by the computing system, a third number of sequencing reads of the third subset of sequencing reads that correspond to the first partition of the plurality of partitions; determining, by the computing system, a fourth number of sequencing reads of the third subset of sequencing reads that correspond to the second partition of the plurality of partitions; determining, by the computing system, a third number of probabilities of the additional nucleic acids being associated with the first partition and a fourth number of probabilities of the additional nucleic acids being associated with the second partition; wherein the MBD binding calibration data includes the third number of probabilities and the fourth number of probabilities.

Aspect 63. The method of any one of aspects 61 or 62, comprising: determining, by the computing system, a respective likelihood for a plurality of candidate amounts of methylation of the number of CG regions in the individual classification regions of the one or more classification regions based on the analysis of the partition data and the CG region data in relation to the MBD calibration data; and wherein the amount of methylation of the number of CG regions in the individual classification regions corresponds to a candidate amount of methylation of the plurality of candidate amounts of methylation of the number of CG regions having a maximum respective likelihood.

Aspect 64. The method of any one of aspects 61-63, comprising: performing, by the computing system, an alignment process to determine amounts of homology between at least a portion of the sequencing reads included in the sequencing data and one or more nucleotide sequences of a reference genome that correspond to the one or more control regions; and determining, by the computing system and based on the alignment process, the first subset of sequencing reads by determining a number of the sequencing reads having at least a threshold amount of homology with at least one control region of the one or more control regions.

Aspect 65. The method of any one of aspects 61-64, comprising: performing, by the computing system, an alignment process to determine amounts of homology between at least a portion of the sequencing reads included in the sequencing data and one or more nucleotide sequences of a reference genome that correspond to the one or more classification regions; and determining, by the computing system and based on the alignment process, the second subset of sequencing reads by determining a number of the sequencing reads having at least a threshold amount of homology with at least one classification region of the one or more classification regions.

Aspect 66. The method of aspect 65, wherein the one or more classification regions correspond to individual probes of an assay that indicate genomic regions related to a presence of a tumor.

Aspect 67. The method of any one of aspects 61-66, comprising: determining, by the computing system, the one or more classification regions by: determining a first plurality of classification regions having at least a first threshold methylation rate in tumor cells; and determining, by the computing system, a second plurality of classification regions having no greater than a second threshold methylation rate in non-tumor cells, the second threshold being less than the first threshold methylation rate.

Aspect 68. The method of any one of aspects 61-67, comprising: determining, by the computing system, a first likelihood of a candidate estimate of the tumor fraction for the sample based on a plurality of first methylation rates of nucleic acids derived from the sample that correspond to a classification region of the one or more classification regions, the plurality of first methylation rates corresponding to methylation rates in tumor cells; and determining, by the computing system, a second likelihood of the candidate estimate of the tumor fraction for the sample based on a plurality of second methylation rates of the nucleic acids derived from the sample that correspond to the classification region of the one or more classification regions, the plurality of second methylation rates corresponding to methylation rates in non-tumor cells and being less than the methylation rates in tumor cells.

Aspect 69. The method of any one of aspects 61-68, comprising: determining, by the computing system, a probability of a presence of a tumor in the subject from which the sample is derived based on the tumor fraction.

Aspect 70. The method of any one of aspects 61-69, wherein the sample includes a first number of nucleic acids derived from blood or tissue of a subject and a second number of synthetic nucleic acid that include nucleotide sequences that correspond to the one or more control regions.

Aspect 71. The method of any one of aspects 61-70, comprising: combining a plurality of nucleic acids derived from at least one of blood or tissue of a subject with a solution including an amount of MBD to produce a nucleic acid-MBD solution; performing a first wash of the nucleic acid-MBD solution with a first solution including a first concentration of sodium chloride (NaCl) to produce a first nucleic acid fraction and a first residual solution, the first nucleic acid fraction including a first portion of the plurality of nucleic acids and the first residual solution including a second portion of the plurality of nucleic acids, the first portion of the plurality of nucleic acids having a first range of binding energies to MBD that are less than a second range of binding energies to MBD of the second portion of the plurality of nucleic acids; performing a second wash of the first residual solution with a second solution including a second concentration of NaCl that is greater than the first concentration of NaCl to produce a second nucleic acid fraction and a second residual solution, the second nucleic acid fraction including a first subset of the second portion of the plurality of nucleic acids and the second residual solution including a second subset of the second portion of the plurality of nucleic acids, the first subset of the second portion of the plurality of nucleic acids having a third range of binding energies to MBD that are less than a fourth range of binding energies to MBD of the second subset of the second portion of the plurality of nucleic acids; and performing a third wash of the second residual solution with a third solution including a third concentration of NaCl that is greater than the second concentration of NaCl to produce a third nucleic acid fraction that includes the second subset of the second portion of the plurality of nucleic acids.

Aspect 72. The method of aspect 71, comprising: determining that the first portion of the plurality of nucleic acids is associated with the first partition of the plurality of partitions; causing a first molecular barcode to attach to the first portion of the plurality of nucleic acids, the first molecular barcode indicating the first partition; determining that the first subset of the second portion of the plurality of nucleic acids is associated with an additional partition of the plurality of partitions; causing a second molecular barcode to attach to the second portion of the plurality of nucleic acids, the second molecular barcode indicating the additional partition; determining that the second subset of the second portion of the plurality of nucleic acids is associated with the second partition; and causing a third molecular barcode to attach to the second subset of the second portion of the plurality of nucleic acids, the third molecular barcode indicating the second partition.

Aspect 73. A computing system comprising: one or more hardware processors; and one or more computer-readable storage media storing computer readable instructions that, when executed by the one or more hardware processors, cause the one or more hardware processors to perform operations comprising: obtaining sequencing data including sequencing reads, individual sequencing reads indicating a nucleic acid sequence of a nucleic acid included in a sample and indicating a methyl binding domain (MBD) partition of a plurality of MBD partitions, the MBD partition indicating an amount of methylation of cytosine-guanine (CG) regions of the nucleic acid sequence, wherein individual CG regions of the nucleic acid sequence include at least a threshold number of cytosine-guanine pairs; analyzing the sequencing data to determine a first subset of the sequencing reads that include one or more control regions, the one or more control regions having at least a threshold number of methylated CG regions; determining a first number of sequencing reads of the first subset of sequencing reads that correspond to a first partition of the plurality of partitions, the first partition corresponding to a first range of numbers of methylated CG regions; determining a second number of sequencing reads of the first subset of sequencing reads that correspond to a second partition of the plurality of partitions, the second partition corresponding to a second range of numbers of methylated CG regions that is different from the first range of numbers of methylated CG regions; generating MBD binding calibration data that indicates a first number of probabilities of additional nucleic acids being associated with the first partition and a second number of probabilities of the additional nucleic acids being associated with the second partition, wherein the MBD binding calibration data is generated based on the first number of sequencing reads of the subset of sequencing reads and the second number of sequencing reads of the subset of sequencing reads; analyzing the sequencing data to determine a second subset of the sequencing reads that include one or more classification regions, the one or more classification regions including at least one of a first CG region that has at least a first threshold amount of methylation in cells derived from a tumor and a second CG region that has no greater a second threshold amount of methylation in non-tumor derived cells, the first threshold amount of methylation being greater than the second threshold amount of methylation; determining partition data for the second subset of the sequencing reads based on partition tags included in the second subset of the sequencing reads, the partition data for individual sequencing reads of the second subset of sequencing reads indicating a partition of the plurality of partitions corresponding to the individual sequencing reads and individual partition tags including a nucleotide sequence corresponding to a partition of the plurality of partitions; determining CG region data that indicates a number of CG regions of individual nucleic acids that correspond to the individual sequencing reads of the second subset of sequencing reads; determining an amount of methylation of the number of CG regions in individual classification regions of the one or more classification regions based on an analysis of the partition data and the CG region data in relation to the MBD calibration data; and determining an estimate for tumor fraction of the sample data by maximizing a likelihood of the estimate of the tumor fraction based on the amounts of methylation of the one or more classification regions, the estimate for tumor fraction indicating a number of nucleic acids included in the sample derived from a tumor.

Aspect 74. The system of aspect 73, wherein the one or more computer-readable storage media store additional computer-readable instructions that, when executed by the one or more hardware processors, perform additional operations comprising: analyzing the sequencing data to determine a third subset of the sequencing reads that include one or more additional control regions, the one or more additional control regions having no greater than an additional threshold number of methylated CG regions, the additional threshold number of methylated CG regions being less than the threshold number of methylated CG regions; determining a third number of sequencing reads of the third subset of sequencing reads that correspond to the first partition of the plurality of partitions; determining a fourth number of sequencing reads of the third subset of sequencing reads that correspond to the second partition of the plurality of partitions; determining a third number of probabilities of the additional nucleic acids being associated with the first partition and a fourth number of probabilities of the additional nucleic acids being associated with the second partition; wherein the MBD binding calibration data includes the third number of probabilities and the fourth number of probabilities.

Aspect 75. The system of aspect 73 or 74, wherein the one or more computer-readable storage media store additional computer-readable instructions that, when executed by the one or more hardware processors, perform additional operations comprising: determining a respective likelihood for a plurality of candidate amounts of methylation of the number of CG regions in the individual classification regions of the one or more classification regions based on the analysis of the partition data and the CG region data in relation to the MBD calibration data; and wherein the amount of methylation of the number of CG regions in the individual classification regions corresponds to a candidate amount of methylation of the plurality of candidate amounts of methylation of the number of CG regions having a maximum respective likelihood.

Aspect 76. The system of any one of aspects 73-75, wherein the one or more computer-readable storage media store additional computer-readable instructions that, when executed by the one or more hardware processors, perform additional operations comprising: performing an alignment process to determine amounts of homology between at least a portion of the sequencing reads included in the sequencing data and one or more nucleotide sequences of a reference genome that correspond to the one or more control regions; and determining, based on the alignment process, the first subset of sequencing reads by determining a number of the sequencing reads having at least a threshold amount of homology with at least one control region of the one or more control regions.

Aspect 77. The system of any one of aspects 73-76, wherein the one or more computer-readable storage media store additional computer-readable instructions that, when executed by the one or more hardware processors, perform additional operations comprising: performing an alignment process to determine amounts of homology between at least a portion of the sequencing reads included in the sequencing data and one or more nucleotide sequences of a reference genome that correspond to the one or more classification regions; and determining, based on the alignment process, the second subset of sequencing reads by determining a number of the sequencing reads having at least a threshold amount of homology with at least one classification region of the one or more classification regions.

Aspect 78. The system of aspect 77, wherein the one or more classification regions correspond to individual probes of an assay that indicate genomic regions related to a presence of a tumor.

Aspect 79. The system of any one of aspects 73-78, wherein the one or more computer-readable storage media store additional computer-readable instructions that, when executed by the one or more hardware processors, perform additional operations comprising: determining the one or more classification regions by: determining a first plurality of classification regions having at least a first threshold methylation rate in tumor cells; and determining a second plurality of classification regions having no greater than a second threshold methylation rate in non-tumor cells, the second threshold being less than the first threshold methylation rate.

Aspect 80. The system of any one of aspects 73-79, wherein the one or more computer-readable storage media store additional computer-readable instructions that, when executed by the one or more hardware processors, perform additional operations comprising: determining a first likelihood of a candidate estimate of the tumor fraction for the sample based on a plurality of first methylation rates of nucleic acids derived from the sample that correspond to a classification region of the one or more classification regions, the plurality of first methylation rates corresponding to methylation rates in tumor cells; and determining a second likelihood of the candidate estimate of the tumor fraction for the sample based on a plurality of second methylation rates of the nucleic acids derived from the sample that correspond to the classification region of the one or more classification regions, the plurality of second methylation rates corresponding to methylation rates in non-tumor cells and being less than the methylation rates in tumor cells.

Aspect 81. The system of any one of aspects 73-80, wherein the one or more computer-readable storage media store additional computer-readable instructions that, when executed by the one or more hardware processors, perform additional operations comprising: determining a probability of a presence of a tumor in the subject from which the sample is derived based on the tumor fraction.

82. One or more non-transitory computer-readable storage media storing computer-executable instructions that, when executed by one or more processors, perform operations comprising: obtaining sequencing data including sequencing reads, individual sequencing reads indicating a nucleic acid sequence of a nucleic acid included in a sample and indicating a methyl binding domain (MBD) partition of a plurality of MBD partitions, the MBD partition indicating an amount of methylation of cytosine-guanine (CG) regions of the nucleic acid sequence, wherein individual CG regions of the nucleic acid sequence include at least a threshold number of cytosine-guanine pairs; analyzing the sequencing data to determine a first subset of the sequencing reads that include one or more control regions, the one or more control regions having at least a threshold number of methylated CG regions; determining a first number of sequencing reads of the first subset of sequencing reads that correspond to a first partition of the plurality of partitions, the first partition corresponding to a first range of numbers of methylated CG regions; determining a second number of sequencing reads of the first subset of sequencing reads that correspond to a second partition of the plurality of partitions, the second partition corresponding to a second range of numbers of methylated CG regions that is different from the first range of numbers of methylated CG regions; generating MBD binding calibration data that indicates a first number of probabilities of additional nucleic acids being associated with the first partition and a second number of probabilities of the additional nucleic acids being associated with the second partition, wherein the MBD binding calibration data is generated based on the first number of sequencing reads of the subset of sequencing reads and the second number of sequencing reads of the subset of sequencing reads; analyzing the sequencing data to determine a second subset of the sequencing reads that include one or more classification regions, the one or more classification regions including at least one of a first CG region that has at least a first threshold amount of methylation in cells derived from a tumor and a second CG region that has no greater a second threshold amount of methylation in non-tumor derived cells, the first threshold amount of methylation being greater than the second threshold amount of methylation; determining partition data for the second subset of the sequencing reads based on partition tags included in the second subset of the sequencing reads, the partition data for individual sequencing reads of the second subset of sequencing reads indicating a partition of the plurality of partitions corresponding to the individual sequencing reads and individual partition tags including a nucleotide sequence corresponding to a partition of the plurality of partitions; determining CG region data that indicates a number of CG regions of individual nucleic acids that correspond to the individual sequencing reads of the second subset of sequencing reads; determining an amount of methylation of the number of CG regions in individual classification regions of the one or more classification regions based on an analysis of the partition data and the CG region data in relation to the MBD calibration data; and determining an estimate for tumor fraction of the sample data by maximizing a likelihood of the estimate of the tumor fraction based on the amounts of methylation of the one or more classification regions, the estimate for tumor fraction indicating a number of nucleic acids included in the sample derived from a tumor.

Aspect 83. The one or more non-transitory computer-readable media of aspect 82, comprising additional computer-readable instructions that, when executed by the one or more processors, perform additional operations comprising: analyzing the sequencing data to determine a third subset of the sequencing reads that include one or more additional control regions, the one or more additional control regions having no greater than an additional threshold number of methylated CG regions, the additional threshold number of methylated CG regions being less than the threshold number of methylated CG regions; determining a third number of sequencing reads of the third subset of sequencing reads that correspond to the first partition of the plurality of partitions; determining a fourth number of sequencing reads of the third subset of sequencing reads that correspond to the second partition of the plurality of partitions; determining a third number of probabilities of the additional nucleic acids being associated with the first partition and a fourth number of probabilities of the additional nucleic acids being associated with the second partition; wherein the MBD binding calibration data includes the third number of probabilities and the fourth number of probabilities.

Aspect 84. The one or more non-transitory computer-readable media of aspect 82 or 83, wherein the one or more computer-readable storage media store additional computer-readable instructions that, when executed by the one or more hardware processors, perform additional operations comprising: determining a respective likelihood for a plurality of candidate amounts of methylation of the number of CG regions in the individual classification regions of the one or more classification regions based on the analysis of the partition data and the CG region data in relation to the MBD calibration data; and wherein the amount of methylation of the number of CG regions in the individual classification regions corresponds to a candidate amount of methylation of the plurality of candidate amounts of methylation of the number of CG regions having a maximum respective likelihood.

Aspect 85. The one or more non-transitory computer-readable media of any one of aspects 82-84, comprising additional computer-readable instructions that, when executed by the one or more hardware processors, perform additional operations comprising: performing an alignment process to determine amounts of homology between at least a portion of the sequencing reads included in the sequencing data and one or more nucleotide sequences of a reference genome that correspond to the one or more control regions; and determining, based on the alignment process, the first subset of sequencing reads by determining a number of the sequencing reads having at least a threshold amount of homology with at least one control region of the one or more control regions.

Aspect 86. The one or more non-transitory computer-readable media of any one of aspects 82-85, comprising additional computer-readable instructions that, when executed by the one or more hardware processors, perform additional operations comprising: performing an alignment process to determine amounts of homology between at least a portion of the sequencing reads included in the sequencing data and one or more nucleotide sequences of a reference genome that correspond to the one or more classification regions; and determining, based on the alignment process, the second subset of sequencing reads by determining a number of the sequencing reads having at least a threshold amount of homology with at least one classification region of the one or more classification regions.

Aspect 87. The one or more non-transitory computer-readable media of aspect 86, wherein the one or more classification regions correspond to individual probes of an assay that indicate genomic regions related to a presence of a tumor.

Aspect 88. The one or more non-transitory computer-readable media of any one of aspects 82-87, comprising additional computer-readable instructions that, when executed by the one or more hardware processors, perform additional operations comprising: determining the one or more classification regions by: determining a first plurality of classification regions having at least a first threshold methylation rate in tumor cells; and determining a second plurality of classification regions having no greater than a second threshold methylation rate in non-tumor cells, the second threshold being less than the first threshold methylation rate.

Aspect 89. The one or more non-transitory computer-readable media of any one of aspects 82-88, comprising additional computer-readable instructions that, when executed by the one or more hardware processors, perform additional operations comprising: determining a first likelihood of a candidate estimate of the tumor fraction for the sample based on a plurality of first methylation rates of nucleic acids derived from the sample that correspond to a classification region of the one or more classification regions, the plurality of first methylation rates corresponding to methylation rates in tumor cells; and determining a second likelihood of the candidate estimate of the tumor fraction for the sample based on a plurality of second methylation rates of the nucleic acids derived from the sample that correspond to the classification region of the one or more classification regions, the plurality of second methylation rates corresponding to methylation rates in non-tumor cells and being less than the methylation rates in tumor cells.

While specific configurations have been described, it is not intended that the scope be limited to the particular configurations set forth, as the configurations herein are intended in all respects to be possible configurations rather than restrictive. Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
obtaining, by a computing system including one or more processors and memory, sequencing data including sequencing reads, individual sequencing reads indicating a nucleotide sequence of a nucleic acid included in a sample and indicating a methyl binding domain (MBD) partition of a plurality of MBD partitions, the MBD partition indicating an amount of methylation of cytosine-guanine (CG) regions of the nucleotide sequence, wherein individual CG regions of the nucleic acid sequence include at least a threshold number of cytosine-guanine pairs;
analyzing, by the computing system, the sequencing data to determine a first subset of the sequencing reads that include one or more control regions, the one or more control regions having at least a threshold number of methylated CG regions;
determining, by the computing system, a first number of sequencing reads of the first subset of sequencing reads that correspond to a first partition of the plurality of partitions, the first partition corresponding to a first range of numbers of methylated CG regions;
determining, by the computing system, a second number of sequencing reads of the first subset of sequencing reads that correspond to a second partition of the plurality of partitions, the second partition corresponding to a second range of numbers of methylated CG regions that is different from the first range of numbers of methylated CG regions;
generating, by the computing system, MBD binding calibration data that indicates a first number of probabilities of additional nucleic acids being associated with the first partition and a second number of probabilities of the additional nucleic acids included in the sample being associated with the second partition, wherein the MBD binding calibration data is generated based on the first number of sequencing reads of the subset of sequencing reads and the second number of sequencing reads of the subset of sequencing reads;
analyzing, by the computing system, the sequencing data to determine a second subset of the sequencing reads that include one or more classification regions, the one or more classification regions including at least one of a first CG region that has at least a first threshold amount of methylation in cells derived from a tumor and a second CG region that has no greater a second threshold amount of methylation in non-tumor derived cells, the first threshold amount of methylation being greater than the second threshold amount of methylation;
determining, by the computing system, partition data for the second subset of the sequencing reads based on partition tags included in the second subset of the sequencing reads, the partition data for individual sequencing reads of the second subset of sequencing reads indicating a partition of the plurality of partitions corresponding to the individual sequencing reads and individual partition tags including a nucleotide sequence corresponding to a partition of the plurality of partitions;
determining, by the computing system, CG region data that indicates a number of CG regions of individual nucleic acids that correspond to the individual sequencing reads of the second subset of sequencing reads;
determining, by the computing system, an amount of methylation of the number of CG regions in individual classification regions of the one or more classification regions based on an analysis of the partition data and the CG region data in relation to the MBD calibration data; and
determining, by the computing system, an estimate for tumor fraction of the sample data by maximizing a likelihood of the estimate of the tumor fraction based on the amounts of methylation of the one or more classification regions, the estimate for tumor fraction indicating a number of nucleic acids included in the sample derived from a tumor.

2. The method of claim 1, comprising:
analyzing, by the computing system, the sequencing data to determine a third subset of the sequencing reads that include one or more additional control regions, the one or more additional control regions having no greater than an additional threshold number of methylated CG regions, the additional threshold number of methylated CG regions being less than the threshold number of methylated CG regions;
determining, by the computing system, a third number of sequencing reads of the third subset of sequencing reads that correspond to the first partition of the plurality of partitions;
determining, by the computing system, a fourth number of sequencing reads of the third subset of sequencing reads that correspond to the second partition of the plurality of partitions;
determining, by the computing system, a third number of probabilities of the additional nucleic acids being associated with the first partition and a fourth number of probabilities of the additional nucleic acids being associated with the second partition;
wherein the MBD binding calibration data includes the third number of probabilities and the fourth number of probabilities.

3. The method of claim 1, comprising:
determining, by the computing system, a respective likelihood for a plurality of candidate amounts of methylation of the number of CG regions in the individual classification regions of the one or more classification regions based on the analysis of the partition data and the CG region data in relation to the MBD calibration data; and
wherein the amount of methylation of the number of CG regions in the individual classification regions corresponds to a candidate amount of methylation of the plurality of candidate amounts of methylation of the number of CG regions having a maximum respective likelihood.

4. The method of claim 1, comprising:
performing, by the computing system, an alignment process to determine amounts of homology between at least a portion of the sequencing reads included in the sequencing data and one or more nucleotide sequences of a reference genome that correspond to the one or more control regions; and
determining, by the computing system and based on the alignment process, the first subset of sequencing reads by determining a number of the sequencing reads having at least a threshold amount of homology with at least one control region of the one or more control regions.

5. The method of claim 1, comprising:
performing, by the computing system, an alignment process to determine amounts of homology between at least a portion of the sequencing reads included in the sequencing data and one or more nucleotide sequences of a reference genome that correspond to the one or more classification regions; and
determining, by the computing system and based on the alignment process, the second subset of sequencing reads by determining a number of the sequencing reads having at least a threshold amount of homology with at least one classification region of the one or more classification regions.

6. The method of claim 5, wherein the one or more classification regions correspond to individual probes of an assay that indicate genomic regions related to a presence of a tumor.

7. The method of claim 1, comprising:
determining, by the computing system, the one or more classification regions by:
determining a first plurality of classification regions having at least a first threshold methylation rate in tumor cells; and
determining, by the computing system, a second plurality of classification regions having no greater than a second threshold methylation rate in non-tumor cells, the second threshold being less than the first threshold methylation rate.

8. The method of claim 1, comprising:
determining, by the computing system, a first likelihood of a candidate estimate of the tumor fraction for the sample based on a plurality of first methylation rates of nucleic acids derived from the sample that correspond to a classification region of the one or more classification regions, the plurality of first methylation rates corresponding to methylation rates in tumor cells; and
determining, by the computing system, a second likelihood of the candidate estimate of the tumor fraction for the sample based on a plurality of second methylation rates of the nucleic acids derived from the sample that correspond to the classification region of the one or more classification regions, the plurality of second methylation rates corresponding to methylation rates in non-tumor cells and being less than the methylation rates in tumor cells.

9. The method of claim 1, comprising:
determining, by the computing system, a probability of a presence of a tumor in the subject from which the sample is derived based on the tumor fraction.

10. The method of claim 1, wherein the sample includes a first number of nucleic acids derived from blood or tissue of a subject and a second number of synthetic nucleic acid that include nucleotide sequences that correspond to the one or more control regions.

11. The method of claim 1, comprising:
combining a plurality of nucleic acids derived from at least one of blood or tissue of a subject with a solution including an amount of MBD to produce a nucleic acid-MBD solution;
performing a first wash of the nucleic acid-MBD solution with a first solution including a first concentration of sodium chloride (NaCl) to produce a first nucleic acid fraction and a first residual solution, the first nucleic acid fraction including a first portion of the plurality of nucleic acids and the first residual solution including a second portion of the plurality of nucleic acids, the first portion of the plurality of nucleic acids having a first range of binding energies to MBD that are less than a second range of binding energies to MBD of the second portion of the plurality of nucleic acids;
performing a second wash of the first residual solution with a second solution including a second concentration of NaCl that is greater than the first concentration of NaCl to produce a second nucleic acid fraction and a second residual solution, the second nucleic acid fraction including a first subset of the second portion of the plurality of nucleic acids and the second residual solution including a second subset of the second portion of the plurality of nucleic acids, the first subset of the second portion of the plurality of nucleic acids having a third range of binding energies to MBD that are less than a fourth range of binding energies to MBD of the second subset of the second portion of the plurality of nucleic acids; and
performing a third wash of the second residual solution with a third solution including a third concentration of NaCl that is greater than the second concentration of NaCl to produce a third nucleic acid fraction that includes the second subset of the second portion of the plurality of nucleic acids.

12. The method of claim 11, comprising:
determining that the first portion of the plurality of nucleic acids is associated with the first partition of the plurality of partitions;
causing a first molecular barcode to attach to the first portion of the plurality of nucleic acids, the first molecular barcode indicating the first partition;
determining that the first subset of the second portion of the plurality of nucleic acids is associated with an additional partition of the plurality of partitions;
causing a second molecular barcode to attach to the second portion of the plurality of nucleic acids, the second molecular barcode indicating the additional partition;
determining that the second subset of the second portion of the plurality of nucleic acids is associated with the second partition; and
causing a third molecular barcode to attach to the second subset of the second portion of the plurality of nucleic acids, the third molecular barcode indicating the second partition.

13. A computing system comprising:
one or more hardware processors; and
one or more computer-readable storage media storing computer readable instructions that, when executed by the one or more hardware processors, cause the one or more hardware processors to perform operations comprising:
obtaining sequencing data including sequencing reads, individual sequencing reads indicating a nucleic acid sequence of a nucleic acid included in a sample and indicating a methyl binding domain (MBD) partition of a plurality of MBD partitions, the MBD partition indicating an amount of methylation of cytosine-guanine (CG) regions of the nucleic acid sequence, wherein individual CG regions of the nucleic acid sequence include at least a threshold number of cytosine-guanine pairs;
analyzing the sequencing data to determine a first subset of the sequencing reads that include one or more control regions, the one or more control regions having at least a threshold number of methylated CG regions;
determining a first number of sequencing reads of the first subset of sequencing reads that correspond to a first partition of the plurality of partitions, the first partition corresponding to a first range of numbers of methylated CG regions;
determining a second number of sequencing reads of the first subset of sequencing reads that correspond to a second partition of the plurality of partitions, the second partition corresponding to a second range of numbers of methylated CG regions that is different from the first range of numbers of methylated CG regions;
generating MBD binding calibration data that indicates a first number of probabilities of additional nucleic acids being associated with the first partition and a second number of probabilities of the additional nucleic acids being associated with the second partition, wherein the MBD binding calibration data is generated based on the first number of sequencing reads of the subset of sequencing reads and the second number of sequencing reads of the subset of sequencing reads;
analyzing the sequencing data to determine a second subset of the sequencing reads that include one or more classification regions, the one or more classification regions including at least one of a first CG region that has at least a first threshold amount of methylation in cells derived from a tumor and a second CG region that has no greater a second threshold amount of methylation in non-tumor derived cells, the first threshold amount of methylation being greater than the second threshold amount of methylation;
determining partition data for the second subset of the sequencing reads based on partition tags included in the second subset of the sequencing reads, the partition data for individual sequencing reads of the second subset of sequencing reads indicating a partition of the plurality of partitions corresponding to the individual sequencing reads and individual partition tags including a nucleotide sequence corresponding to a partition of the plurality of partitions;
determining CG region data that indicates a number of CG regions of individual nucleic acids that correspond to the individual sequencing reads of the second subset of sequencing reads;
determining an amount of methylation of the number of CG regions in individual classification regions of the one or more classification regions based on an analysis of the partition data and the CG region data in relation to the MBD calibration data; and
determining an estimate for tumor fraction of the sample data by maximizing a likelihood of the estimate of the tumor fraction based on the amounts of methylation of the one or more classification regions, the estimate for tumor fraction indicating a number of nucleic acids included in the sample derived from a tumor.

14. The system of claim 13, wherein the one or more computer-readable storage media store additional computer-readable instructions that, when executed by the one or more hardware processors, perform additional operations comprising:
analyzing the sequencing data to determine a third subset of the sequencing reads that include one or more additional control regions, the one or more additional control regions having no greater than an additional threshold number of methylated CG regions, the additional threshold number of methylated CG regions being less than the threshold number of methylated CG regions;
determining a third number of sequencing reads of the third subset of sequencing reads that correspond to the first partition of the plurality of partitions;
determining a fourth number of sequencing reads of the third subset of sequencing reads that correspond to the second partition of the plurality of partitions;
determining a third number of probabilities of the additional nucleic acids being associated with the first partition and a fourth number of probabilities of the additional nucleic acids being associated with the second partition;
wherein the MBD binding calibration data includes the third number of probabilities and the fourth number of probabilities.

15. The system of claim 13, wherein the one or more computer-readable storage media store additional computer-readable instructions that, when executed by the one or more hardware processors, perform additional operations comprising:
determining a respective likelihood for a plurality of candidate amounts of methylation of the number of CG regions in the individual classification regions of the one or more classification regions based on the analysis of the partition data and the CG region data in relation to the MBD calibration data; and
wherein the amount of methylation of the number of CG regions in the individual classification regions corresponds to a candidate amount of methylation of the plurality of candidate amounts of methylation of the number of CG regions having a maximum respective likelihood.

16. The system of claim 13, wherein the one or more computer-readable storage media store additional computer-readable instructions that, when executed by the one or more hardware processors, perform additional operations comprising:
performing an alignment process to determine amounts of homology between at least a portion of the sequencing reads included in the sequencing data and one or more nucleotide sequences of a reference genome that correspond to the one or more control regions; and
determining, based on the alignment process, the first subset of sequencing reads by determining a number of the sequencing reads having at least a threshold amount of homology with at least one control region of the one or more control regions.

17. The system of claim 13, wherein the one or more computer-readable storage media store additional computer-readable instructions that, when executed by the one or more hardware processors, perform additional operations comprising:
performing an alignment process to determine amounts of homology between at least a portion of the sequencing reads included in the sequencing data and one or more nucleotide sequences of a reference genome that correspond to the one or more classification regions; and
determining, based on the alignment process, the second subset of sequencing reads by determining a number of the sequencing reads having at least a threshold amount of homology with at least one classification region of the one or more classification regions.

18. The system of claim 17, wherein the one or more classification regions correspond to individual probes of an assay that indicate genomic regions related to a presence of a tumor.

19. The system of claim 13, wherein the one or more computer-readable storage media store additional computer-readable instructions that, when executed by the one or more hardware processors, perform additional operations comprising:
determining the one or more classification regions by:
determining a first plurality of classification regions having at least a first threshold methylation rate in tumor cells; and
determining a second plurality of classification regions having no greater than a second threshold methylation rate in non-tumor cells, the second threshold being less than the first threshold methylation rate.

20. The system of claim 13, wherein the one or more computer-readable storage media store additional computer-readable instructions that, when executed by the one or more hardware processors, perform additional operations comprising:
determining a first likelihood of a candidate estimate of the tumor fraction for the sample based on a plurality of first methylation rates of nucleic acids derived from the sample that correspond to a classification region of the one or more classification regions, the plurality of first methylation rates corresponding to methylation rates in tumor cells;
determining a second likelihood of the candidate estimate of the tumor fraction for the sample based on a plurality of second methylation rates of the nucleic acids derived from the sample that correspond to the classification region of the one or more classification regions, the plurality of second methylation rates corresponding to methylation rates in non-tumor cells and being less than the methylation rates in tumor cells.

21. The system of claim 13, wherein the one or more computer-readable storage media store additional computer-readable instructions that, when executed by the one or more hardware processors, perform additional operations comprising:
determining a probability of a presence of a tumor in the subject from which the sample is derived based on the tumor fraction.


SEQUENCE LISTING (8 sequences)

SEQ ID NO: 1 - DNA, 26 bp, Artificial Sequence
  Hyper Methylated CG Region Example 1
acgctcgctgtccaatctchcgttcg

SEQ ID NO: 2 - DNA, 21 bp, Artificial Sequence
  Hyper Methylated CG Region Spike In Example
tgcacgttcgctcgcgcgcta

SEQ ID NO: 3 - DNA, 18 bp, Artificial Sequence
  Residual Methylated CG Region Spike In Example
ccagtacacgctatagca

SEQ ID NO: 4 - DNA, 23 bp, Artificial Sequence
  Hypo Methylated CG Region Spike In Example
ctgatcatcaaactatatagcta

SEQ ID NO: 5 - DNA, 26 bp, Artificial Sequence
  Hyper Methylated CG Region Example 2
tgctagctcgttacgctcgcgcgcta

SEQ ID NO: 6 - DNA, 28 bp, Artificial Sequence
  Hyper Methylated CG Region Example 3
catgcagtacgcgctatatatagccgcg

SEQ ID NO: 7 - DNA, 27 bp, Artificial Sequence
  Hypo Methylated CG Region Example
tgctagctaattaacccttaatcgcta

SEQ ID NO: 8 - DNA, 28 bp, Artificial Sequence
  Residual Methylated CG Region Example
catgcagtacgcgctatatatagcgtta